SEQUENCE LISTING (373 sequences)

SEQ ID NO: 1 - protein, 104 aa, Canis lupus
GPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKAR

SEQ ID NO: 2 - protein, 104 aa, Canis lupus
GPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKAR

SEQ ID NO: 3 - protein, 104 aa, Canis lupus
GPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTP

SEQ ID NO: 4 - protein, 104 aa, Canis lupus
GPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKAR

SEQ ID NO: 5 - protein, 106 aa, Canis lupus
KPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 6 - protein, 105 aa, Canis lupus
QPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 7 - protein, 105 aa, Canis lupus
QPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 8 - protein, 106 aa, Canis lupus
QPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 9 - protein, 220 aa, Canis lupus
VPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 10 - protein, 219 aa, Canis lupus
APEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 11 - protein, 219 aa, Canis lupus
GCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 12 - protein, 220 aa, Canis lupus
VPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 13 - protein, 110 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAK

SEQ ID NO: 14 - protein, 110 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDK

SEQ ID NO: 15 - protein, 110 aa, Felis catus
VPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAK

SEQ ID NO: 16 - protein, 109 aa, Felis catus
GQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 17 - protein, 109 aa, Felis catus
GQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 18 - protein, 109 aa, Felis catus
GQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 19 - protein, 219 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 20 - protein, 219 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 21 - protein, 219 aa, Felis catus
VPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 22 - protein, 17 aa, Canis lupus
FNECRCTDTPPCPVPEP

SEQ ID NO: 23 - protein, 22 aa, Canis lupus
PKRENGRVPRPPDCPKCPAPEM

SEQ ID NO: 24 - protein, 20 aa, Canis lupus
AKECECKCNCNNCPCPGCGL

SEQ ID NO: 25 - protein, 17 aa, Canis lupus
PKESTCKCISPCPVPES

SEQ ID NO: 26 - protein, 17 aa, Canis lupus
PKESTCKCIPPCPVPES

SEQ ID NO: 27 - protein, 17 aa, Felis catus
KTDHPPGPKPCDCPKCP

SEQ ID NO: 28 - protein, 17 aa, Felis catus
KTDHPPGPKPCDCPKCP

SEQ ID NO: 29 - protein, 17 aa, Felis catus
KTASTIESKTGEGPKCP

SEQ ID NO: 30 - protein, 4 aa, synthetic construct
GGGS

SEQ ID NO: 31 - protein, 4 aa, synthetic construct
SGGG

SEQ ID NO: 32 - protein, 5 aa, synthetic construct
GGGGS

SEQ ID NO: 33 - protein, 5 aa, synthetic construct
SGGGG

SEQ ID NO: 34 - protein, 6 aa, synthetic construct
GGGGGS

SEQ ID NO: 35 - protein, 6 aa, synthetic construct
SGGGGG

SEQ ID NO: 36 - protein, 7 aa, synthetic construct
GGGGGGS

SEQ ID NO: 37 - protein, 7 aa, synthetic construct
SGGGGGG

SEQ ID NO: 38 - protein, 5 aa, synthetic construct
GPGGQ

SEQ ID NO: 39 - protein, 9 aa, synthetic construct
GDSITSGYW

SEQ ID NO: 40 - protein, 16 aa, synthetic construct
YISYSGITDYNPSLKS

SEQ ID NO: 41 - protein, 12 aa, synthetic construct
ARYGNYGYAMDY

SEQ ID NO: 42 - protein, 15 aa, synthetic construct
RASESVDTYGNSFMH

SEQ ID NO: 43 - protein, 7 aa, synthetic construct
RASNLES

SEQ ID NO: 44 - protein, 9 aa, synthetic construct
QQSYEDPWT

SEQ ID NO: 45 - protein, 16 aa, synthetic construct
YISYSGITYYNPSLKS

SEQ ID NO: 46 - protein, 15 aa, synthetic construct
RASESVDTYGNSFIH

SEQ ID NO: 47 - protein, 10 aa, synthetic construct
GYTFTSYVMH

SEQ ID NO: 48 - protein, 17 aa, synthetic construct
YINPKNDGTFYNGKFKG

SEQ ID NO: 49 - protein, 7 aa, synthetic construct
FNYGIAY

SEQ ID NO: 50 - protein, 11 aa, synthetic construct
RASQEISGYLS

SEQ ID NO: 51 - protein, 7 aa, synthetic construct
AASTLDS

SEQ ID NO: 52 - protein, 9 aa, synthetic construct
VQYASYPWT

SEQ ID NO: 53 - protein, 17 aa, synthetic construct
YINPNNDGTFYNGKFKG

SEQ ID NO: 54 - protein, 7 aa, synthetic construct
FYYGFAY

SEQ ID NO: 55 - protein, 9 aa, synthetic construct
LQYASYPWT

SEQ ID NO: 56 - protein, 13 aa, synthetic construct
TVSGFSLSRYSVH

SEQ ID NO: 57 - protein, 9 aa, synthetic construct
GMIWGGGST

SEQ ID NO: 58 - protein, 9 aa, synthetic construct
TYRGYALDY

SEQ ID NO: 59 - protein, 11 aa, synthetic construct
RASQDISNYLN

SEQ ID NO: 60 - protein, 8 aa, synthetic construct
YYTSRLQS

SEQ ID NO: 61 - protein, 9 aa, synthetic construct
QQANTLPLT

SEQ ID NO: 62 - protein, 13 aa, synthetic construct
AASGFTFSSFGMH

SEQ ID NO: 63 - protein, 11 aa, synthetic construct
AYIRSDSSTIY

SEQ ID NO: 64 - protein, 11 aa, synthetic construct
SGYYGSFSLTY

SEQ ID NO: 65 - protein, 12 aa, synthetic construct
SASSSVSSNYLH

SEQ ID NO: 66 - protein, 8 aa, synthetic construct
YRTSNLPS

SEQ ID NO: 67 - protein, 8 aa, synthetic construct
QQGSGMLT

SEQ ID NO: 68 - protein, 5 aa, synthetic construct
NYYMA

SEQ ID NO: 69 - protein, 17 aa, synthetic construct
SISTGGGNTYYRDSVKG

SEQ ID NO: 70 - protein, 8 aa, synthetic construct
HGTLYFDY

SEQ ID NO: 71 - protein, 13 aa, synthetic construct
ERSSGDIGDSYVS

SEQ ID NO: 72 - protein, 7 aa, synthetic construct
VDDQRPS

SEQ ID NO: 73 - protein, 11 aa, synthetic construct
QSYDSNIDGPV

SEQ ID NO: 74 - protein, 5 aa, synthetic construct
DYYMA

SEQ ID NO: 75 - protein, 17 aa, synthetic construct
TISYDGSSTYYRDSVRG

SEQ ID NO: 76 - protein, 13 aa, synthetic construct
GPLTDWAPNWFAY

SEQ ID NO: 77 - protein, 11 aa, synthetic construct
QTSEDIYSGLA

SEQ ID NO: 78 - protein, 7 aa, synthetic construct
GASRLED

SEQ ID NO: 79 - protein, 9 aa, synthetic construct
QQGLKYPNT

SEQ ID NO: 80 - protein, 5 aa, synthetic construct
SNGVS

SEQ ID NO: 81 - protein, 16 aa, synthetic construct
AISSGGSTYYNSVLKS

SEQ ID NO: 82 - protein, 12 aa, synthetic construct
RLSGYNYVPFAY

SEQ ID NO: 83 - protein, 11 aa, synthetic construct
KASQNIYKHLA

SEQ ID NO: 84 - protein, 7 aa, synthetic construct
NANSLQT

SEQ ID NO: 85 - protein, 8 aa, synthetic construct
QQYYSGDT

SEQ ID NO: 86 - protein, 5 aa, synthetic construct
DYAMT

SEQ ID NO: 87 - protein, 17 aa, synthetic construct
SISGSGGNTYYADSVKG

SEQ ID NO: 88 - protein, 16 aa, synthetic construct
DRLSITIRPRYYGLDV

SEQ ID NO: 89 - protein, 16 aa, synthetic construct
RSSQSLLYSIGYNYLD

SEQ ID NO: 90 - protein, 7 aa, synthetic construct
LGSNRAS

SEQ ID NO: 91 - protein, 9 aa, synthetic construct
MQALQTPYT

SEQ ID NO: 92 - protein, 5 aa, synthetic construct
SYAMS

SEQ ID NO: 93 - protein, 17 aa, synthetic construct
TISDGGSYTHYPDNLMG

SEQ ID NO: 94 - protein, 10 aa, synthetic construct
ESYDGYYVAN

SEQ ID NO: 95 - protein, 11 aa, synthetic construct
RASQSISNNLH

SEQ ID NO: 96 - protein, 7 aa, synthetic construct
YASQSIS

SEQ ID NO: 97 - protein, 9 aa, synthetic construct
QQSNSWPQT

SEQ ID NO: 98 - protein, 5 aa, synthetic construct
DYYMN

SEQ ID NO: 99 - protein, 17 aa, synthetic construct
WIFPGSGATYYNERFMG

SEQ ID NO: 100 - protein, 8 aa, synthetic construct
SDWDVGDF

SEQ ID NO: 101 - protein, 16 aa, synthetic construct
RSSRSLLHTNGITYLS

SEQ ID NO: 102 - protein, 7 aa, synthetic construct
QMSNLAS

SEQ ID NO: 103 - protein, 9 aa, synthetic construct
AQTLGLPRT

SEQ ID NO: 104 - protein, 5 aa, synthetic construct
DYNMD

SEQ ID NO: 105 - protein, 17 aa, synthetic construct
NINPNSESTSYNQKFKG

SEQ ID NO: 106 - protein, 11 aa, synthetic construct
DGNRYDAWFAY

SEQ ID NO: 107 - protein, 10 aa, synthetic construct
SASSSVTYMH

SEQ ID NO: 108 - protein, 7 aa, synthetic construct
STSILAS

SEQ ID NO: 109 - protein, 9 aa, synthetic construct
QQRTSYPLT

SEQ ID NO: 110 - protein, 5 aa, synthetic construct
NYWIH

SEQ ID NO: 111 - protein, 17 aa, synthetic construct
RIDPNSGGTKYNEKFKS

SEQ ID NO: 112 - protein, 9 aa, synthetic construct
FGSTYGFAY

SEQ ID NO: 113 - protein, 17 aa, synthetic construct
KSSQSLLNSRTRKNYLA

SEQ ID NO: 114 - protein, 7 aa, synthetic construct
WASTRES

SEQ ID NO: 115 - protein, 8 aa, synthetic construct
KQSYNLYT

SEQ ID NO: 116 - protein, 10 aa, synthetic construct
GYTFTTYGMS

SEQ ID NO: 117 - protein, 17 aa, synthetic construct
WINIYSGIPTYADDFKG

SEQ ID NO: 118 - protein, 6 aa, synthetic construct
FDGPDY

SEQ ID NO: 119 - protein, 17 aa, synthetic construct
KSSQSLLNSVNQKNYLA

SEQ ID NO: 120 - protein, 7 aa, synthetic construct
FASARVS

SEQ ID NO: 121 - protein, 9 aa, synthetic construct
QQYFSTPLT

SEQ ID NO: 122 - protein, 10 aa, synthetic construct
GFNIKNTYMH

SEQ ID NO: 123 - protein, 17 aa, synthetic construct
RIAPANVDTKYAPKFQG

SEQ ID NO: 124 - protein, 11 aa, synthetic construct
IYYDYDGDIDV

SEQ ID NO: 125 - protein, 11 aa, synthetic construct
HASQNINVWLS

SEQ ID NO: 126 - protein, 7 aa, synthetic construct
KASHLHT

SEQ ID NO: 127 - protein, 9 aa, synthetic construct
QQGQSWPLT

SEQ ID NO: 128 - protein, 10 aa, synthetic construct
GYTFTRYNMH

SEQ ID NO: 129 - protein, 17 aa, synthetic construct
TIYPGYGDTSYNQKFKG

SEQ ID NO: 130 - protein, 13 aa, synthetic construct
EFADDYPIPPFDY

SEQ ID NO: 131 - protein, 16 aa, synthetic construct
RSSQNIVHSNGNTYLE

SEQ ID NO: 132 - protein, 7 aa, synthetic construct
KVSNRFS

SEQ ID NO: 133 - protein, 9 aa, synthetic construct
FQGSHVPYT

SEQ ID NO: 134 - protein, 10 aa, synthetic construct
GFSLTSYGVH

SEQ ID NO: 135 - protein, 7 aa, synthetic construct
FASTRVS

SEQ ID NO: 136 - protein, 17 aa, synthetic construct
RIDPANVNTKYAPKFQG

SEQ ID NO: 137 - protein, 11 aa, synthetic construct
IFYDYDGDIDV

SEQ ID NO: 138 - protein, 7 aa, synthetic construct
KASNLHT

SEQ ID NO: 139 - protein, 9 aa, synthetic construct
QQGQSYPLT

SEQ ID NO: 140 - protein, 17 aa, synthetic construct
WINIYSGMPTYADDFKG

SEQ ID NO: 141 - protein, 7 aa, synthetic construct
FASTRIS

SEQ ID NO: 142 - protein, 5 aa, synthetic construct
NYYMH

SEQ ID NO: 143 - protein, 17 aa, synthetic construct
WINPYTGSAFYAQKFRG

SEQ ID NO: 144 - protein, 12 aa, synthetic construct
EPEKFDSDDSDV

SEQ ID NO: 145 - protein, 14 aa, synthetic construct
TGSSSNIGAGYGVH

SEQ ID NO: 146 - protein, 7 aa, synthetic construct
GDSNRPS

SEQ ID NO: 147 - protein, 11 aa, synthetic construct
QSYDNSLSGYV

SEQ ID NO: 148 - protein, 7 aa, synthetic construct
GFTFSSY

SEQ ID NO: 149 - protein, 6 aa, synthetic construct
NTGGTV

SEQ ID NO: 150 - protein, 11 aa, synthetic construct [sequence omitted]

SEQ ID NO: 151 - protein, 11 aa, synthetic construct
GGDNIGSKSVH

SEQ ID NO: 152 - protein, 7 aa, synthetic construct
YDSRRPT

SEQ ID NO: 153 - protein, 14 aa, synthetic construct
CQVWDNSDRASWVF

SEQ ID NO: 154 - protein, 8 aa, synthetic construct
GGSVTSSH

SEQ ID NO: 155 - skipped

SEQ ID NO: 156 - protein, 20 aa, synthetic construct
CARGGGYSGTWKDYYVMDYW

SEQ ID NO: 157 - protein, 11 aa, synthetic construct
GGDNLGSKSVH

SEQ ID NO: 158 - protein, 7 aa, synthetic construct
FDTSRPT

SEQ ID NO: 159 - protein, 13 aa, synthetic construct
CQVWDSSAKASVF

SEQ ID NO: 160 - protein, 7 aa, synthetic construct
GFTFSNF

SEQ ID NO: 161 - protein, 6 aa, synthetic construct
NSGGFN

SEQ ID NO: 162 - protein, 17 aa, synthetic construct
CARDWDTHLDTNWFYYW

SEQ ID NO: 163 - protein, 11 aa, synthetic construct
RGDNIGSKSVH

SEQ ID NO: 164 - protein, 7 aa, synthetic construct
YDSSRPT

SEQ ID NO: 165 - protein, 13 aa, synthetic construct
CQVWDISAKASVF

SEQ ID NO: 166 - protein, 7 aa, synthetic construct
GFTFSYY

SEQ ID NO: 167 - protein, 6 aa, synthetic construct
NSGGFS

SEQ ID NO: 168 - protein, 17 aa, synthetic construct
CASESRWGDSYSGMTYW

SEQ ID NO: 169 - protein, 7 aa, synthetic construct
YDNSRPT

SEQ ID NO: 170 - protein, 7 aa, synthetic construct
GFTFSNY

SEQ ID NO: 171 - protein, 6 aa, synthetic construct
TSDGIV

SEQ ID NO: 172 - protein, 15 aa, synthetic construct
CASGLFLVVGGGTFW

SEQ ID NO: 173 - protein, 7 aa, synthetic construct
SDSSRPT

SEQ ID NO: 174 - protein, 6 aa, synthetic construct
ASGGTV

SEQ ID NO: 175 - protein, 16 aa, synthetic construct
CTRWKGGTFGYGMDYW

SEQ ID NO: 176 - protein, 7 aa, synthetic construct
YDNNRPA

SEQ ID NO: 177 - protein, 6 aa, synthetic construct
TNDGIV

SEQ ID NO: 178 - protein, 7 aa, synthetic construct
GFTFSSN

SEQ ID NO: 179 - protein, 6 aa, synthetic construct
SGDGIY

SEQ ID NO: 180 - protein, 13 aa, synthetic construct
CATGIYPNAFGYW

SEQ ID NO: 181 - protein, 11 aa, synthetic construct
CQVWDSSANVF

SEQ ID NO: 182 - protein, 6 aa, synthetic construct
SSGGTF

SEQ ID NO: 183 - protein, 15 aa, synthetic construct
CASGLFLVVGGGNYW

SEQ ID NO: 184 - protein, 11 aa, synthetic construct
CQVWDSSASVF

SEQ ID NO: 185 - protein, 6 aa, synthetic construct
NSDGRV

SEQ ID NO: 186 - protein, 16 aa, synthetic construct
CARWRGGTFGYGMDYW

SEQ ID NO: 187 - protein, 6 aa, synthetic construct
NSDGRI

SEQ ID NO: 188 - protein, 7 aa, synthetic construct
GFTFHNY

SEQ ID NO: 189 - protein, 6 aa, synthetic construct
SSDGRF

SEQ ID NO: 190 - protein, 15 aa, synthetic construct
CANGLFLVLGGENYW

SEQ ID NO: 191 - protein, 6 aa, synthetic construct
SGDGSF

SEQ ID NO: 192 - protein, 13 aa, synthetic construct
CASGIYPNAFGYW

SEQ ID NO: 193 - protein, 11 aa, synthetic construct
CQVRDSSANVF

SEQ ID NO: 194 - protein, 5 aa, synthetic construct
DYGML

SEQ ID NO: 195 - protein, 17 aa, synthetic construct
YISSGSSTIYYADRVKG

SEQ ID NO: 196 - protein, 5 aa, synthetic construct
GTFAY

SEQ ID NO: 197 - protein, 16 aa, synthetic construct
RSSQSLIYNNGNTYLH

SEQ ID NO: 198 - protein, 9 aa, synthetic construct
SQSTHVPFT

SEQ ID NO: 199 - protein, 5 aa, synthetic construct
DDYMH

SEQ ID NO: 200 - protein, 17 aa, synthetic construct
WIDPENGHTKYASKFQG

SEQ ID NO: 201 - protein, 15 aa, synthetic construct
LRHYYGSSYVSPHYY

SEQ ID NO: 202 - protein, 11 aa, synthetic construct
KASQNVGPNVA

SEQ ID NO: 203 - protein, 7 aa, synthetic construct
SASYRYS

SEQ ID NO: 204 - protein, 9 aa, synthetic construct
QQYNNYPYT

SEQ ID NO: 205 - protein, 17 aa, synthetic construct
DINPNNGDTSYNQKFKG

SEQ ID NO: 206 - protein, 14 aa, synthetic construct
GGVLRYPYYYVMDY

SEQ ID NO: 207 - protein, 16 aa, synthetic construct
RSNKSLLHRNGNTYLY

SEQ ID NO: 208 - protein, 7 aa, synthetic construct
RMSNLAS

SEQ ID NO: 209 - protein, 9 aa, synthetic construct
MQHLEFPFT

SEQ ID NO: 210 - protein, 13 aa, synthetic construct
KGSGRTFTSYNMH

SEQ ID NO: 211 - protein, 14 aa, synthetic construct
WVRQMPGKGLEWMG

SEQ ID NO: 212 - protein, 17 aa, synthetic construct
AIYPLTGDTSYNQKSKL

SEQ ID NO: 213 - protein, 10 aa, synthetic construct
RASRSVPYIH

SEQ ID NO: 214 - protein, 8 aa, synthetic construct
YATSALAS

SEQ ID NO: 215 - protein, 9 aa, synthetic construct
QQWLSNPPT

SEQ ID NO: 216 - protein, 5 aa, synthetic construct
NNNVN

SEQ ID NO: 217 - protein, 16 aa, synthetic construct
GVWAGGATDYNSALKS

SEQ ID NO: 218 - protein, 14 aa, synthetic construct
DGGYSSSTLYAMDA

SEQ ID NO: 219 - protein, 11 aa, synthetic construct
RASEDIYNALA

SEQ ID NO: 220 - protein, 7 aa, synthetic construct
NTDTLHT

SEQ ID NO: 221 - protein, 9 aa, synthetic construct
QHYFHYPRT

SEQ ID NO: 222 - protein, 7 aa, synthetic construct
LIGYDIN

SEQ ID NO: 223 - protein, 16 aa, synthetic construct
MIWGDGTTDYNSALKS

SEQ ID NO: 224 - protein, 13 aa, synthetic construct
GGYYYGTSYYFDY

SEQ ID NO: 225 - protein, 11 aa, synthetic construct
RASQDISNHLN

SEQ ID NO: 226 - protein, 7 aa, synthetic construct
YISRFHS

SEQ ID NO: 227 - protein, 9 aa, synthetic construct
QQSKTLPYT

SEQ ID NO: 228 - protein, 8 aa, synthetic construct
TNNIGILG

SEQ ID NO: 229 - skipped

SEQ ID NO: 230 - protein, 11 aa, synthetic construct
QSFDTTLGAHV

SEQ ID NO: 231 - protein, 8 aa, synthetic construct
GFTFSSHG

SEQ ID NO: 232 - protein, 8 aa, synthetic construct
INSGGSST

SEQ ID NO: 233 - protein, 19 aa, synthetic construct
AKESVGGVVEQLVGPHFDY

SEQ ID NO: 234 - protein, 8 aa, synthetic construct
TMDIDIFG

SEQ ID NO: 235 - skipped

SEQ ID NO: 236 - protein, 12 aa, synthetic construct
QSGDSTLGALAI

SEQ ID NO: 237 - protein, 8 aa, synthetic construct
GFTFSTYG

SEQ ID NO: 238 - protein, 8 aa, synthetic construct
ISSGGSST

SEQ ID NO: 239 - protein, 17 aa, synthetic construct
AGSRYTYAYGGGYEFHF

SEQ ID NO: 240 - protein, 5 aa, synthetic construct
SSWMN

SEQ ID NO: 241 - protein, 17 aa, synthetic construct
QIYPGDGDTNYNGKFKG

SEQ ID NO: 242 - protein, 10 aa, synthetic construct
ARHYDGSTDY

SEQ ID NO: 243 - protein, 11 aa, synthetic construct
RASENIYSNLA

SEQ ID NO: 244 - protein, 7 aa, synthetic construct
AATNLAD

SEQ ID NO: 245 - protein, 9 aa, synthetic construct
QHFWGTPYT

SEQ ID NO: 246 - protein, 7 aa, synthetic construct
FSSYGMH

SEQ ID NO: 247 - protein, 11 aa, synthetic construct
VISYDGSIKYY

SEQ ID NO: 248 - protein, 14 aa, synthetic construct
TGEYSGYDTDPQYS

SEQ ID NO: 249 - protein, 11 aa, synthetic construct
RASQGIGDDLG

SEQ ID NO: 250 - protein, 7 aa, synthetic construct
GTSTLQS

SEQ ID NO: 251 - protein, 9 aa, synthetic construct
LQDSNYPLT

SEQ ID NO: 252 - protein, 7 aa, synthetic construct
GYIFITY

SEQ ID NO: 253 - protein, 6 aa, synthetic construct
FPASGS

SEQ ID NO: 254 - protein, 12 aa, synthetic construct
GDGNYALDAMDY

SEQ ID NO: 255 - protein, 15 aa, synthetic construct
RASESVDSYGNSFMH

SEQ ID NO: 256 - protein, 7 aa, synthetic construct
LASNLES

SEQ ID NO: 257 - protein, 9 aa, synthetic construct
QQNNEDPLT

SEQ ID NO: 258 - protein, 8 aa, synthetic construct
GFTLTQYG

SEQ ID NO: 259 - protein, 9 aa, synthetic construct
VIWATGATD

SEQ ID NO: 260 - protein, 13 aa, synthetic construct
DGWWYATSWYFDV

SEQ ID NO: 261 - protein, 11 aa, synthetic construct
KASQDINHYLN

SEQ ID NO: 262 - protein, 7 aa, synthetic construct
YTSRLHS

SEQ ID NO: 263 - protein, 9 aa, synthetic construct
QQGDHFPRT

SEQ ID NO: 264 - protein, 17 aa, synthetic construct
YISSGGDYIYYADTVKG

SEQ ID NO: 265 - protein, 9 aa, synthetic construct
DPITGTFAY

SEQ ID NO: 266 - protein, 11 aa, synthetic construct
RASQDINNYLN

SEQ ID NO: 267 - protein, 7 aa, synthetic construct
YTSTLHS

SEQ ID NO: 268 - protein, 9 aa, synthetic construct
QQGNTLPWT

SEQ ID NO: 269 - protein, 17 aa, synthetic construct
YISSGGDYFYYADTVKG

SEQ ID NO: 270 - protein, 11 aa, synthetic construct
RASQDITNYLN

SEQ ID NO: 271 - protein, 9 aa, synthetic construct
QQGHMLPWT

SEQ ID NO: 272 - protein, 5 aa, synthetic construct
NYWMN

SEQ ID NO: 273 - protein, 17 aa, synthetic construct
QIYPGHVNTNYNGNFKD

SEQ ID NO: 274 - protein, 12 aa, synthetic construct
SADNSGFVLFAY

SEQ ID NO: 275 - protein, 15 aa, synthetic construct
RASKSVSTSGYSYLH

SEQ ID NO: 276 - protein, 9 aa, synthetic construct
QHSRELPLT

SEQ ID NO: 277 - protein, 17 aa, synthetic construct
NINYDGSSTYYLDSLKS

SEQ ID NO: 278 - protein, 8 aa, synthetic construct
GLTWDFDV

SEQ ID NO: 279 - protein, 11 aa, synthetic construct
KASQDVDTAVA

SEQ ID NO: 280 - protein, 7 aa, synthetic construct
LASTRHT

SEQ ID NO: 281 - protein, 9 aa, synthetic construct
QQYSRFPLT

SEQ ID NO: 282 - protein, 9 aa, synthetic construct
GDSITSGYW

SEQ ID NO: 283 - protein, 16 aa, synthetic construct
YISYSGITDYNPSLKS

SEQ ID NO: 284 - protein, 12 aa, synthetic construct
ARYGNYGYAMDY

SEQ ID NO: 285 - protein, 15 aa, synthetic construct
RASESVDTYGNSFMH

SEQ ID NO: 286 - protein, 7 aa, synthetic construct
RASNLES

SEQ ID NO: 287 - protein, 9 aa, synthetic construct
QQSYEDPWT

SEQ ID NO: 288 - protein, 16 aa, synthetic construct
YISYSGITYYNPSLKS

SEQ ID NO: 289 - protein, 15 aa, synthetic construct
RASESVDTYGNSFIH

SEQ ID NO: 290 - protein, 10 aa, synthetic construct
GYTFTSYVMH

SEQ ID NO: 291 - protein, 17 aa, synthetic construct
YINPKNDGTFYNGKFKG

SEQ ID NO: 292 - protein, 7 aa, synthetic construct
FNYGIAY

SEQ ID NO: 293 - protein, 11 aa, synthetic construct
RASQEISGYLS

SEQ ID NO: 294 - protein, 7 aa, synthetic construct
AASTLDS

SEQ ID NO: 295 - protein, 9 aa, synthetic construct
VQYASYPWT

SEQ ID NO: 296 - protein, 17 aa, synthetic construct
YINPNNDGTFYNGKFKG

SEQ ID NO: 297 - protein, 7 aa, synthetic construct
FYYGFAY

SEQ ID NO: 298 - protein, 9 aa, synthetic construct
LQYASYPWT

SEQ ID NO: 299 - protein, 5 aa, synthetic construct
SSWMN

SEQ ID NO: 300 - protein, 17 aa, synthetic construct
QIYPGDGDTNYNGKFKG

SEQ ID NO: 301 - protein, 10 aa, synthetic construct
ARHYDGSTDY

SEQ ID NO: 302 - protein, 11 aa, synthetic construct
RASENIYSNLA

SEQ ID NO: 303 - protein, 7 aa, synthetic construct
AATNLAD

SEQ ID NO: 304 - protein, 9 aa, synthetic construct
QHFWGTPYT

SEQ ID NO: 305 - protein, 7 aa, synthetic construct
FSSYGMH

SEQ ID NO: 306 - protein, 11 aa, synthetic construct
VISYDGSIKYY

SEQ ID NO: 307 - protein, 14 aa, synthetic construct
TGEYSGYDTDPQYS

SEQ ID NO: 308 - protein, 11 aa, synthetic construct
RASQGIGDDLG

SEQ ID NO: 309 - protein, 7 aa, synthetic construct
GTSTLQS

SEQ ID NO: 310 - protein, 9 aa, synthetic construct
LQDSNYPLT

SEQ ID NO: 311 - protein, 7 aa, synthetic construct
GYIFITY

SEQ ID NO: 312 - protein, 6 aa, synthetic construct
FPASGS

SEQ ID NO: 313 - protein, 12 aa, synthetic construct
GDGNYALDAMDY

SEQ ID NO: 314 - protein, 15 aa, synthetic construct
RASESVDSYGNSFMH

SEQ ID NO: 315 - protein, 7 aa, synthetic construct
LASNLES

SEQ ID NO: 316 - protein, 9 aa, synthetic construct
QQNNEDPLT

SEQ ID NO: 317 - protein, 8 aa, synthetic construct
GFTLTQYG

SEQ ID NO: 318 - protein, 9 aa, synthetic construct
VIWATGATD

SEQ ID NO: 319 - protein, 13 aa, synthetic construct
DGWWYATSWYFDV

SEQ ID NO: 320 - protein, 11 aa, synthetic construct
KASQDINHYLN

SEQ ID NO: 321 - protein, 7 aa, synthetic construct
YTSRLHS

SEQ ID NO: 322 - protein, 9 aa, synthetic construct
QQGDHFPRT

SEQ ID NO: 323 - protein, 5 aa, synthetic construct
DYGMH

SEQ ID NO: 324 - protein, 17 aa, synthetic construct
YISSGSRAVFFADTVKG

SEQ ID NO: 325 - protein, 10 aa, synthetic construct
DRYDGRGFAY

SEQ ID NO: 326 - protein, 11 aa, synthetic construct
RASQSISNNLH

SEQ ID NO: 327 - protein, 7 aa, synthetic construct
YASQSIS

SEQ ID NO: 328 - protein, 9 aa, synthetic construct
QQSNSWPLT

SEQ ID NO: 329 - protein, 5 aa, synthetic construct
SYAMS

SEQ ID NO: 330 - protein, 17 aa, synthetic construct
YISSGGDYIYYADTVKG

SEQ ID NO: 331 - protein, 9 aa, synthetic construct
DPITGTFAY

SEQ ID NO: 332 - protein, 11 aa, synthetic construct
RASQDINNYLN

SEQ ID NO: 333 - protein, 7 aa, synthetic construct
YTSTLHS

SEQ ID NO: 334 - protein, 9 aa, synthetic construct
QQGNTLPWT

SEQ ID NO: 335 - protein, 17 aa, synthetic construct
YISSGGDYFYYADTVKG

SEQ ID NO: 336 - protein, 11 aa, synthetic construct
RASQDITNYLN

SEQ ID NO: 337 - protein, 9 aa, synthetic construct
QQGHMLPWT

SEQ ID NO: 338 - protein, 5 aa, synthetic construct
DYYMA

SEQ ID NO: 339 - protein, 17 aa, synthetic construct
NINYDGSSTYYLDSLKS

SEQ ID NO: 340 - protein, 8 aa, synthetic construct
GLTWDFDV

SEQ ID NO: 341 - protein, 11 aa, synthetic construct
KASQDVDTAVA

SEQ ID NO: 342 - protein, 7 aa, synthetic construct
LASTRHT

SEQ ID NO: 343 - protein, 9 aa, synthetic construct
QQYSRFPLT

SEQ ID NO: 344 - protein, 5 aa, synthetic construct
NNNVN

SEQ ID NO: 345 - protein, 17 aa, synthetic construct
GVWAGGATDYNSALKGR

SEQ ID NO: 346 - protein, 14 aa, synthetic construct
DGGYSSSTLYAMDA

SEQ ID NO: 347 - protein, 11 aa, synthetic construct
RASEDIYNALA

SEQ ID NO: 348 - protein, 7 aa, synthetic construct
NTDTLHT

SEQ ID NO: 349 - protein, 8 aa, synthetic construct
HYFHYPRT

SEQ ID NO: 350 - protein, 5 aa, synthetic construct
SYTIH

SEQ ID NO: 351 - protein, 17 aa, synthetic construct
NINPTSGYTENNQRFKD

SEQ ID NO: 352 - protein, 13 aa, synthetic construct
WGFKYDGEWSFDV

SEQ ID NO: 353 - protein, 11 aa, synthetic construct
RASQGISIWLS

SEQ ID NO: 354 - protein, 7 aa, synthetic construct
KASNLHI

SEQ ID NO: 355 - protein, 9 aa, synthetic construct
LQSQTYPLT

SEQ ID NO: 356 - protein, 5 aa, synthetic construct
YYDIN

SEQ ID NO: 357 - protein, 17 aa, synthetic construct
WIFPGDGGTKYNETFKG

SEQ ID NO: 358 - protein, 14 aa, synthetic construct
ARGGTSVIRDAMDY

SEQ ID NO: 359 - protein, 15 aa, synthetic construct
RASESVDNYGISFMH

SEQ ID NO: 360 - protein, 9 aa, synthetic construct
QQSNKDPLT

SEQ ID NO: 361 - protein, 334 aa, Canis lupus
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPCREELSKNTVSLWCLIKDFFPPDIDVEWQSNGQQEPESKYRTTPPQLDEDGSYFLYSKLSVDKSRWQRGDTFICYVMHEALHNHYTQESLSHSPG

SEQ ID NO: 362 - protein, 334 aa, Canis lupus
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPG

SEQ ID NO: 363 - protein, 18 aa, synthetic construct
PKRENGRVPRPPDCPKCP

SEQ ID NO: 364 - protein, 19 aa, synthetic construct
VPKRENGRVPRPPDCPKCP

SEQ ID NO: 365 - protein, 353 aa, synthetic construct
QVQLVESGGGLVQAGGSLRLSCVASGSTFSNLVMAWYRQAPGKQREWVARISLAGNTIYADSVKGRFTISRDNAKNTVYLQMNSLKIEDTAVYYCSRGGVDWGQGTQVTVSSGPGGQVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSP

SEQ ID NO: 366 - protein, 348 aa, synthetic construct
QVQLVESGGGLVQAGGSLRLSCVASGSTFSNLVMAWYRQAPGKQREWVARISLAGNTIYADSVKGRFTISRDNAKNTVYLQMNSLKIEDTAVYYCSRGGVDWGQGTQVTVSSVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSP

SEQ ID NO: 367 - protein, 354 aa, synthetic construct
QVQLVESGGGLVQAGGSLRLSCVASGSTFSNLVMAWYRQAPGKQREWVARISLAGNTIYADSVKGRFTISRDNAKNTVYLQMNSLKIEDTAVYYCSRGGVDWGQGTQVTVSSGPGGQVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPCREELSKNTVSLWCLIKDFFPPDIDVEWQSNGQQEPESKYRTTPPQLDEDGSYFLYSKLSVDKSRWQRGDTFICYVMHEALHNHYTQESLSHSPG

SEQ ID NO: 368 - protein, 365 aa, synthetic construct
SVQLVESGGGLAQPGGSLRLSCVASGSTFSDNAAGWYRQAPEKQRELVAAISIHDRTIYSDSVRGRFTISRDNVKNTVSLQMDNLGPEDTAVYYCNVGGTYYTFGAYAPGDYWGQGTLVTVSSGPGGQVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVCVLPPSREELSKNTVSLSCAIKDFFPPDIDVEWQSNGQQEPESKYRTTPPQLDEDGSYFLVSKLSVDKSRWQRGDTFICYVMHEALHNRFTQESLSHSPG

SEQ ID NO: 369 - protein, 456 aa, synthetic construct
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPGGSGGSQVQLVESGGGLVQAGGSLRLSCVASGSTFSNLVMAWYRQAPGKQREWVARISLAGNTIYADSVKGRFTISRDNAKNTVYLQMNSLKIEDTAVYYCSRGGVDWGQGTQVTVSSGPGGQ

SEQ ID NO: 370 - protein, 334 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYEAKTSPREEQFNSTYRVVSVLPILHVDWLKGKEFKCKVNSKSLPSPIERTISKAKGQPHEPQVYVLPPAQEELSRNKVSVWCLIKSFHPPDIAVEWEITGQPEPENNYRTTPPQLDSDGTYFVYSKLSVDRSHWQRGNTYTCSVYHEALHSHHTQKSLTQSPG

SEQ ID NO: 371 - protein, 334 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYEAKTSPREEQFNSTYRVVSVLPILHVDWLKGKEFKCKVNSKSLPSPIERTISKAKGQPHEPQVYVLPPAQEELSRNKVSVSCAIKSFHPPDIAVEWEITGQPEPENNYRTTPPQLDSDGTYFVVSKLSVDRSHWQRGNTYTCSVYHEALHSRFTQKSLTQSPG

SEQ ID NO: 372 - protein, 334 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYEAKTSPREEQFNSTYRVVSVLPILHVDWLKGKEFKCKVNSKSLPSPIERTISKAKGQPHEPQVYVLPPCQEELSRNKVSVWCLIKSFHPPDIAVEWEITGQPEPENNYRTTPPQLDSDGTYFVYSKLSVDRSHWQRGNTYTCSVYHEALHSHHTQKSLTQSPG

SEQ ID NO: 373 - protein, 334 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYEAKTSPREEQFNSTYRVVSVLPILHVDWLKGKEFKCKVNSKSLPSPIERTISKAKGQPHEPQVCVLPPAQEELSRNKVSVSCAIKSFHPPDIAVEWEITGQPEPENNYRTTPPQLDSDGTYFVVSKLSVDRSHWQRGNTYTCSVYHEALHSRFTQKSLTQSPG

(19) United States
(12) Patent Application Publication
(10) Pub. No.: US 2026/0234262 A1
(43) Pub. Date: Aug. 13, 2026

(54) BISPECIFIC BINDING AGENTS FOR USE IN COMPANION ANIMALS

(71) Applicant: Invetx, Inc., Boston, MA (US)

(72) Inventors: William BRONDYK, Brewster, MA (US); Leila SEVIGNY, Hopkinton, MA (US)

(21) Appl. No.: 19/149,029

(22) PCT Filed: Jan. 22, 2024
(86) PCT No.: PCT/US2024/012354

§ 371 Date: Jul. 17, 2025

Related U.S. Application Data
(60) Provisional application No. 63/480,791, filed on Jan. 20, 2023.

(51) Int. Cl. C07K 16/28; C07K 16/24

(52) U.S. Cl. CPC C07K 16/2827; C07K 16/247; C07K 2317/22; C07K 2317/24; C07K 2317/31; C07K 2317/569; C07K 2317/92

(57) ABSTRACT

Provided are bispecific binding agents (e.g., bispecific antibodies or Fc constructs) for use in companion animals (e.g., dogs and cats), pharmaceutical compositions comprising such bispecific binding agents, and methods of use thereof.

### SEQUENCE LISTING

The instant application contains a Sequence Listing which has been submitted electronically in XML format and is hereby incorporated by reference in its entirety. Said XML copy, created on Jan. 9, 2024, is named 51682-008WO2_Sequence_Listing_1_9_24.XML and is 328,720 bytes in size.

### FIELD OF INVENTION

This disclosure relates generally to bispecific binding agents (e.g., bispecific antibodies or Fc constructs) for use in companion animals (e.g., dogs and cats), pharmaceutical compositions comprising such bispecific binding agents, and methods of use thereof.

### BACKGROUND OF THE INVENTION

Bispecific binding agents, such as bispecific antibodies capable of binding two or more antigens, have broad therapeutic applications. Bispecific antibodies may include two binding domains, which may be directed against two different antigens or two different epitopes on the same antigen. A variety of bispecific antibody formats have been developed for use in humans, such as tetravalent bispecific antibodies by fusion of, e.g., an IgG antibody and single chain domains, and other formats of bispecific antibodies in which the antibody core structure (e.g., IgA, IgD, IgE, IgG, or IgM) is no longer retained. For example, dia-, tria-, and tetrabodies, minibodies, and several single chain formats (scFv, Bis-scFv) capable of binding two or more antigens have been developed. Such formats may use linkers either to fuse the antibody core (e.g., IgA, IgD, IgE, IgG or IgM) to a binding protein (e.g., scFv) or to fuse, e.g., two Fab fragments or scFvs. Production of bispecific antibodies can be difficult; for example, due to the presence of mispaired byproducts, sophisticated purification procedures may be required to produce bispecific antibodies.

Although a number of bispecific antibodies have been approved or are in clinical development for treatment of humans, at present, there are no approved bispecific antibodies for companion animals (e.g., dogs and cats). Additionally, there is limited guidance in the art regarding approaches to successfully produce bispecific antibodies based on companion animal antibodies. In particular, it remains challenging to increase the overall yield, homogeneity, and stability of bispecific antibodies.

Accordingly, there is a need in the art for bispecific binding agents that can be used for treatment of companion animals.

### SUMMARY OF INVENTION

Provided herein are, inter alia, bispecific binding agents (e.g., bispecific antibodies or Fc constructs) for use in companion animals (e.g., dogs and cats), nucleic acids encoding such bispecific binding agents, vectors, host cells, methods of production, pharmaceutical compositions comprising such bispecific binding agents, and methods of use thereof.

In a first aspect, the invention features a bispecific antibody comprising:
(a) a first binding domain that binds to a first antigen, wherein the first binding domain is linked to a first companion animal Fc region variant; and
(b) a second binding domain that binds to a second antigen, wherein the second binding domain is linked to a second companion animal Fc region variant;
   wherein at least one of the first binding domain and the second binding domain comprises a single-domain antibody.

In some embodiments, the first binding domain and the second binding domain each specifically binds to an antigen independently selected from the group consisting of NGF, TrKA, ADAMTS, IL-1, IL-2, IL-4, IL-4R, Angiotensin type 1 (AT1) receptor, Angiotensin type 2 (AT2) receptor, IL-5, IL-12, IL-13, IL-31, IL-31R, IL-33, CD3, CD20, CD47, CD52, and complement system complex.

In some embodiments, the first binding domain and/or the second binding domain comprises an antibody, an antibody fragment, or a ligand-binding portion of a receptor. In some embodiments, the antibody fragment is selected from the group consisting of Fab, single chain variable fragment (scFv), Fv, Fab′, Fab′-SH, F(ab′)₂, and diabody.

In some embodiments, the single-domain antibody is linked to the first companion animal Fc region variant or the second companion animal Fc region variant either directly or via a peptide linker. In some embodiments, the single-domain antibody is a VHH domain. In one embodiment, the VHH domain comprises a C-terminal residue and the first companion animal Fc region variant or the second companion animal Fc region variant comprises an N-terminal residue, and the C-terminal residue of the VHH domain is linked either directly or via a peptide linker to the N-terminal residue of the first companion animal Fc region variant or the second companion animal Fc region variant. In another embodiment, the VHH domain comprises an N-terminal residue, and the first companion animal Fc region variant or the second companion animal Fc region variant comprises a C-terminal residue, and the N-terminal residue of the VHH domain is linked either directly or via a peptide linker to the C-terminal residue of the first companion animal Fc region variant or the second companion animal Fc region variant.

In some embodiments, the peptide linker comprises an amino acid sequence selected from the group consisting of:
(a) GPGGQ (SEQ ID NO: 38);
(b) PKRENGRVPRPPDCPKCP (SEQ ID NO: 363);
(c) VPKRENGRVPRPPDCPKCP (SEQ ID NO: 364);
(d) FNECRCTDTPPCPVPEP (SEQ ID NO: 22);
(e) PKRENGRVPRPPDCPKCPAPEM (SEQ ID NO: 23);
(f) AKECECKCNCNNCPCPGCGL (SEQ ID NO: 24);
(g) PKESTCKCISPCPVPES (SEQ ID NO: 25);
(h) PKESTCKCIPPCPVPES (SEQ ID NO: 26);
(i) KTDHPPGPKPCDCPKCP (SEQ ID NO: 27); and
(j) KTASTIESKTGEGPKCP (SEQ ID NO: 29).

In some embodiments, the first companion animal Fc region variant and the second companion animal Fc region variant are canine Fc region variants. In some embodiments, the first canine Fc region variant or the second canine Fc region variant comprises an amino acid sequence that is at least 95% identical to an amino acid sequence selected from the group consisting of SEQ ID NOs: 9 to 12. In some embodiments, the first canine Fc region variant or the second canine Fc region variant comprises an amino acid sequence that is at least 95% identical to SEQ ID NO: 9. In some embodiments, the first canine Fc region variant or the second canine Fc region variant comprises an amino acid sequence that is at least 95% identical to SEQ ID NO: 10. In some embodiments, the first canine Fc region variant or the second canine Fc region variant comprises an amino acid sequence that is at least 95% identical to SEQ ID NO: 11.

In some embodiments, the first canine Fc region variant or the second canine Fc region variant comprises an amino acid sequence that is at least 95% identical to SEQ ID NO: 12.

In some embodiments, the first canine Fc region variant and the second canine Fc region variant comprise complementary dimerization selectivity modules that promote dimerization between the first canine Fc region variant and the second canine Fc region variant.

In some embodiments, the first canine Fc region variant and the second canine Fc region variant each comprises a protuberance or a cavity, wherein if the first canine Fc region comprises a protuberance, the second canine Fc region comprises a cavity, and wherein if the first canine Fc region comprises a cavity, the second canine Fc region comprises a protuberance.

In some embodiments, the first canine Fc region variant and the second canine Fc region variant comprise amino acid substitutions selected from the group consisting of:
(a) S354C and T366W in the first canine Fc region variant; and Y349C, T366S, L368A, and Y407V in the second canine Fc region variant;
(b) T366W in the first canine Fc region variant; and T366S, L368A, and Y407V in the second canine Fc region variant;
(c) R392D and K409D in the first canine Fc region variant; and E356K and D399K in the second canine Fc region variant;
(d) S364H and F405A in the first canine Fc region variant; and Y349T and T394F in the second canine Fc region variant;
(e) F405L in the first canine Fc region variant; and K409R in the second canine Fc region variant;
(f) T366L, R392L, and T394W in the first canine Fc region variant; and L351Y, F405A, and Y407V in the second canine Fc region variant;
(g) K360E and K409W in the first canine Fc region variant; and S347R, D399V, and F405T in the second canine Fc region variant;
(h) Y349C, K360E, and K409W in the first canine Fc region variant; and S347R, S354C, D399V, and F405T in the second canine Fc region variant;
(i) K370E and K409W in the first canine Fc region variant; and E357N, D399V, and F405T in the second canine Fc region variant;
(j) K360D, D399M, and Y407A in the first canine Fc region variant; and Q345R, S347R, T366V, and K409V in the second canine Fc region variant;
(k) Y349S, T366M, K370Y, and K409V in the first canine Fc region variant; and E356G, E357D, S364Q, and Y407A in the second canine Fc region variant;
(l) L351 D and L368E in the first canine Fc region variant; and L351 K and T366K in the second canine Fc region variant;
(m) L368D and K370S in the first canine Fc region variant; and E356Q and S364K in the second canine Fc region variant; or
(n) T366Y in the first canine Fc region variant; and T366S, L368A, and Y407T in the second canine Fc region variant;
   wherein the amino acid positions are based on EU numbering.

In some embodiments, the first canine Fc region variant comprises a first charged region and the second canine Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first canine Fc region variant and the second canine Fc region variant further comprise CH1 domains comprising the following amino acid substitutions:
S183D in the first canine Fc region variant; and S183K in the second canine Fc region variant.

In some embodiments, the first canine Fc region variant and the second canine Fc region variant comprise CH3 domains comprising amino acid substitutions selected from the group consisting of:
(a) K409D in the first canine Fc region variant and D399K in the second canine Fc region variant;
(b) K390D and K409D in the first canine Fc region variant; and E356K and D399K in the second canine Fc region variant;
(c) K390D and K409D in the first canine Fc region variant; and E357K and D399K in the second canine Fc region variant; and
(d) K370D and K409D and in the first canine Fc region variant; and E357K and D399K in the second canine Fc region variant.

In some embodiments, the first canine Fc region variant and the second canine Fc region variant further comprise CL domains comprising the following amino acid substitutions:
S176K in the first canine Fc region variant; and S176D in the second canine Fc region variant.

In some embodiments, the first canine Fc region variant and/or the second canine Fc region variants further comprise at least one of the following amino acid substitutions:
(a) 252Y and, optionally, at least one amino acid substitution selected from the group consisting of 251 D or 251E; 285N or 285D; 286D; 307Q; 308P; 315D; 430A or 430K; 433K; 435Y; and 436H;
(b) 252M and, optionally, at least one amino acid substitution selected from the group consisting of 251 D or 251E; 256D or 256F; 285N or 285D; 286D; 307Q; 308P; 315D; 430A or 430K; 433K; 435Y; and 436H;
(c) 434R;
(d) 426Y and, optionally, at least one amino acid substitution selected from the group consisting of 286F, 286W, 286L, or 286Y; 312P; 434R; and 436H;
(e) 426H and, optionally, at least one amino acid substitution selected from the group consisting of 286F, 286W, 286L, or 286Y; 312P; 434R; and 436H;
(f) 426F and, optionally, at least one amino acid substitution selected from the group consisting of 286F, 286W, 286L, or 286Y; 312P; 434R; and 426H; and
(g) 434R and, optionally, at least one amino acid substitution selected from the group consisting of 286L; 286Y; 312P; and 436H;
   wherein the amino acid positions are based on EU numbering.

In other embodiments, the first companion animal Fc region variant and the second companion animal Fc region variant are feline Fc region variants. In some embodiments, the first feline Fc region variant or the second feline Fc region variant comprises an amino acid sequence that is at least 95% identical to an amino acid sequence selected from the group consisting of SEQ ID NOs: 19 to 21. In some embodiments, the first feline Fc region variant or the second feline Fc region variant comprises an amino acid sequence that is at least 95% identical to SEQ ID NO: 19. In some embodiments, the first feline Fc region variant or the second feline Fc region variant comprises an amino acid sequence that is at least 95% identical to SEQ ID NO: 20. In some embodiments, the first feline Fc region variant or the second feline Fc region variant comprises an amino acid sequence that is at least 95% identical to SEQ ID NO: 21.

In some embodiments, the first feline Fc region variant and the second feline Fc region variant comprise complementary dimerization selectivity modules that promote dimerization between the first feline Fc region variant and the second feline Fc region variant.

In some embodiments, the first feline Fc region variant and the second feline Fc region variant each comprises a protuberance or a cavity, wherein if the first feline Fc region comprises a protuberance, the second feline Fc region comprises a cavity, and wherein if the first feline Fc region comprises a cavity, the second feline Fc region comprises a protuberance.

In some embodiments, the first feline Fc region variant and the second feline Fc region variant comprise amino acid substitutions selected from the group consisting of:
(a) T366W in the first feline Fc region variant; and T366S, L368A, and Y407V in the second feline Fc region variant;
(b) T366W in the first feline Fc region variant; and T366S, L368A, and Y398T in the second feline Fc region variant;
(c) A354C and T366W in the first feline Fc region variant; and Y349C, T366S, L368A, and Y407V in the second feline Fc region variant;
(d) R392D and K409D in the first feline Fc region variant; and E356K and D399K in the second feline Fc region variant;
(e) S364H and F405A in the first feline Fc region variant; and Y349T and T394F in the second feline Fc region variant;
(f) F405L in the first feline Fc region variant; and K409R in the second feline Fc region variant;
(g) T366L, R392L, and T394W in the first feline Fc region variant; and L351Y, F405A, and Y407V in the second feline Fc region variant;
(h) R360E and K409W in the first feline Fc region variant; and Q347R, D399V, and F405T in the second feline Fc region variant;
(i) Y349C, R360E, and K409W in the first feline Fc region variant; and Q347R, A354C, D399V, F405T in the second feline Fc region variant;
(j) K370E and K409W in the first feline Fc region variant; and E357N, D399V, and F405T in the second feline Fc region variant;
(k) R360D, D399M, and Y407A in the first feline Fc region variant; and E345R, Q347R, T366V, and K409V in the second feline Fc region variant;
(l) Y349S, K370Y, T366M, and K409V in the first feline Fc region variant; and E356G, E357D, S364Q, and Y407A in the second feline Fc region variant;
(m) L351 D and L368E in the first feline Fc region variant; and L351K and T366K in the second feline Fc region variant;
(n) L368D and K370S in the first feline Fc region variant; and E356Q and S364K in the second feline Fc region variant; and
(o) T366Y in the first feline Fc region variant; and T366S, L368A, and Y407T in the second feline Fc region variant;
   wherein the amino acid positions are based on EU numbering.

In some embodiments, the first feline Fc region variant comprises a first charged region and the second feline Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first feline Fc region variant and the second feline Fc region variant further comprise CH1 domains comprising the following amino acid substitutions:
S183D in the first feline Fc region variant; and S183K in the second feline Fc region variant.

In some embodiments, the first feline Fc region variant and the second feline Fc region variant comprise CH3 domains comprising amino acid substitutions selected from the group consisting of:
(a) K409D in the first feline Fc region variant and D399K in the second feline Fc region variant; and
(b) K370D and K409D and in the first feline Fc region variant; and E357K and D399K in the second feline Fc region variant.

In some embodiments, the first feline Fc region variant and the second feline Fc region variant further comprise CL domains comprising the following amino acid substitutions:
S176K in the first feline Fc region variant; and S176D in the second feline Fc region variant.

In some embodiments, the first and/or second feline Fc region variants further comprise at least one of the following amino acid substitutions:
(a) at least one amino acid substitution selected from the group consisting of 286E; 311V; and 428Y;
(b) two or more amino acid substitutions selected from the group consisting of 286E; 311V; and 428Y; and
(c) 286E, 311V, and 428Y;
   wherein the amino acid positions are based on EU numbering.

In another aspect, the invention features an Fc construct comprising:
(a) a first polypeptide comprising a first companion animal Fc region variant; and
(b) a second polypeptide comprising a second companion animal Fc region variant, wherein the first companion animal Fc region variant and the second companion animal Fc region variant comprise complementary dimerization selectivity modules that promote dimerization between the first companion animal Fc region variant and the second companion animal Fc region variant, and wherein the first polypeptide or the second polypeptide does not comprise an antibody.

In some embodiments, the Fc construct further comprises a protein selected from the group consisting of EPO, CTLA4, LFA3, VEGFR1, VEGFR3, IL-1R, IL-4R, GLP-1 receptor agonist, and thrombopoietin binding peptide.

In some embodiments, the first companion animal Fc region variant and the second companion animal Fc region variant are canine Fc region variants. In some embodiments, the first canine Fc region variant or the second canine Fc region variant comprises an amino acid sequence that is at least 95% identical to an amino acid sequence selected from the group consisting of SEQ ID NOs: 9 to 12.

In some embodiments, the first canine Fc region variant and the second canine Fc region variant each comprises a protuberance or a cavity, wherein if the first canine Fc region comprises a protuberance, the second canine Fc region comprises a cavity, and wherein if the first canine Fc region comprises a cavity, the second canine Fc region comprises a protuberance.

In some embodiments, the first canine Fc region variant and the second canine Fc region variant each comprises amino acid substitutions selected from the group consisting of:
(a) S354C and T366W in the first canine Fc region variant; and T366S, L368A, Y407V, and Y349C in the second canine Fc region variant;
(b) T366W in the first canine Fc region variant; and T366S, L368A, and Y407V in the second canine Fc region variant;
(c) R392D and K409D in the first canine Fc region variant; and E356K and D399K in the second canine Fc region variant;
(d) S364H and F405A in the first canine Fc region variant; and Y349T and T394F in the second canine Fc region variant;
(e) F405L in the first canine Fc region variant; and K409R in the second canine Fc region variant; (f) T366L, R392L, and T394W in the first canine Fc region variant; and L351Y, F405A, and Y407V in the second canine Fc region variant;
(g) K360E and K409W in the first canine Fc region variant; and S347R, D399V, and F405T in the second canine Fc region variant;
(h) Y349C, K360E, and K409W in the first canine Fc region variant; and S347R, S354C, D399V, and F405T in the second canine Fc region variant;
(i) K370E and K409W in the first canine Fc region variant; and E357N, D399V, and F405T in the second canine Fc region variant;
(j) K360D, D399M, and Y407A in the first canine Fc region variant; and Q345R, S347R, T366V, and K409V in the second canine Fc region variant;
(k) Y349S, T366M, K370Y, and K409V in the first canine Fc region variant; and E356G, E357D, S364Q, and Y407A in the second canine Fc region variant;
(l) L351 D and L368E in the first canine Fc region variant; and L351K and T366K in the second canine Fc region variant;
(m) L368D and K370S in the first canine Fc region variant; and E356Q and S364K in the second canine Fc region variant; or
(n) T366Y in the first canine Fc region variant; and T366S, L368A, and Y407T in the second canine Fc region variant;
   wherein the amino acid positions are based on EU numbering.

In some embodiments, the first canine Fc region variant comprises a first charged region and the second canine Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first canine Fc region variant and the second canine Fc region variant further comprise CH1 domains comprising the following amino acid substitutions:
S183D in the first canine Fc region variant; and S183K in the second canine Fc region variant.

In some embodiments, the first canine Fc region variant and the second canine Fc region variant comprise CH3 domains comprising amino acid substitutions selected from the group consisting of:
(a) K409D in the first canine Fc region variant and D399K in the second canine Fc region variant;
(b) K390D and K409D in the first canine Fc region variant; and E356K and D399K in the second canine Fc region variant;
(c) K390D and K409D in the first canine Fc region variant; and E357K and D399K in the second canine Fc region variant; and
(d) K370D and K409D and in the first canine Fc region variant; and E357K and D399K in the second canine Fc region variant.

In some embodiments, the first canine Fc region variant and the second canine Fc region variant further comprise CL domains comprising the following amino acid substitutions:
S176K in the first canine Fc region variant; and S176D in the second canine Fc region variant.

In some embodiments, the first canine Fc region variant and the second canine Fc region variant further comprise at least one of the following amino acid substitutions:
(a) 252Y and, optionally, at least one amino acid substitution selected from the group consisting of 251 D or 251E; 285N or 285D; 286D; 307Q; 308P; 315D; 430A or 430K; 433K; 435Y; and 436H;
(b) 252M and, optionally, at least one amino acid substitution selected from the group consisting of 251 D or 251E; 256D or 256F; 285N or 285D; 286D; 307Q; 308P; 315D; 430A or 430K; 433K; 435Y; and 436H;
(c) 434R;
(d) 426Y and, optionally, at least one amino acid substitution selected from the group consisting of 286F, 286W, 286L, or 286Y; 312P; 434R; and 436H;
(e) 426H and, optionally, at least one amino acid substitution selected from the group consisting of 286F, 286W, 286L, or 286Y; 312P; 434R; and 436H;
(f) 426F and, optionally, at least one amino acid substitution selected from the group consisting of 286F, 286W, 286L, or 286Y; 312P; 434R; and 426H; and
(g) 434R and, optionally, at least one amino acid substitution selected from the group consisting of 286L; 286Y; 312P; and 436H;
   wherein the amino acid positions are based on EU numbering.

In other embodiments, the first companion animal Fc region variant and the second companion animal Fc region variant are feline Fc region variants. In some embodiments, the first feline Fc region variant or the second feline Fc region variant comprises an amino acid sequence that is at least 95% identical to an amino acid sequence selected from the group consisting of SEQ ID NOs: 19 to 21.

In some embodiments, the first feline Fc region variant and the second feline Fc region variant each comprises a protuberance or a cavity, wherein if the first feline Fc region comprises a protuberance, the second feline Fc region comprises a cavity, and wherein if the first feline Fc region comprises a cavity, the second feline Fc region comprises a protuberance.

In some embodiments, the first feline Fc region variant and the second feline Fc region variant comprise amino acid substitutions selected from the group consisting of:
(a) T366W in the first feline Fc region variant; and T366S, L368A, and Y407V in the second feline Fc region variant;
(b) T366W in the first feline Fc region variant; and T366S, L368A, and Y398T in the second feline Fc region variant;
(c) A354C and T366W in the first feline Fc region variant; and Y349C, T366S, L368A, and Y407V in the second feline Fc region variant;
(d) R392D and K409D in the first feline Fc region variant; and E356K and D399K in the second feline Fc region variant;
(e) S364H and F405A in the first feline Fc region variant; and Y349T and T394F in the second feline Fc region variant;
(f) F405L in the first feline Fc region variant; and K409R in the second feline Fc region variant;
(g) T366L, R392L, and T394W in the first feline Fc region variant; and L351Y, F405A, and Y407V in the second feline Fc region variant;
(h) R360E and K409W in the first feline Fc region variant; and Q347R, D399V, and F405T in the second feline Fc region variant;
(i) Y349C, R360E, and K409W in the first feline Fc region variant; and Q347R, A354C, D399V, F405T in the second feline Fc region variant;
(j) K370E and K409W in the first feline Fc region variant; and E357N, D399V, and F405T in the second feline Fc region variant;
(k) R360D, D399M, and Y407A in the first feline Fc region variant; and E345R, Q347R, T366V, and K409V in the second feline Fc region variant;
(l) Y349S, K370Y, T366M, and K409V in the first feline Fc region variant; and E356G, E357D, S364Q, and Y407A in the second feline Fc region variant;
(m) L351 D and L368E in the first feline Fc region variant; and L351K and T366K in the second feline Fc region variant;
(n) L368D and K370S in the first feline Fc region variant; and E356Q and S364K in the second feline Fc region variant; and
(o) T366Y in the first feline Fc region variant; and T366S, L368A, and Y407T in the second feline Fc region variant;
   wherein the amino acid positions are based on EU numbering.

In some embodiments, the first feline Fc region variant comprises a first charged region and the second feline Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first feline Fc region variant and the second feline Fc region variant further comprise CH1 domains comprising the following amino acid substitutions:
S183D in the first feline Fc region variant; and S183K in the second feline Fc region variant.

In some embodiments, the first feline Fc region variant and the second feline Fc region variant comprise CH3 domains comprising amino acid substitutions selected from the group consisting of:
(a) K409D in the first feline Fc region variant and D399K in the second feline Fc region variant; and
(b) K370D and K409D and in the first feline Fc region variant; and E357K and D399K in the second feline Fc region variant.

In some embodiments, the first feline Fc region variant and the second feline Fc region variant further comprise CL domains comprising the following amino acid substitutions:
S176K in the first feline Fc region variant; and S176D in the second feline Fc region variant.

In some embodiments, the first feline Fc region variant and the second feline Fc region variant further comprise at least one of the following amino acid substitutions:
(a) at least one amino acid substitution selected from the group consisting of 286E; 311V; and 428Y;
(d) two or more amino acid substitutions selected from the group consisting of 286E; 311V; and 428Y; and
(c) 286E, 311V, and 428Y;
   wherein the amino acid positions are based on EU numbering.

In another aspect, the invention features a pharmaceutical composition comprising (i) any one of the bispecific antibodies disclosed herein or any one of the Fc constructs disclosed herein, and (ii) a pharmaceutically acceptable carrier.

In another aspect, the invention features a nucleic acid or nucleic acids encoding any one of the bispecific antibodies disclosed herein or any one of the Fc constructs disclosed herein.

In another aspect, the invention features an expression vector or expression vectors comprising a nucleic acid or nucleic acids encoding any one of the bispecific antibodies disclosed herein or any one of the Fc constructs disclosed herein.

In another aspect, the invention features a host cell comprising a nucleic acid or nucleic acids encoding any one of the bispecific antibodies disclosed herein or any one of the Fc constructs disclosed herein, or an expression vector or expression vectors comprising a nucleic acid or nucleic acids encoding any one of the bispecific antibodies disclosed herein or any one of the Fc constructs disclosed herein.

In another aspect, the invention features a method of making a bispecific antibody or an Fc construct, the method comprising:
(a) providing a nucleic acid or nucleic acids of encoding any one of the bispecific antibodies disclosed herein or any one of the Fc constructs disclosed herein;
(b) expressing any one of the nucleic acid or nucleic acids disclosed herein in a host cell culture, thereby producing the bispecific antibody or the Fc construct; and optionally
(c) collecting the bispecific antibody or the Fc construct produced in (ii) from the host cell culture.

In some embodiments, the host cell culture comprises (i) one population of host cells expressing both the first companion animal Fc region variant and the second companion animal Fc region variant or (ii) two populations of host cells comprising a first population expressing the first companion animal Fc region variant and a second population expressing the second companion animal Fc region variant.

In another aspect, the invention features a method of treating or preventing a companion animal disease or disorder in a companion animal in need thereof, the method comprising administering an effective amount of a composition comprising any one of the bispecific antibodies disclosed herein, any one of the Fc constructs comprising canine Fc region variants disclosed herein, or a pharmaceutical composition comprising same.

In another aspect, the invention features a method of treating or preventing a canine disease or disorder in a dog in need thereof, the method comprising administering an effective amount of a composition comprising any one of the bispecific antibodies comprising canine Fc region variants disclosed herein, any one of the Fc constructs comprising canine Fc region variants disclosed herein, or a pharmaceutical composition comprising same.

In some embodiments, the canine disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, arthritis, anemia, or obesity.

In another aspect, the invention features any one of the bispecific antibodies comprising canine Fc region variants disclosed herein, any one of the Fc constructs comprising canine Fc region variants disclosed herein, or a pharmaceutical composition comprising same for use in treatment or prevention of a canine disease or disorder in a dog in need thereof.

In some embodiments, the canine disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, arthritis, anemia, or obesity.

In another aspect, the invention features a method of treating or preventing a feline disease or disorder in a cat in need thereof, the method comprising administering an effective amount of a composition comprising any one of the bispecific antibodies comprising feline Fc region variants disclosed herein, any one of the Fc constructs comprising feline Fc region variants disclosed herein, or a pharmaceutical composition comprising same.

In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the feline disease or disorder is atopic dermatitis, allergic dermatitis, osteoarthritic pain, arthritis, anemia, or obesity.

In another aspect, the invention features any one of the bispecific antibodies comprising feline Fc region variants disclosed herein, any one of the Fc constructs comprising feline Fc region variants disclosed herein, or a pharmaceutical composition comprising same for use in treatment or prevention of a feline disease or disorder in a cat in need thereof.

In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the feline disease or disorder is atopic dermatitis, allergic dermatitis, osteoarthritic pain, arthritis, anemia, or obesity.

### BRIEF DESCRIPTION OF THE DRAWINGS

FIG. 1 depicts chromatograms from analytical size-exclusion chromatography (aSEC) for canine knob-in-hole bispecific antibodies comprising two Fab-Fc chains (006 and 225) with 006 Knob+225 Hole or 006 Hole+225 Knob mutations at the Fc domains. K=knob; H=hole; AU=absorbance unit.

FIG. 2 depicts deconvoluted subunit mass spectra from liquid chromatography mass spectrometry (LC-MS) analysis for peptide-N-glycosidase F (PNGase F)-treated canine knob-in-hole bispecific antibodies comprising two Fab-Fc chains (006 and 225) with 006 Knob+225 Hole or 006 Hole+225 Knob mutations at the Fc domains under reduced or non-reduced conditions. HC=heavy chain; LC=light chain; GaINAc=N-acetylgalactosamine; Gal=galactose; amu=atomic mass unit.

FIG. 3 is a schematic depiction of canine bispecific antibodies as determined by non-reduced intact mass analysis. Both confirmed species contain knob-in-hole mutations.

FIG. 4 depicts a double reference-subtracted BIACORE™ sensorgram for canine knob-in-hole bispecific antibodies comprising two Fab-Fc chains (006 and 225) with 006 Knob+225 Hole or 006 Hole+225 Knob mutations at the Fc domains binding to each respective antigen.

FIG. 5 depicts aSEC chromatograms for canine VHH-Fc (02F09R3, with or without linker) and VHH-Fc knob-in-hole bispecific antibody comprising two VHH-Fc chains (02F09R3 and 01E03R3) with 02F09R3-Knob+01E03R3-Hole mutations at the Fc domains.

FIG. 6 depicts deconvoluted subunit mass spectra from LC-MS analysis for PNGase F-treated canine VHH-Fc (02F09R3, with or without linker) and VHH-Fc knob-in-hole bispecific antibody comprising two VHH-Fc chains (02F09R3 and 01E03R3) with 02F09R3-Knob+01E03R3-Hole mutations at the Fc domains under reduced or non-reduced conditions. PyroQ=pyroglutamate.

FIG. 7 is a schematic depiction of canine VHH-Fc and the VHH-Fc knob-in-hole bispecific antibodies as determined by non-reduced intact mass analysis.

FIG. 8 depicts aSEC chromatograms for canine monoclonal antibody (mAb)/VHH-Fc knob-in-hole bispecific antibodies comprising a Fab-Fc chain (006) and a VHH-Fc chain (02F09R3 or 01E03R3) with 006-Knob+01E03R3-Hole or 02F09R3-Knob+006-Hole mutations at the Fc domains.

FIG. 9 depicts deconvoluted subunit mass spectra from LC-MS analysis for PNGase F-treated canine mAb/VHH-Fc knob-in-hole bispecific antibodies comprising a Fab-Fc chain (006) and a VHH-Fc chain (02F09R3 or 01E03R3) with 006-Knob+01E03R3-Hole or 02F09R3-Knob+006-Hole mutations at the Fc domains under reduced or non-reduced conditions.

FIG. 10 is a schematic depiction of canine mAb/VHH-Fc knob-in-hole bispecific antibodies as determined by non-reduced intact mass analysis. Both confirmed species contained knob-in-hole mutations.

FIG. 11 depicts a double reference-subtracted BIACORE™ sensorgram for the canine mAb/VHH-Fc knob-in-hole bispecific antibody comprising a Fab-Fc chain (006) and a VHH-Fc chain (02F09R3) with 02F09R3-Knob+006-Hole mutations at the Fc domains binding to each respective antigen.

FIG. 12 depicts an aSEC chromatogram for the full monoclonal canine IgGB antibody with a C-terminal linker followed by VHH (006_GGS_02F09R3).

FIG. 13 depicts deconvoluted subunit mass spectra from LC-MS analysis for PNGase F-treated full monoclonal canine IgGB antibody with a C-terminal linker followed by VHH (006_GGS_02F09R3) under reduced or non-reduced conditions.

FIG. 14 is a schematic depiction of full monoclonal canine IgGB antibody with a C-terminal linker followed by VHH as determined by non-reduced intact mass analysis.

FIG. 15 depicts a double reference-subtracted BIACORE™ sensorgram for full monoclonal canine IgGB antibody with a C-terminal linker followed by VHH (006_GGS_02F09R3) binding to each respective antigen.

FIG. 16 depicts aSEC chromatograms for feline knob-in-hole bispecific antibodies comprising two Fab-Fc chains (076 and 023) with 076 Knob+023 Hole, 076 Hole+023 Knob, 076 Knob A354C+023 Hole Y349C, or 076 Hole Y349C+023 Knob A354C mutations at the Fc domains binding to each respective antigen. C=cysteine mutation (A354C or Y349C).

FIG. 17 depicts deconvoluted subunit mass spectra from LC-MS analysis for PNGase F-treated feline knob-in-hole bispecific antibodies comprising two Fab-Fc chains (076 and 023) with 076 Knob+023 Hole, 076 Hole+023 Knob, 076 Knob A354C+023 Hole Y349C, or 076 Hole Y349C+023 Knob A354C mutations at the Fc domains under non-reduced conditions.

### DETAILED DESCRIPTION OF THE INVENTION

The invention described herein relates to bispecific binding agents (e.g., bispecific antibodies or Fc constructs) for use in companion animals (e.g., dogs and cats), pharmaceutical compositions comprising such bispecific binding agents, and methods of use thereof. These bispecific binding agents can be used for various therapeutic and diagnostic purposes. In some examples, the disclosure features bispecific binding agents with complementary dimerization selectivity modules that promote dimerization between polypeptide chains, thereby increasing yield and stability.

### Definitions

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. All numerical designations, e.g., pH, Ko, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about” and that a numerical designation may include numerical values that are rounded to the nearest significant figure. It also is to be understood, although not always explicitly stated, that the reagents described herein are merely exemplary and that equivalents of such are known in the art.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or publications, the definition provided herein will control.

It is understood that embodiments of the invention described herein include “comprising,” “consisting,” and “consisting essentially of” aspects and embodiments. As used herein, the singular form “a”, “an,” and “the” includes plural references (e.g., at least one, one or more) unless indicated otherwise. The use of the term “or” herein means “and/or” and is not meant to imply that alternatives are mutually exclusive unless specified otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, “percent (%) amino acid sequence identity,” “% identical,” and “homology” with respect to a nucleic acid or polypeptide sequence are defined as the percentage of nucleotides or amino acid residues in a reference sequence that are identical with the nucleotides or amino acid residues in the specific nucleic acid or polypeptide sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for measuring alignment, including any parameters needed to achieve maximal alignment over the full length of sequences being compared. In some embodiments, a variant has at least 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added or deleted at the N- or C-terminus of the polypeptide. In some embodiments, a variant has at least 50% sequence identity, at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 97% sequence identity, at least 98% sequence identity, or at least 99% sequence identity with the sequence of the reference nucleic acid or polypeptide.

The term “companion animal” refers to an animal suitable to be a companion to humans. In some embodiments, a companion animal is a small mammal, such as a canine, feline, horse, rabbit, ferret, guinea pig, rodent, and the like. In some embodiments, a companion animal is a canine (e.g., a dog) or a feline (e.g., a cat). In some embodiments, a companion animal is a farm animal, such as a horse, cow, pig, and the like.

The term “dimerization selectivity module” refers to a sequence of a Fc domain monomer that facilitates the favored pairing between two Fc domain monomers. “Complementary” dimerization selectivity modules are dimerization selectivity modules that promote or favor the selective interaction of two Fc domain monomers with each other. Complementary dimerization selectivity modules can have the same or different sequences. Exemplary complementary dimerization selectivity modules are described herein.

As used herein, the term “hole” or “cavity” refers to the substitution of at least one of the original amino acid residues in the CH3 domain of an Fc domain monomer with a different amino acid residue having a smaller side chain volume than the original amino acid residue, thus creating a three-dimensional hole or cavity in the CH3 domain. The term “original amino acid residue” refers to a naturally occurring amino acid residue encoded by the genetic code of a wild-type CH3 domain.

As used herein, the term “knob” or “protuberance” refers to the substitution of at least one of the original amino acid residues in the CH3 domain of an Fc domain monomer with a different amino acid residue having a larger side chain volume than the original amino acid residue, thus creating a three-dimensional knob or protuberance in the CH3 domain. The term “original amino acid residues” refers to naturally occurring amino acid residues encoded by the genetic code of a wild-type CH3 domain.

As used herein, the term “knob-in-hole (KiH)” describes an Fc domain or Fc region variant including two Fc domain monomers, in which the first Fc domain monomer includes a “hole” or “cavity” in its CH3 domain, while the second Fc domain monomer includes a “knob” or “protuberance” in its CH3 domain. In a KiH pair, the knob in the CH3 domain of the first Fc domain monomer is positioned such that it interacts with the hole of the CH3 domain of the second Fc domain monomer without significantly perturbing the normal association of the dimer at the inter-CH3 domain interface, thereby promoting heterodimerization of the two Fc domain monomers.

As used herein, the term “charged region” refers to amino acid substitutions in an Fc domain monomer within the ring of charged residues at the interface between CH3 domains that promote dimerization of the Fc domain monomer by forming charge pairs. The term “charge pair” refers to the electrostatic pairing of amino acid residues with opposite charge, e.g., the pairing between a basic amino acid residue and an acidic amino acid residue.

The term “amino acid substitution” refers to the replacement of one amino acid in a polypeptide with another amino acid. In some embodiments, an amino acid substitution is a conservative substitution. Amino acid substitutions may be introduced into a polypeptide screened for a desired activity, for example, retained or improved binding to FcRn, retained or improved antigen binding, decreased immunogenicity, improved ADCC or CDC, or enhanced pharmacokinetics.

The term “conservative substitution” as used herein refers to a substitution of one amino acid residue for another amino acid residue that has similar properties such as charge, hydrophobicity, and/or size. For example, amino acids may be grouped according to common side-chain properties:
hydrophobic: Norleucine (Nle), Met, Ala, Val, Leu, lie;
neutral hydrophilic: Cys, Ser, Thr, Asn, Gln;
acidic: Asp, Glu;
basic: His, Lys, Arg;
rigid: Gly, Pro;
aromatic: Trp, Tyr, Phe.

Conservative substitutions will entail exchanging a member of one of these classes with another member of the same class. Non-conservative substitutions will entail exchanging a member of one of these classes with another class. In some embodiments, a conservative amino acid substitution refers to a substitution that results in similar properties or functions as another amino acid substitution. For example, a conservative amino acid substitution of A426Y can be A426F, A426T, or A426W. Additional, nonlimiting examples for conservative amino acid substitutions are shown in Table 1.

TABLE 1
Examples for conservative amino acid substitutions

| Original residue | Exemplary conservative substitutions |
|---|---|
| Ala (A) | Gly; Val; Leu; Ile; Ser |
| Arg (R) | Lys; His; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Nle |
| Leu (L) | Nle; Ile; Val; Met; Ala; Phe |
| Lys (K) | Arg; His; Gln; Asn |
| Met (M) | Leu; Phe; Ile; Tyr |
| Phe (F) | Trp; Leu; Val; Ile; Ala; Tyr; Met |
| Pro (P) | Ala; Gly |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Nle |

The term “affinity” refers to the strength of the sum total of noncovalent interactions between a single binding site of a molecule (e.g., an antibody or a receptor) and its binding partner (e.g., an antigen or a ligand). Unless indicated otherwise, as used herein, “binding affinity” refers to intrinsic binding affinity which reflects a 1:1 interaction between members of a binding pair (e.g., antibody and antigen, receptor and ligand). The affinity of a molecule X for its partner Y can generally be represented by the dissociation constant (K_{D}). Affinity can be measured by common protein-protein interaction tools known in the art, such as, for example, immunoblot, enzyme-linked immunosorbent assay (ELISA), kinetic exclusion assay (KinExA), biolayer interferometry (BLI), or surface plasmon resonance (SPR) devices. Specific illustrative and exemplary embodiments for measuring binding affinity are described in the following.

“Surface plasmon resonance (SPR)” denotes an optical phenomenon that allows for the analysis of real-time biospecific interactions by detection of alterations in protein concentrations within a biosensor matrix, for example, using the BIAcore™ system (BIAcore International AB, a GE Healthcare company, Uppsala, Sweden and Piscataway, N.J.). For further descriptions, see Jonsson et al., 1993, Ann. Biol. Clin. 51: 19-26.

The term “amino acid sequence” refers a sequence of amino acids residues in a peptide or protein. The terms “polypeptide” and “protein” are used interchangeably to refer to a polymer of amino acid residues and are not limited to a minimum length. Such polymers of amino acid residues may contain natural or unnatural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “antibody” herein is used in the broadest sense and refers to various antibody structures, including but not limited to monoclonal antibodies, polyclonal antibodies, multispecific antibodies (e.g., bispecific antibodies), and antibody fragments (e.g., Fab) so long as they exhibit the desired antigen-binding activity.

The term “bispecific antibody” refers to an antibody derivative that has, in the same antibody molecule, variable regions that recognize two different epitopes. A bispecific antibody may be an antibody that recognizes two different antigens, or an antibody that recognizes two different epitopes on a same antigen.

The term “antibody fragment” refers to a molecule other than a full-length antibody that comprises a portion of a full-length antibody that binds the antigen to which the full-length antibody binds. In some embodiments, antibody fragments include but are not limited to Fab; single chain variable fragment (e.g., scFv); Fv; Fab′; Fab′-SH; F(ab′)₂; nanobody; diabody; and multispecific antibodies formed from antibody fragments.

The terms “full-length antibody” and “whole antibody” are used herein interchangeably to refer to an antibody having a structure substantially similar to a native antibody structure or having heavy chains that contain an Fc region as defined herein.

The terms “nanobody,” “VHH,” “VHH domain,” “VHH antibody fragment,” and “single domain antibody” as interchangeably used herein denote the variable domain of the single heavy chain of antibodies of the type of those found in Camelidae, which are typically found in natural form to lack light chains. Suitable nanobodies will be familiar to persons skilled in the art, illustrated examples of which include nanobodies of camels, dromedaries, llamas, and alpacas. However, the single domain antibody may be from non-Camelidae sources as well.

The term “binding domain” refers to a part of a compound or a molecule that specifically binds to a target epitope, antigen, ligand, or receptor. Binding domains include but are not limited to antibodies (e.g., monoclonal, polyclonal, recombinant, and chimeric antibodies), antibody fragments or portions thereof (e.g., Fab, scFv, Fv, Fab′, Fab′-SH, F(ab′)₂, nanobody, and diabody), receptors or fragments thereof (e.g., an extracellular domain of a canine or feline receptor protein), ligands, aptamers, and other molecules having an identified binding partner.

The term “chimeric” antibody refers to an antibody in which a portion of the heavy and/or light chain is derived from a particular source or species, while the remainder of the heavy and/or light chain is derived from a different source or species.

The terms “Fc region,” “Fc domain,” and “Fc polypeptide” refer to a C-terminal region of an immunoglobulin heavy chain that contains at least a portion of the constant region. The term “Fc region variant” refers to a variant of the Fc region of a companion animal having a substitution or substitutions relative to the wild type companion animal Fc region. The term “Fc domain of the wild type canine IgG” refers to the native Fc region of a canine antibody. The term “canine Fc region variant” refers to a variant of the Fc region of a canine antibody having a substitution or substitutions relative to the wild type canine Fc region. In some embodiments, the canine Fc region sequences are from a canine (e.g., dog) IgG (e.g., IgGA, IgGB, IgGC, or IgGD). The term “Fc domain of the wild type feline IgG” refers to the native Fc region of a feline antibody. The term “feline Fc region variant” refers to a variant of the Fc region of a feline antibody having a substitution or substitutions relative to the wild type feline Fc region. In some embodiments, the feline Fc region sequences are from a feline (e.g., cat) IgG (e.g., IgG1a, IgG1b, or IgG2). In some embodiments, the IgG Fc polypeptide comprises the hinge, CH2, and CH3, but does not comprise CH1 or CL. In some embodiments, the IgG Fc polypeptide comprises CH2 and CH3, but does not comprise CH1, the hinge, or CL. In some embodiments, the IgG Fc polypeptide comprises CH1, hinge, CH2, and CH3, with or without CL. In some embodiments, the IgG Fc polypeptide comprises CH1, hinge, CH2, CH3, and CL. For example, CL may be linked to CH1 via a disulfide bridge. In some embodiments, an Fc polypeptide, such as an IgG Fc polypeptide, lacks one or more C-terminal amino acids, such as 1 to 20, 1 to 15, 1 to 10, 1 to 5, or 1 to 2 amino acids, while retaining biological activity. In some embodiments, the biological activity of an Fc polypeptide is the ability to bind FcRn. Unless otherwise specified herein, numbering of amino acid residues in the Fc region or constant region is according to the EU numbering system, also called the EU index, as described in Kabat et al. Sequences of Proteins of Immunological Interest, 5th Ed. Public Health Service, National Institutes of Health, Bethesda, MD, 1991.

The term “Fc domain monomer” refers to a polypeptide chain that includes at least a hinge domain and second and third antibody constant domains (CH2 and CH3) or functional fragments thereof (e.g., fragments that that capable of (i) dimerizing with another Fc domain monomer to form an Fc domain and (ii) binding to an Fc receptor). The Fc domain monomer can be any immunoglobulin antibody isotype, including, e.g., IgG. Additionally, the Fc domain monomer can be an IgG subtype, e.g., IgGA, IgGB, IgGC, or IgGD in dogs; or IgG1a, IgG1b, or IgG2 in cats. In some embodiments, an Fc domain monomer does not include any portion of an immunoglobulin that is capable of antigen binding, e.g., a variable domain or a complementarity determining region (CDR). In some embodiments, an Fc domain monomer includes a portion of an immunoglobulin that can act as an antigen-recognition region, e.g., a variable domain or a CDR. In some embodiments, an Fc domain monomer includes a single-domain antibody, e.g., a VHH domain.

As used herein, the term “Fc construct” refers to associated polypeptide chains that includes Fc domain monomers or Fc region variants as described herein (e.g., an Fc construct comprising Fc domain monomers or Fc region variants). Fc constructs described herein can include Fc domain monomers that have the same or different sequences. In some embodiments, an Fc construct does not include any portion of an immunoglobulin that is capable of antigen binding, e.g., a variable domain or a complementarity determining region (CDR). In some embodiments, an Fc construct includes a portion of an immunoglobulin that can act as an antigen-recognition region, e.g., a variable domain or a CDR. In some embodiments, an Fc construct includes a single-domain antibody, e.g., a VHH domain.

The term “wild type” refers to a non-mutated version of a polypeptide that occurs in nature, or a fragment thereof. A wild type polypeptide may be produced recombinantly. In some embodiments, a wild type canine (e.g., dog) IgG Fc domain comprises the amino acid sequence of any one of SEQ ID NOs: 9-12.

In other embodiments, a wild type feline (e.g., cat) IgG Fc domain comprises the amino acid sequence of any one of SEQ ID NOs: 19-21.

The term “disease” or “disorder” refers to any condition that would benefit from treatment including, but not limited to, chronic and acute disorders or diseases including those pathological conditions which predispose the mammal to the disorder in question.

The term “cancer” refers to or describes the physiological condition in mammals that is typically characterized by unregulated cell growth/proliferation. Examples of cancer include, but are not limited to, myeloma, carcinoma, lymphoma (e.g., Hodgkin's and non-Hodgkin's lymphoma), blastoma, sarcoma (e.g., hemangiosarcoma, osteosarcoma, soft-tissue sarcoma, and histiocytic sarcoma), leukemia, head and neck squamous cell carcinoma, salivary adenocarcinoma, breast cancer, mastocytoma, melanoma, lung cancer (e.g., small-cell lung cancer, non-small cell lung cancer, adenocarcinoma of the lung, and squamous carcinoma of the lung), cancer of the peritoneum, hepatocellular carcinoma, squamous cell carcinoma, meningioma, glioma, gastric cancer, intestinal cancer, colon cancer, colorectal cancer, pancreatic adenocarcinoma, glioblastoma, cervical cancer, endometrial or uterine carcinoma, ovarian cancer, bladder cancer, prostatic carcinoma, kidney or renal cancer, vulval cancer, thyroid cancer, and transitional cell carcinoma.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “effective amount” of a composition, for example, a polypeptide (e.g., a bispecific binding agent such as a bispecific antibody or an Fc construct) of the present disclosure or a composition (e.g., pharmaceutical composition) thereof, refers to at least the minimum amount required to achieve the desired therapeutic or prophylactic result, such as a measurable improvement or prevention of a particular disorder (e.g., any disorder affecting a canine or a feline, e.g., a cell proliferative disorder, e.g., cancer). An effective amount herein may vary according to factors such as the disease state, age, sex, and weight of the animal, and the ability of the polypeptide (e.g., a bispecific binding agent such as a bispecific antibody or an Fc construct) to elicit a desired response in the animal. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The terms “host cell” and “host cell culture” are used interchangeably and refer to cells into which exogenous nucleic acid has been introduced, including the progeny of such cells. Host cells include bacterial (e.g., E. coli cells) and eukaryotic cells. In some embodiments, host cells include yeast cells (e.g., Pichia (see, e.g., Powers et al., 2001, J Immunol Methods. 251: 123-135), Hanseula, or Saccharomyces). In some embodiments, host cells also include “transformants” and “transformed cells,” which include the primary transformed cell lines (e.g., CHO, 293E, COS, 293T, and HeLa) and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “pharmaceutical composition” refers to a preparation which is in such form as to permit the biological activity of an active ingredient contained therein to be effective, and which contains no additional components which are unacceptably toxic to a subject to which the formulation would be administered.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the bispecific binding agents of the invention are used to delay development of a disease or to slow the progression of a disease.

As used herein, the term “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

The term “epitope” refers to the particular site or sites on an antigen molecule to which an antibody or other binding agent binds. For example, an epitope may be a linear epitope or a conformational epitope.

As used herein, the terms “reduce” and “inhibit” refer to the ability to cause an overall decrease, for example, of 20% or greater, of 50% or greater, or of 75%, 85%, 90%, 95%, or greater, e.g., as compared to a reference or control.

The terms “increase” and “enhance” refer to the ability to cause an overall increase, for example, of 20% or greater, of 50% or greater, or of 75%, 85%, 90%, 95%, or greater, e.g., as compared to a reference or control.

The terms “variable region” and “variable domain” refer to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., 2007, Kuby Immunology, 6th ed. W.H. Freeman and Co., page 91. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., 1993, J. Immunol. 150: 880-887; and Clarkson et al., 1991, Nature 352: 624-628.

A “variant” is a polypeptide that differs from a reference polypeptide by single or multiple non-native amino acid substitutions, deletions, and/or additions. In some embodiments, a variant retains at least one biological activity of the reference polypeptide. In some embodiments, a variant has a biological activity that the reference polypeptide substantially lacks. A “canine Fc region variant” comprises an amino acid sequence which differs from that of a wild type canine Fc region by at least one amino acid modification, preferably one or more amino acid substitution(s). Preferably, the canine Fc region variant has at least one amino acid substitution compared to a wild type canine Fc region, e.g., from one to ten amino acid substitutions, and preferably from one to five amino acid substitutions in a wild type canine Fc region. The canine Fc region variant herein will preferably possess at least 80% homology with a wild type canine Fc region, and most preferably at least 90% homology therewith, more preferably at least 95% homology therewith. In some embodiments, the canine Fc region is a canine IgGA Fc region variant, a canine IgGB Fc region variant, a canine IgGC Fc region variant, or a canine IgGD Fc region variant. A “feline Fc region variant” comprises an amino acid sequence which differs from that of a wild type feline Fc region by at least one amino acid modification, preferably one or more amino acid substitution(s). Preferably, the feline Fc region variant has at least one amino acid substitution compared to a wild type feline Fc region, e.g., from one to ten amino acid substitutions, and preferably from one to five amino acid substitutions in a wild type feline Fc region. The feline Fc region variant herein will preferably possess at least 80% homology with a wild type feline Fc region, and most preferably at least 90% homology therewith, more preferably at least 95% homology therewith. In some embodiments, the feline IgG Fc region is a feline IgG1a Fc region variant, a feline IgG1b Fc region variant, or a feline IgG2 Fc region variant. In some embodiments, the Fc region variant (e.g., a canine or feline Fc region variant) comprises the hinge, CH2, and CH3, but does not comprise CH1 or CL. In some embodiments, the Fc region variant (e.g., a canine or feline Fc region variant) comprises CH2 and CH3, but does not comprise CH1, the hinge, or CL. In some embodiments, the Fc region variant (e.g., a canine or feline Fc region variant) comprises CH1, hinge, CH2, and CH3, with or without CL. In some embodiments, the Fc region variant (e.g., a canine or feline Fc region variant) comprises CH1, hinge, CH2, CH3, and CL. For example, CL may be linked to CH1 via a disulfide bridge.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors can direct the expression of nucleic acids to which they are operatively linked. Such vectors are referred to herein as “expression vectors.”

As used herein, “administering” is meant a method of giving a dosage of a compound (e.g., a bispecific binding agent of the present disclosure, e.g., a bispecific antibody or an Fc construct) or a composition (e.g., a pharmaceutical composition, e.g., a pharmaceutical composition including a bispecific binding agent of the present disclosure) to a subject. The compositions utilized in the methods described herein can be administered, for example, parenterally, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The administration may be local or systemic. The method of administration can vary depending on various factors (e.g., the compound or composition being administered and the severity of the condition, disease, or disorder being treated).

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

### Bispecific Binding Agents

### Bispecific Antibodies:

The present disclosure provides a bispecific antibody comprising:
(a) a first binding domain that binds to a first antigen, wherein the first binding domain is linked to a first companion animal Fc region variant; and
(b) a second binding domain that binds to a second antigen, wherein the second binding domain is linked to a second companion animal Fc region variant. In some embodiments, at least one of the first binding domain and the second binding domain comprises a single-domain antibody.

For example, the present disclosure provides a bispecific antibody comprising:
(a) a first binding domain that binds to a first antigen, wherein the first binding domain is linked to a first companion animal Fc region variant; and
(b) a second binding domain that binds to a second antigen, wherein the second binding domain is linked to a second companion animal Fc region variant;
   wherein at least one of the first binding domain and the second binding domain comprises a single-domain antibody. In some examples, the first binding domain comprises a single-domain antibody. In some examples, the second binding domain comprises a single-domain antibody. In some examples, the first binding domain and the second binding domain each comprises a single-domain antibody.

The first binding domain or the second binding domain may bind to any suitable antigen(s). In some embodiments, the first binding domain and the second binding domain each specifically binds to an antigen independently selected from the group consisting of NGF, TrKA, ADAMTS, IL-1, IL-2, IL-4, IL-4R, Angiotensin type 1 (AT1) receptor, Angiotensin type 2 (AT2) receptor, IL-5, IL-12, IL-13, IL-31, IL-31R, IL-33, CD3, CD20, CD47, CD52, and complement system complex.

In some embodiments, the first binding domain and/or the second binding domain comprises an antibody, an antibody fragment, or a ligand-binding portion of a receptor. In some examples, the first binding domain or the second binding domain comprises an antibody, an antibody fragment, or a ligand-binding portion of a receptor. In some examples, the first binding domain and the second binding domain each comprises an antibody, an antibody fragment, or a ligand-binding portion of a receptor. In some embodiments, the antibody fragment is selected from the group consisting of Fab, single chain variable fragment (scFv), Fv, Fab′, Fab′-SH, F(ab′)₂, and diabody.

In some embodiments, the single-domain antibody is linked to the first companion animal Fc region variant or the second companion animal Fc region variant either directly or via a peptide linker. In some examples, the single-domain antibody is linked to the first companion animal Fc region variant directly. In some examples, the single-domain antibody is linked to the first companion animal Fc region variant via a peptide linker. In some examples, the single-domain antibody is linked to the second companion animal Fc region variant directly. In some examples, the single-domain antibody is linked to the second companion animal Fc region variant via a peptide linker.

In some embodiments, the single-domain antibody is a VHH domain. In one embodiment, the VHH domain comprises a C-terminal residue and the first companion animal Fc region variant or the second companion animal Fc region variant comprises an N-terminal residue, and the C-terminal residue of the VHH domain is linked either directly or via a peptide linker to the N-terminal residue of the first companion animal Fc region variant or the second companion animal Fc region variant. In another embodiment, the VHH domain comprises an N-terminal residue, and the first companion animal Fc region variant or the second companion animal Fc region variant comprises a C-terminal residue, and the N-terminal residue of the VHH domain is linked either directly or via a peptide linker to the C-terminal residue of the first companion animal Fc region variant or the second companion animal Fc region variant.

In some embodiments, the peptide linker comprises an amino acid sequence selected from the group consisting of:

(SEQ ID NO: 38)
(a) GPGGQ;
(SEQ ID NO: 363)
(b) PKRENGRVPRPPDCPKCP;
(SEQ ID NO: 364)
(c) VPKRENGRVPRPPDCPKCP;
(SEQ ID NO: 22)
(d) FNECRCTDTPPCPVPEP;
(SEQ ID NO: 23)
(e) PKRENGRVPRPPDCPKCPAPEM;
(SEQ ID NO: 24)
(f) AKECECKCNCNNCPCPGCGL;
(SEQ ID NO: 25)
(g) PKESTCKCISPCPVPES;
(SEQ ID NO: 26)
(h) PKESTCKCIPPCPVPES;
(SEQ ID NO: 27)
(i) KTDHPPGPKPCDCPKCP;
and
(SEQ ID NO: 29)
(j) KTASTIESKTGEGPKCP.

In one embodiment, the first companion animal Fc region variant and the second companion animal Fc region variant are canine Fc region variants. In some embodiments, the first canine Fc region variant or the second canine Fc region variant comprises an amino acid sequence that is at least 95% identical to an amino acid sequence selected from the group consisting of SEQ ID NOs: 9 to 12. In some embodiments, the first canine Fc region variant or the second canine Fc region variant comprises an amino acid sequence that is at least 95% identical to SEQ ID NO: 9. In some embodiments, the first canine Fc region variant or the second canine Fc region variant comprises an amino acid sequence that is at least 95% identical to SEQ ID NO: 10. In some embodiments, the first canine Fc region variant or the second canine Fc region variant comprises an amino acid sequence that is at least 95% identical to SEQ ID NO: 11. In some embodiments, the first canine Fc region variant or the second canine Fc region variant comprises an amino acid sequence that is at least 95% identical to SEQ ID NO: 12.

In some embodiments, the first canine Fc region variant and the second canine Fc region variant comprise complementary dimerization selectivity modules that promote dimerization between the first canine Fc region variant and the second canine Fc region variant. Any suitable complementary dimerization selectivity modules may be used.

In one embodiment, the first canine Fc region variant and the second canine Fc region variant each comprises a protuberance or a cavity, wherein if the first canine Fc region comprises a protuberance, the second canine Fc region comprises a cavity, and wherein if the first canine Fc region comprises a cavity, the second canine Fc region comprises a protuberance.

In some embodiments, the first canine Fc region variant and the second canine Fc region variant comprise amino acid substitutions selected from the group consisting of:
(a) S354C and T366W in the first canine Fc region variant; and Y349C, T366S, L368A, and Y407V in the second canine Fc region variant;
(b) T366W in the first canine Fc region variant; and T366S, L368A, and Y407V in the second canine Fc region variant;
(c) R392D and K409D in the first canine Fc region variant; and E356K and D399K in the second canine Fc region variant;
(d) S364H and F405A in the first canine Fc region variant; and Y349T and T394F in the second canine Fc region variant;
(e) F405L in the first canine Fc region variant; and K409R in the second canine Fc region variant; (f) T366L, R392L, and T394W in the first canine Fc region variant; and L351Y, F405A, and Y407V in the second canine Fc region variant;
(g) K360E and K409W in the first canine Fc region variant; and S347R, D399V, and F405T in the second canine Fc region variant;
(h) Y349C, K360E, and K409W in the first canine Fc region variant; and S347R, S354C, D399V, and F405T in the second canine Fc region variant;
(i) K370E and K409W in the first canine Fc region variant; and E357N, D399V, and F405T in the second canine Fc region variant;
(j) K360D, D399M, and Y407A in the first canine Fc region variant; and Q345R, S347R, T366V, and K409V in the second canine Fc region variant;
(k) Y349S, T366M, K370Y, and K409V in the first canine Fc region variant; and E356G, E357D, S364Q, and Y407A in the second canine Fc region variant;
(l) L351 D and L368E in the first canine Fc region variant; and L351K and T366K in the second canine Fc region variant;
(m) L368D and K370S in the first canine Fc region variant; and E356Q and S364K in the second canine Fc region variant; or
(n) T366Y in the first canine Fc region variant; and T366S, L368A, and Y407T in the second canine Fc region variant;
   wherein the amino acid positions are based on EU numbering.

In some embodiments, the first canine Fc region variant comprises amino acid substitutions S354C and T366W, and the second canine Fc region variant comprises amino acid substitutions T366S, L368A, Y407V, and Y349C.

In some embodiments, the first canine Fc region variant comprises amino acid substitution T366W, and the second canine Fc region variant comprises amino acid substitutions T366S, L368A, and Y407V.

In some embodiments, the first canine Fc region variant comprises amino acid substitutions R392D and K409D, and the second canine Fc region variant comprises amino acid substitutions E356K and D399K.

In some embodiments, the first canine Fc region variant comprises amino acid substitutions S364H and F405A, and the second canine Fc region variant comprises amino acid substitutions Y349T and T394F.

In some embodiments, the first canine Fc region variant comprises amino acid substitution F405L, and the second canine Fc region variant comprises amino acid substitution K409R.

In some embodiments, the first canine Fc region variant comprises amino acid substitutions T366L, R392L, and T394W, and the second canine Fc region variant comprises amino acid substitutions L351Y, F405A, and Y407V.

In some embodiments, the first canine Fc region variant comprises amino acid substitutions K360E and K409W, and the second canine Fc region variant comprises amino acid substitutions S347R, D399V, and F405T.

In some embodiments, the first canine Fc region variant comprises amino acid substitutions Y349C, K360E, and K409W, and the second canine Fc region variant comprises amino acid substitution S354C.

In some embodiments, the first canine Fc region variant comprises amino acid substitutions K370E and K409W, and the second canine Fc region variant comprises amino acid substitutions E357N, D399V, and F405T.

In some embodiments, the first canine Fc region variant comprises amino acid substitutions K360D, D399M, and Y407A, and the second canine Fc region variant comprises amino acid substitutions Q345R, S347R, T366V, and K409V.

In some embodiments, the first canine Fc region variant comprises amino acid substitutions Y349S, T366M, K370Y, and K409V, and the second canine Fc region variant comprises amino acid substitutions E356G, E357D, S364Q, and Y407A.

In some embodiments, the first canine Fc region variant comprises amino acid substitutions L351 D and L368E, and the second canine Fc region variant comprises amino acid substitutions L351K and T366K.

In some embodiments, the first canine Fc region variant comprises amino acid substitutions L368D and K370S, and the second canine Fc region variant comprises amino acid substitutions E356Q and S364K.

In some embodiments, the first canine Fc region variant comprises amino acid substitution T366Y, and the second canine Fc region variant comprises amino acid substitutions T366S, L368A, and Y407T.

In another embodiment, the first canine Fc region variant comprises a first charged region and the second canine Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first canine Fc region variant and the second canine Fc region variant further comprise CH1 domains comprising the following amino acid substitutions:
S183D in the first canine Fc region variant; and S183K in the second canine Fc region variant.

In some embodiments, the first canine Fc region variant and the second canine Fc region variant comprise CH3 domains comprising amino acid substitutions selected from the group consisting of:
(a) K409D in the first canine Fc region variant and D399K in the second canine Fc region variant;
(b) K390D and K409D in the first canine Fc region variant; and E356K and D399K in the second canine Fc region variant;
(c) K390D and K409D in the first canine Fc region variant; and E357K and D399K in the second canine Fc region variant; and
(d) K370D and K409D and in the first canine Fc region variant; and E357K and D399K in the second canine Fc region variant.

In some embodiments, the first canine Fc region variant comprises amino acid substitution K409D, and the second canine Fc region variant comprises amino acid substitution D399K.

In some embodiments, the first canine Fc region variant comprises amino acid substitutions K390D and K409D, and the second canine Fc region variant comprises amino acid substitutions E356K and D399K.

In some embodiments, the first canine Fc region variant comprises amino acid substitutions K390D and K409D, and the second canine Fc region variant comprises amino acid substitutions E357K and D399K.

In some embodiments, the first canine Fc region variant comprises amino acid substitutions K370D and K409D, and the second canine Fc region variant comprises amino acid substitutions E357K and D399K.

In some embodiments, the first canine Fc region variant and the second canine Fc region variant further comprise CL domains comprising the following amino acid substitutions:
S176K in the first canine Fc region variant; and S176D in the second canine Fc region variant.

In some embodiments, the first canine Fc region variant and/or the second canine Fc region variants further comprise at least one amino acid substitutions that increase half-life, including any amino acid substitutions as disclosed in U.S. Patent Application Publication Nos. 2020/0216536 and 2020/0362035, U.S. patent application Ser. No. 17/875,934, and U.S. Pat. No. 11,434,276, each of which is incorporated herein by reference in its entirety.

In some embodiments, the first canine Fc region variant and/or the second canine Fc region variants further comprise at least one of the following amino acid substitutions:
(a) 252Y and, optionally, at least one amino acid substitution selected from the group consisting of 251 D or 251E; 285N or 285D; 286D; 307Q; 308P; 315D; 430A or 430K; 433K; 435Y; and 436H;
(b) 252M and, optionally, at least one amino acid substitution selected from the group consisting of 251 D or 251E; 256D or 256F; 285N or 285D; 286D; 307Q; 308P; 315D; 430A or 430K; 433K; 435Y; and 436H;
(c) 434R;
(d) 426Y and, optionally, at least one amino acid substitution selected from the group consisting of 286F, 286W, 286L, or 286Y; 312P; 434R; and 436H;
(e) 426H and, optionally, at least one amino acid substitution selected from the group consisting of 286F, 286W, 286L, or 286Y; 312P; 434R; and 436H;
(f) 426F and, optionally, at least one amino acid substitution selected from the group consisting of 286F, 286W, 286L, or 286Y; 312P; 434R; and 426H; and
(g) 434R and, optionally, at least one amino acid substitution selected from the group consisting of 286L; 286Y; 312P; and 436H;
   wherein the amino acid positions are based on EU numbering.

In another embodiment, the first companion animal Fc region variant and the second companion animal Fc region variant are feline Fc region variants. In some embodiments, the first feline Fc region variant or the second feline Fc region variant comprises an amino acid sequence that is at least 95% identical to an amino acid sequence selected from the group consisting of SEQ ID NOs: 19 to 21. In some embodiments, the first feline Fc region variant or the second feline Fc region variant comprises an amino acid sequence that is at least 95% identical to SEQ ID NO: 19. In some embodiments, the first feline Fc region variant or the second feline Fc region variant comprises an amino acid sequence that is at least 95% identical to SEQ ID NO: 20. In some embodiments, the first feline Fc region variant or the second feline Fc region variant comprises an amino acid sequence that is at least 95% identical to SEQ ID NO: 21.

In some embodiments, the first feline Fc region variant and the second feline Fc region variant comprise complementary dimerization selectivity modules that promote dimerization between the first feline Fc region variant and the second feline Fc region variant.

In one embodiment, the first feline Fc region variant and the second feline Fc region variant each comprises a protuberance or a cavity, wherein if the first feline Fc region comprises a protuberance, the second feline Fc region comprises a cavity, and wherein if the first feline Fc region comprises a cavity, the second feline Fc region comprises a protuberance.

In some embodiments, the first feline Fc region variant and the second feline Fc region variant comprise amino acid substitutions selected from the group consisting of:
(a) T366W in the first feline Fc region variant; and T366S, L368A, and Y407V in the second feline Fc region variant;
(b) T366W in the first feline Fc region variant; and T366S, L368A, and Y398T in the second feline Fc region variant;
(c) A354C and T366W in the first feline Fc region variant; and Y349C, T366S, L368A, and Y407V in the second feline Fc region variant;
(d) R392D and K409D in the first feline Fc region variant; and E356K and D399K in the second feline Fc region variant;
(e) S364H and F405A in the first feline Fc region variant; and Y349T and T394F in the second feline Fc region variant;
(f) F405L in the first feline Fc region variant; and K409R in the second feline Fc region variant;
(g) T366L, R392L, and T394W in the first feline Fc region variant; and L351Y, F405A, and Y407V in the second feline Fc region variant;
(h) R360E and K409W in the first feline Fc region variant; and Q347R, D399V, and F405T in the second feline Fc region variant;
(i) Y349C, R360E, and K409W in the first feline Fc region variant; and Q347R, A354C, D399V, F405T in the second feline Fc region variant;
(j) K370E and K409W in the first feline Fc region variant; and E357N, D399V, and F405T in the second feline Fc region variant;
(k) R360D, D399M, and Y407A in the first feline Fc region variant; and E345R, Q347R, T366V, and K409V in the second feline Fc region variant;
(l) Y349S, K370Y, T366M, and K409V in the first feline Fc region variant; and E356G, E357D, S364Q, and Y407A in the second feline Fc region variant;
(m) L351 D and L368E in the first feline Fc region variant; and L351K and T366K in the second feline Fc region variant;
(n) L368D and K370S in the first feline Fc region variant; and E356Q and S364K in the second feline Fc region variant; and
(o) T366Y in the first feline Fc region variant; and T366S, L368A, and Y407T in the second feline Fc region variant;
   wherein the amino acid positions are based on EU numbering.

In some embodiments, the first feline Fc region variant comprises amino acid substitution T366W, and the second feline Fc region variant comprises amino acid substitutions T366S, L368A, and Y407V.

In some embodiments, the first feline Fc region variant comprises amino acid substitution T366W, and the second feline Fc region variant comprises amino acid substitutions T366S, L368A, and Y398T.

In some embodiments, the first feline Fc region variant comprises amino acid substitutions A354C and T366W, and the second feline Fc region variant comprises amino acid substitutions Y349C, T366S, L368A, and Y407V.

In some embodiments, the first feline Fc region variant comprises amino acid substitutions R392D and K409D, and the second feline Fc region variant comprises amino acid substitutions E356K and D399K.

In some embodiments, the first feline Fc region variant comprises amino acid substitutions S364H and F405A, and the second feline Fc region variant comprises amino acid substitutions Y349T and T394F.

In some embodiments, the first feline Fc region variant comprises amino acid substitution F405L, and the second feline Fc region variant comprises amino acid substitution K409R.

In some embodiments, the first feline Fc region variant comprises amino acid substitutions T366L, R392L, and T394W, and the second feline Fc region variant comprises amino acid substitutions L351 Y, F405A, and Y407V.

In some embodiments, the first feline Fc region variant comprises amino acid substitutions R360E and K409W, and the second feline Fc region variant comprises amino acid substitutions Q347R, D399V, and F405T.

In some embodiments, the first feline Fc region variant comprises amino acid substitutions Y349C, R360E, and K409W, and the second feline Fc region variant comprises amino acid substitutions Q347R, A354C, D399V, F405T.

In some embodiments, the first feline Fc region variant comprises amino acid substitutions K370E and K409W, and the second feline Fc region variant comprises amino acid substitutions E357N, D399V, and F405T.

In some embodiments, the first feline Fc region variant comprises amino acid substitutions R360D, D399M, and Y407A, and the second feline Fc region variant comprises amino acid substitutions E345R, Q347R, T366V, and K409V.

In some embodiments, the first feline Fc region variant comprises amino acid substitutions Y349S, K370Y, T366M, and K409V, and the second feline Fc region variant comprises amino acid substitutions E356G, E357D, S364Q, and Y407A.

In some embodiments, the first feline Fc region variant comprises amino acid substitutions L351 D and L368E, and the second feline Fc region variant comprises amino acid substitutions L351K and T366K.

In some embodiments, the first feline Fc region variant comprises amino acid substitutions L368D and K370S, and the second feline Fc region variant comprises amino acid substitutions E356Q and S364K.

In some embodiments, the first feline Fc region variant comprises amino acid substitution T366Y, and the second feline Fc region variant comprises amino acid substitutions T366S, L368A, and Y407T.

In another embodiment, the first feline Fc region variant comprises a first charged region and the second feline Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first feline Fc region variant and the second feline Fc region variant further comprise CH1 domains comprising the following amino acid substitutions:
S183D in the first feline Fc region variant; and S183K in the second feline Fc region variant.

In some embodiments, the first feline Fc region variant and the second feline Fc region variant comprise CH3 domains comprising amino acid substitutions selected from the group consisting of: (a) K409D in the first feline Fc region variant and D399K in the second feline Fc region variant; and (b) K370D and K409D and in the first feline Fc region variant; and E357K and D399K in the second feline Fc region variant.

In some embodiments, the first feline Fc region variant comprises amino acid substitution K409D, and the second feline Fc region variant comprises amino acid substitution D399K.

In some embodiments, the first feline Fc region variant comprises amino acid substitutions K370D and K409D, and the second feline Fc region variant comprises amino acid substitutions E357K and D399K.

In some embodiments, the first feline Fc region variant and the second feline Fc region variant further comprise CL domains comprising the following amino acid substitutions: S176K in the first feline Fc region variant; and S176D in the second feline Fc region variant.

In some embodiments, the first feline Fc region variant and/or the second feline Fc region variants further comprise at least one amino acid substitutions that increase half-life, including any amino acid substitutions as disclosed in U.S. Patent Application Publication No. 2022/0259282, U.S. patent application Ser. No. 18/046,082, and U.S. Pat. No. 11,498,953, each of which is incorporated herein by reference in its entirety.

In some embodiments, the first and/or second feline Fc region variants further comprise at least one of the following amino acid substitutions:
(a) at least one amino acid substitution selected from the group consisting of 286E; 311V; and 428Y;
(b) two or more amino acid substitutions selected from the group consisting of 286E; 311V; and 428Y; and
(c) 286E, 311V, and 428Y;
   wherein the amino acid positions are based on EU numbering.

### Fc Constructs:

The present disclosure also provides an Fc construct comprising:
(a) a first polypeptide comprising a first companion animal Fc region variant; and
(b) a second polypeptide comprising a second companion animal Fc region variant, wherein the first companion animal Fc region variant and the second companion animal Fc region variant comprise complementary dimerization selectivity modules that promote dimerization between the first companion animal Fc region variant and the second companion animal Fc region variant. In some embodiments, the first polypeptide or the second polypeptide does not comprise an antibody.

For example, the present disclosure also provides an Fc construct comprising:
(a) a first polypeptide comprising a first companion animal Fc region variant; and
(b) a second polypeptide comprising a second companion animal Fc region variant, wherein the first companion animal Fc region variant and the second companion animal Fc region variant comprise complementary dimerization selectivity modules that promote dimerization between the first companion animal Fc region variant and the second companion animal Fc region variant, and wherein the first polypeptide or the second polypeptide does not comprise an antibody.

In some embodiments, the Fc construct further comprises a protein selected from the group consisting of EPO, CTLA4, LFA3, VEGFR1, VEGFR3, IL-1R, IL-4R, GLP-1 receptor agonist, and thrombopoietin binding peptide.

In one embodiment, the first companion animal Fc region variant and the second companion animal Fc region variant are canine Fc region variants. In some embodiments, the first canine Fc region variant or the second canine Fc region variant comprises an amino acid sequence that is at least 95% identical to an amino acid sequence selected from the group consisting of SEQ ID NOs: 9 to 12. In some embodiments, the first canine Fc region variant or the second canine Fc region variant comprises an amino acid sequence that is at least 95% identical to SEQ ID NO: 9. In some embodiments, the first canine Fc region variant or the second canine Fc region variant comprises an amino acid sequence that is at least 95% identical to SEQ ID NO: 10. In some embodiments, the first canine Fc region variant or the second canine Fc region variant comprises an amino acid sequence that is at least 95% identical to SEQ ID NO: 11. In some embodiments, the first canine Fc region variant or the second canine Fc region variant comprises an amino acid sequence that is at least 95% identical to SEQ ID NO: 12.

In one embodiment, the first canine Fc region variant and the second canine Fc region variant each comprises a protuberance or a cavity, wherein if the first canine Fc region comprises a protuberance, the second canine Fc region comprises a cavity, and wherein if the first canine Fc region comprises a cavity, the second canine Fc region comprises a protuberance.

In some embodiments, the first canine Fc region variant and the second canine Fc region variant each comprises amino acid substitutions selected from the group consisting of:
(a) S354C and T366W in the first canine Fc region variant; and T366S, L368A, Y407V, and Y349C in the second canine Fc region variant;
(b) T366W in the first canine Fc region variant; and T366S, L368A, and Y407V in the second canine Fc region variant;
(c) R392D and K409D in the first canine Fc region variant; and E356K and D399K in the second canine Fc region variant;
(d) S364H and F405A in the first canine Fc region variant; and Y349T and T394F in the second canine Fc region variant;
(e) F405L in the first canine Fc region variant; and K409R in the second canine Fc region variant;
(f) T366L, R392L, and T394W in the first canine Fc region variant; and L351Y, F405A, and Y407V in the second canine Fc region variant;
(g) K360E and K409W in the first canine Fc region variant; and S347R, D399V, and F405T in the second canine Fc region variant;
(h) Y349C, K360E, and K409W in the first canine Fc region variant; and S347R, S354C, D399V, and F405T in the second canine Fc region variant;
(i) K370E and K409W in the first canine Fc region variant; and E357N, D399V, and F405T in the second canine Fc region variant;
(j) K360D, D399M, and Y407A in the first canine Fc region variant; and Q345R, S347R, T366V, and K409V in the second canine Fc region variant;
(k) Y349S, T366M, K370Y, and K409V in the first canine Fc region variant; and E356G, E357D, S364Q, and Y407A in the second canine Fc region variant;
(l) L351 D and L368E in the first canine Fc region variant; and L351K and T366K in the second canine Fc region variant;
(m) L368D and K370S in the first canine Fc region variant; and E356Q and S364K in the second canine Fc region variant; or
(n) T366Y in the first canine Fc region variant; and T366S, L368A, and Y407T in the second canine Fc region variant;
   wherein the amino acid positions are based on EU numbering.

In some embodiments, the first canine Fc region variant comprises amino acid substitutions T366W and S354C, and the second canine Fc region variant comprises amino acid substitutions T366S, L368A, Y407V, and Y349C.

In some embodiments, the first canine Fc region variant comprises amino acid substitution T366W, and the second canine Fc region variant comprises amino acid substitutions T366S, L368A, and Y407V.

In some embodiments, the first canine Fc region variant comprises amino acid substitutions K409D and R392D, and the second canine Fc region variant comprises amino acid substitutions D399K and E356K.

In some embodiments, the first canine Fc region variant comprises amino acid substitutions S364H and F405A, and the second canine Fc region variant comprises amino acid substitutions Y349T and T394F.

In some embodiments, the first canine Fc region variant comprises amino acid substitution F405L, and the second canine Fc region variant comprises amino acid substitution K409R.

In some embodiments, the first canine Fc region variant comprises amino acid substitutions T366L, R392L, and T394W, and the second canine Fc region variant comprises amino acid substitutions L351Y, F405A, and Y407V.

In some embodiments, the first canine Fc region variant comprises amino acid substitutions K360E and K409W, and the second canine Fc region variant comprises amino acid substitutions S347R, D399V, and F405T.

In some embodiments, the first canine Fc region variant comprises amino acid substitutions K360E, K409W, and Y349C, and the second canine Fc region variant comprises amino acid substitution S354C.

In some embodiments, the first canine Fc region variant comprises amino acid substitutions K370E and K409W, and the second canine Fc region variant comprises amino acid substitutions E357N, D399V, and F405T.

In some embodiments, the first canine Fc region variant comprises amino acid substitutions K360D, D399M, and Y407A, and the second canine Fc region variant comprises amino acid substitutions Q345R, S347R, T366V, and K409V.

In some embodiments, the first canine Fc region variant comprises amino acid substitutions Y349S, K370Y, T366M, and K409V, and the second canine Fc region variant comprises amino acid substitutions E356G, E357D, S364Q, and Y407A.

In some embodiments, the first canine Fc region variant comprises amino acid substitutions L351 D and L368E, and the second canine Fc region variant comprises amino acid substitutions L351K and T366K.

In some embodiments, the first canine Fc region variant comprises amino acid substitutions L368D and K370S, and the second canine Fc region variant comprises amino acid substitutions E356Q and S364K.

In some embodiments, the first canine Fc region variant comprises amino acid substitution T366Y, and the second canine Fc region variant comprises amino acid substitutions T366S, L368A, and Y407T.

In another embodiment, the first canine Fc region variant comprises a first charged region and the second canine Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first canine Fc region variant and the second canine Fc region variant further comprise CH1 domains comprising the following amino acid substitutions:
S183D in the first canine Fc region variant; and S183K in the second canine Fc region variant.

In some embodiments, the first canine Fc region variant and the second canine Fc region variant comprise CH3 domains comprising amino acid substitutions selected from the group consisting of:
(a) K409D in the first canine Fc region variant and D399K in the second canine Fc region variant;
(b) K390D and K409D in the first canine Fc region variant; and E356K and D399K in the second canine Fc region variant;
(c) K390D and K409D in the first canine Fc region variant; and E357K and D399K in the second canine Fc region variant; and
(d) K370D and K409D and in the first canine Fc region variant; and E357K and D399K in the second canine Fc region variant.

In some embodiments, the first canine Fc region variant comprises amino acid substitution K409D, and the second canine Fc region variant comprises amino acid substitution D399K.

In some embodiments, the first canine Fc region variant comprises amino acid substitutions K390D and K409D, and the second canine Fc region variant comprises amino acid substitutions E356K and D399K.

In some embodiments, the first canine Fc region variant comprises amino acid substitutions K390D and K409D, and the second canine Fc region variant comprises amino acid substitutions E357K and D399K.

In some embodiments, the first canine Fc region variant comprises amino acid substitutions K370D and K409D, and the second canine Fc region variant comprises amino acid substitutions E357K and D399K.

In some embodiments, the first canine Fc region variant and the second canine Fc region variant further comprise CL domains comprising the following amino acid substitutions:
S176K in the first canine Fc region variant; and S176D in the second canine Fc region variant.

In some embodiments, the first canine Fc region variant and/or the second canine Fc region variants further comprise at least one amino acid substitutions that increase half-life, including any amino acid substitutions as disclosed in U.S. Patent Application Publication Nos. 2020/0216536 and 2020/0362035, U.S. patent application Ser. No. 17/875,934, and U.S. Pat. No. 11,434,276, each of which is incorporated herein by reference in its entirety.

In some embodiments, the first canine Fc region variant and the second canine Fc region variant further comprise at least one of the following amino acid substitutions:
(a) 252Y and, optionally, at least one amino acid substitution selected from the group consisting of 251 D or 251E; 285N or 285D; 286D; 307Q; 308P; 315D; 430A or 430K; 433K; 435Y; and 436H;
(b) 252M and, optionally, at least one amino acid substitution selected from the group consisting of 251 D or 251E; 256D or 256F; 285N or 285D; 286D; 307Q; 308P; 315D; 430A or 430K; 433K; 435Y; and 436H;
(c) 434R;
(d) 426Y and, optionally, at least one amino acid substitution selected from the group consisting of 286F, 286W, 286L, or 286Y; 312P; 434R; and 436H;
(e) 426H and, optionally, at least one amino acid substitution selected from the group consisting of 286F, 286W, 286L, or 286Y; 312P; 434R; and 436H;
(f) 426F and, optionally, at least one amino acid substitution selected from the group consisting of 286F, 286W, 286L, or 286Y; 312P; 434R; and 426H; and
(g) 434R and, optionally, at least one amino acid substitution selected from the group consisting of 286L; 286Y; 312P; and 436H;
   wherein the amino acid positions are based on EU numbering.

In another embodiment, the first companion animal Fc region variant and the second companion animal Fc region variant are feline Fc region variants. In some embodiments, the first feline Fc region variant or the second feline Fc region variant comprises an amino acid sequence that is at least 95% identical to an amino acid sequence selected from the group consisting of SEQ ID NOs: 19 to 21. In some embodiments, the first feline Fc region variant or the second feline Fc region variant comprises an amino acid sequence that is at least 95% identical to SEQ ID NO: 19. In some embodiments, the first feline Fc region variant or the second feline Fc region variant comprises an amino acid sequence that is at least 95% identical to SEQ ID NO: 20. In some embodiments, the first feline Fc region variant or the second feline Fc region variant comprises an amino acid sequence that is at least 95% identical to SEQ ID NO: 21.

In one embodiment, the first feline Fc region variant and the second feline Fc region variant each comprises a protuberance or a cavity, wherein if the first feline Fc region comprises a protuberance, the second feline Fc region comprises a cavity, and wherein if the first feline Fc region comprises a cavity, the second feline Fc region comprises a protuberance.

In some embodiments, the first feline Fc region variant and the second feline Fc region variant comprise amino acid substitutions selected from the group consisting of:
(a) T366W in the first feline Fc region variant; and T366S, L368A, and Y407V in the second feline Fc region variant;
(b) T366W in the first feline Fc region variant; and T366S, L368A, and Y398T in the second feline Fc region variant;
(c) A354C and T366W in the first feline Fc region variant; and Y349C, T366S, L368A, and Y407V in the second feline Fc region variant;
(d) R392D and K409D in the first feline Fc region variant; and E356K and D399K in the second feline Fc region variant;
(e) S364H and F405A in the first feline Fc region variant; and Y349T and T394F in the second feline Fc region variant;
(f) F405L in the first feline Fc region variant; and K409R in the second feline Fc region variant;
(g) T366L, R392L, and T394W in the first feline Fc region variant; and L351Y, F405A, and Y407V in the second feline Fc region variant;
(h) R360E and K409W in the first feline Fc region variant; and Q347R, D399V, and F405T in the second feline Fc region variant;
(i) Y349C, R360E, and K409W in the first feline Fc region variant; and Q347R, A354C, D399V, F405T in the second feline Fc region variant;
(j) K370E and K409W in the first feline Fc region variant; and E357N, D399V, and F405T in the second feline Fc region variant;
(k) R360D, D399M, and Y407A in the first feline Fc region variant; and E345R, Q347R, T366V, and K409V in the second feline Fc region variant;
(l) Y349S, K370Y, T366M, and K409V in the first feline Fc region variant; and E356G, E357D, S364Q, and Y407A in the second feline Fc region variant;
(m) L351 D and L368E in the first feline Fc region variant; and L351K and T366K in the second feline Fc region variant;
(n) L368D and K370S in the first feline Fc region variant; and E356Q and S364K in the second feline Fc region variant; and
(o) T366Y in the first feline Fc region variant; and T366S, L368A, and Y407T in the second feline Fc region variant;
   wherein the amino acid positions are based on EU numbering.

In some embodiments, the first feline Fc region variant comprises amino acid substitution T366W, and the second feline Fc region variant comprises amino acid substitutions T366S, L368A, and Y407V.

In some embodiments, the first feline Fc region variant comprises amino acid substitution T366W, and the second feline Fc region variant comprises amino acid substitutions T366S, L368A, and Y398T.

In some embodiments, the first feline Fc region variant comprises amino acid substitutions A354C and T366W, and the second feline Fc region variant comprises amino acid substitutions Y349C, T366S, L368A, and Y407V.

In some embodiments, the first feline Fc region variant comprises amino acid substitutions R392D and K409D, and the second feline Fc region variant comprises amino acid substitutions E356K and D399K.

In some embodiments, the first feline Fc region variant comprises amino acid substitutions S364H and F405A, and the second feline Fc region variant comprises amino acid substitutions Y349T and T394F.

In some embodiments, the first feline Fc region variant comprises amino acid substitution F405L, and the second feline Fc region variant comprises amino acid substitution K409R.

In some embodiments, the first feline Fc region variant comprises amino acid substitutions T366L, R392L, and T394W, and the second feline Fc region variant comprises amino acid substitutions L351 Y, F405A, and Y407V.

In some embodiments, the first feline Fc region variant comprises amino acid substitutions R360E and K409W, and the second feline Fc region variant comprises amino acid substitutions Q347R, D399V, and F405T.

In some embodiments, the first feline Fc region variant comprises amino acid substitutions Y349C, R360E, and K409W, and the second feline Fc region variant comprises amino acid substitutions Q347R, A354C, D399V, F405T.

In some embodiments, the first feline Fc region variant comprises amino acid substitutions K370E and K409W, and the second feline Fc region variant comprises amino acid substitutions E357N, D399V, and F405T.

In some embodiments, the first feline Fc region variant comprises amino acid substitutions R360D, D399M, and Y407A, and the second feline Fc region variant comprises amino acid substitutions E345R, Q347R, T366V, and K409V.

In some embodiments, the first feline Fc region variant comprises amino acid substitutions Y349S, K370Y, T366M, and K409V, and the second feline Fc region variant comprises amino acid substitutions E356G, E357D, S364Q, and Y407A.

In some embodiments, the first feline Fc region variant comprises amino acid substitutions L351 D and L368E, and the second feline Fc region variant comprises amino acid substitutions L351K and T366K.

In some embodiments, the first feline Fc region variant comprises amino acid substitutions L368D and K370S, and the second feline Fc region variant comprises amino acid substitutions E356Q and S364K.

In some embodiments, the first feline Fc region variant comprises amino acid substitution T366Y, and the second feline Fc region variant comprises amino acid substitutions T366S, L368A, and Y407T.

In another embodiment, the first feline Fc region variant comprises a first charged region and the second feline Fc region variant comprises a second charged region, and wherein the first charged region forms a charge pair with the second charged region. In some embodiments, the first charged region comprises a basic amino acid residue and the second charged region comprises an acidic amino acid residue.

In some embodiments, the first feline Fc region variant and the second feline Fc region variant further comprise CH1 domains comprising the following amino acid substitutions:
S183D in the first feline Fc region variant; and S183K in the second feline Fc region variant.

In some embodiments, the first feline Fc region variant and the second feline Fc region variant comprise CH3 domains comprising amino acid substitutions selected from the group consisting of:
(a) K409D in the first feline Fc region variant and D399K in the second feline Fc region variant; and
(b) K370D and K409D and in the first feline Fc region variant; and E357K and D399K in the second feline Fc region variant.

In some embodiments, the first feline Fc region variant comprises amino acid substitution K409D, and the second feline Fc region variant comprises amino acid substitution D399K.

In some embodiments, the first feline Fc region variant comprises amino acid substitutions K370D and K409D, and the second feline Fc region variant comprises amino acid substitutions E357K and D399K.

In some embodiments, the first feline Fc region variant and the second feline Fc region variant further comprise CL domains comprising the following amino acid substitutions:
S176K in the first feline Fc region variant; and S176D in the second feline Fc region variant.

In some embodiments, the first feline Fc region variant and/or the second feline Fc region variants further comprise at least one amino acid substitutions that increase half-life, including any amino acid substitutions as disclosed in U.S. Patent Application Publication No. 2022/0259282, U.S. patent application Ser. No. 18/046,082, and U.S. Pat. No. 11,498,953, each of which is incorporated herein by reference in its entirety.

In some embodiments, the first feline Fc region variant and the second feline Fc region variant further comprise at least one of the following amino acid substitutions:
(a) at least one amino acid substitution selected from the group consisting of 286E; 311V; and 428Y;
(d) two or more amino acid substitutions selected from the group consisting of 286E; 311V; and 428Y; and
(c) 286E, 311V, and 428Y;
   wherein the amino acid positions are based on EU numbering.

### Binding Domains:

In some embodiments, the bispecific binding agents (e.g., bispecific antibodies and Fc constructs) described herein include an antibody hinge region. The hinge region may be placed between the antigen or ligand-binding domain and the Fc region variant. In some embodiments, the hinge region is attached to the C-terminus of a cytokine, a growth factor, an enzyme, or a peptide and the hinge region is attached to the N-terminus of the Fc region variant. Exemplary hinge region sequences for canine antibodies are provided below:

IgGA:
(SEQ ID NO: 22)
FNECRCTDTPPCPVPEP;
IgGB:
(SEQ ID NO: 23)
PKRENGRVPRPPDCPKCPAPEM;
IgGC:
(SEQ ID NO: 24)
AKECECKCNCNNCPCPGCGL;
IgGD:
(SEQ ID NO: 25)
PKESTCKCISPCPVPES;
and
IgGDmut:
(SEQ ID NO: 26)
PKESTCKCIPPCPVPES.

Exemplary hinge region sequences for feline antibodies are provided below:

IgG1a:
(SEQ ID NO: 27)
KTDHPPGPKPCDCPKCP;
IgG1b:
(SEQ ID NO: 28)
KTDHPPGPKPCDCPKCP;
and
IgG2:
(SEQ ID NO: 29)
KTASTIESKTGEGPKCP;

The hinge region, if used, in a bispecific binding agent of this disclosure may include zero to six (i.e., 0, 1, 2, 3, 4, 5, or 6) amino acid substitutions relative to an amino acid sequence set forth in any one of SEQ ID NOs: 22-29. In some embodiments, the hinge region used in a recombinant protein of this disclosure is at least 80%, at least 85%, at least 90%, at least 95%, at least 96%, at least 97%, at least 98%, at least 99%, or 100% identical to an amino acid sequence set forth in any one of SEQ ID NOs: 22-29.

In some embodiments, a linker sequence may be used instead of an antibody hinge sequence to connect a polypeptide or polypeptides (e.g., antibodies, single-domain antibodies, ligand-binding domains of receptors, enzymes, ligands, peptides) to the companion animal (e.g., canine or feline) Fc region variants disclosed herein. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Examples of peptide linkers include: Gly, Ser; Gly Ser; Gly Gly Ser; Ser Gly Gly; Gly Gly Gly Ser (SEQ ID NO: 30); Ser Gly Gly Gly (SEQ ID NO: 31); Gly Gly Gly Gly Ser (SEQ ID NO: 32); Ser Gly Gly Gly Gly (SEQ ID NO: 33); Gly Gly Gly Gly Gly Ser (SEQ ID NO: 34); Ser Gly Gly Gly Gly Gly (SEQ ID NO: 35); Gly Gly Gly Gly Gly Gly Ser (SEQ ID NO: 36); Ser Gly Gly Gly Gly Gly Gly (SEQ ID NO: 37); Gly Pro Gly Gly Gln (SEQ ID NO: 38); (Gly Gly Gly Gly Ser)ₙ (SEQ ID NO: 32), wherein n is an integer of one or more (e.g., 1, 2, 3, 4, 5); and (Ser Gly Gly Gly Gly)ₙ (SEQ ID NO: 33), wherein n is an integer of one or more (e.g., 1, 2, 3, 4, 5).

Non-peptide linkers may also be used to link a polypeptide or polypeptides of interest to an Fc region variant disclosed herein. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, and the like.

The bispecific binding agent (e.g., bispecific antibody or Fc construct) of this disclosure may comprise a binding domain. The binding domain can specifically bind to a protein, subunit, domain, motif, and/or epitope of a selected target described herein. In some embodiments, the binding domain comprises a single-domain antibody. In some embodiments, the single-domain antibody is a VHH domain. In some embodiments, the binding domain comprises an antibody, an antibody fragment, or a ligand-binding portion of a receptor. In some embodiments, the antibody or the antibody fragment comprises six complementarity determining regions (CDRs) of an immunoglobulin molecule. In other embodiments, the antibody fragment is selected from the group consisting of Fab, single chain variable fragment (scFv), Fv, Fab′, Fab′-SH, F(ab′)₂, nanobody, and diabody. In other embodiments, the ligand-binding portion of a receptor comprises a ligand binding domain of a receptor protein or an extracellular domain of a receptor protein. In some embodiments, a polypeptide or polypeptides (e.g., fusion polypeptide) can comprise a protein, wherein the protein is a therapeutic protein described herein. In some embodiments, the target (e.g., for the target of the binding domain) or the therapeutic protein (e.g., for the fusion polypeptide) is selected from the group consisting of: 17-IA, 4-1 BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, IgE, Angiotensin type 1 (AT1) receptor, Angiotensin type 2 (AT2) receptor, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD47, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxini, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, GLP1, GLP2, Glucagon, Glut 4, glycoprotein IIb/IIIa (GP IIb/IIIa), GM-CSF, gp130, gp72, GRO, GnRH, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, cardiac myosin, cytomegalovirus (CMV), growth hormone (GH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NAV 1.7, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, oncostatin M receptor (OSMR), OPG, OPN, OSM, OX40L, OX40R, p150, p95, PADPr, Parathyroid hormone, PARC, PARP, PBR, PBSF, PCAD, P-Cadherin, PCNA, PD1, PDL1, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4 DcR2, TRUNDD), TNFRSF11 A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40p50), TNFRSF6 (Fas Apo-1, APT1, CD95), TNFRSF6B (DcR3M68, TR6), TNFRSF7 (CD27), TNFRSF8 (CD30), TNFRSF9 (4-1 BB CD137, ILA), TNFRSF21 (DR6), TNFRSF22 (DCTRAIL R2 TNFRH2), TNFRST23 (DCTRAIL R1TNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk (e.g., TrkA), TROP-2, TSG, TSLP, tumor-associated antigen CA 125, tumor-associated antigen expressing Lewis Y related carbohydrate, TWEAK, TXB2, Ung, UPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factor.

In some embodiments, the antibody or the antibody fragment comprises one or more complementarity determining regions (CDRs) with amino acid sequences selected from Table 2 below. For example, the antibody or the antibody fragment may include a CDR-H1, a CDR-H2, a CDR-H3, a CDR-L1, a CDR-L2, and a CDR-L3 selected from Table 2. For example, the antibody or the antibody fragment may include all six CDRs for an antibody that is listed as binding to a particular target in Table 2. In some embodiments, the antibody or antibody fragment may be any antibody or antibody fragment disclosed in U.S. Patent Application Publication Nos. US 2020/0062840, US 2022/0251209, US 2021/0040223, US 2022/0204615, US 2022/0251230, US 2021/0163618, US 2021/0253722, US 2022/0119513, US 2022/0106391, or US 2022/0177594; U.S. Pat. Nos. 11,091,556, 11,447,561, 10,040,849, or U.S. Pat. No. 9,951,128; and International Patent Application Publication Nos. WO 2020/056393, WO 2022/079138, WO 2021/123092, or WO 2022/029447 A1.

TABLE 2
Exemplary CDR sequences for canine antibodies

| Target | Publication Number | Sequences | SEQ ID NO: |
|---|---|---|---|
| IL-31 | U.S. Pat. No. 2020/0062840 | CDR-H1: GDSITSGYW | 39 |
| | | CDR-H2: YISYSGITDYNPSLKS | 40 |
| | | CDR-H3: ARYGNYGYAMDY | 41 |
| | | CDR-L1: RASESVDTYGNSFMH | 42 |
| | | CDR-L2: RASNLES | 43 |
| | | CDR-L3: QQSYEDPWT | 44 |
| IL-31 | U.S. Pat. No. 2020/0062840 | CDR-H1: GDSITSGYW | 39 |
| | | CDR-H2: YISYSGITYYNPSLKS | 45 |
| | | CDR-H3: ARYGNYGYAMDY | 41 |
| | | CDR-L1: RASESVDTYGNSFIH | 46 |
| | | CDR-L2: RASNLES | 43 |
| | | CDR-L3: QQSYEDPWT | 44 |
| IL-31 | U.S. Pat. No. 2020/0062840 | CDR-H1: GDSITSGYW | 39 |
| | | CDR-H2: YISYSGITDYNPSLKS | 40 |
| | | CDR-H3: ARYGNYGYAMDY | 41 |
| | | CDR-L1: RASESVDTYGNSFMH | 46 |
| | | CDR-L2: RASNLES | 43 |
| | | CDR-L3: QQSYEDPWT | 44 |
| IL-4R | WO 2020/056393 | CDR-H1: GYTFTSYVMH | 47 |
| | | CDR-H2: YINPKNDGTFYNGKFKG | 48 |
| | | CDR-H3: FNYGIAY | 49 |
| | | CDR-L1: RASQEISGYLS | 50 |
| | | CDR-L2: AASTLDS | 51 |
| | | CDR-L3: VQYASYPWT | 52 |
| IL-4R | WO 2020/056393 | CDR-H1: GYTFTSYVMH | 47 |
| | | CDR-H2: YINPNNDGTFYNGKFKG | 53 |
| | | CDR-H3: FYYGFAY | 54 |
| | | CDR-L1: RASQEISGYLS | 50 |
| | | CDR-L2: AASTLDS | 51 |
| | | CDR-L3: LQYASYPWT | 55 |
| IL-4R | WO 2020/056393 | CDR-H1: TVSGFSLSRYSVH | 56 |
| | | CDR-H2: GMIWGGGST | 57 |
| | | CDR-H3: TYRGYALDY | 58 |
| | | CDR-L1: RASQDISNYLN | 59 |
| | | CDR-L2: YYTSRLQS | 60 |
| | | CDR-L3: QQANTLPLT | 61 |
| IL-4R | WO 2020/056393 | CDR-H1: AASGFTFSSFGMH | 62 |
| | | CDR-H2: AYIRSDSSTIY | 63 |
| | | CDR-H3: SGYYGSFSLTY | 64 |
| | | CDR-L1: SASSSVSSNYLH | 65 |
| | | CDR-L2: YRTSNLPS | 66 |
| | | CDR-L3: QQGSGMLT | 67 |
| IL-31R | WO 2022/079138 | CDR-H1: NYYMA | 68 |
| | | CDR-H2: SISTGGGNTYYRDSVKG | 69 |
| | | CDR-H3: HGTLYFDY | 70 |
| | | CDR-L1: ERSSGDIGDSYVS | 71 |
| | | CDR-L2: VDDQRPS | 72 |
| | | CDR-L3: QSYDSNIDGPV | 73 |
| IL-31R | WO 2022/079138 | CDR-H1: DYYMA | 74 |
| | | CDR-H2: TISYDGSSTYYRDSVRG | 75 |
| | | CDR-H3: GPLTDWAPNWFAY | 76 |
| | | CDR-L1: QTSEDIYSGLA | 77 |
| | | CDR-L2: GASRLED | 78 |
| | | CDR-L3: QQGLKYPNT | 79 |
| IL-31R | WO 2022/079138 | CDR-H1: SNGVS | 80 |
| | | CDR-H2: AISSGGSTYYNSVLKS | 81 |
| | | CDR-H3: RLSGYNYVPFAY | 82 |
| | | CDR-L1: KASQNIYKHLA | 83 |
| | | CDR-L2: NANSLQT | 84 |
| | | CDR-L3: QQYYSGDT | 85 |
| IL-4R | U.S. Pat. No. 11,091,556 | CDR-H1: DYAMT | 86 |
| | | CDR-H2: SISGSGGNTYYADSVKG | 87 |
| | | CDR-H3: DRLSITIRPRYYGLDV | 88 |
| | | CDR-L1: RSSQSLLYSIGYNYLD | 89 |
| | | CDR-L2: LGSNRAS | 90 |
| | | CDR-L3: MQALQTPYT | 91 |
| PD-L1 | U.S. Pat. No. 11,447,561 | CDR-H1: SYAMS | 92 |
| | | CDR-H2: TISDGGSYTHYPDNLMG | 93 |
| | | CDR-H3: ESYDGYYVAN | 94 |
| | | CDR-L1: RASQSISNNLH | 95 |
| | | CDR-L2: YASQSIS | 96 |
| | | CDR-L3: QQSNSWPQT | 97 |
| PD-L1 | U.S. Pat. No. 11,447,561 | CDR-H1: DYYMN | 98 |
| | | CDR-H2: WIFPGSGATYYNERFMG | 99 |
| | | CDR-H3: SDWDVGDF | 100 |
| | | CDR-L1: RSSRSLLHTNGITYLS | 101 |
| | | CDR-L2: QMSNLAS | 102 |
| | | CDR-L3: AQTLGLPRT | 103 |
| CTLA-4 | U.S. Pat. No. 2022/0251209 | CDR-H1: DYNMD | 104 |
| | | CDR-H2: NINPNSESTSYNQKFKG | 105 |
| | | CDR-H3: DGNRYDAWFAY | 106 |
| | | CDR-L1: SASSSVTYMH | 107 |
| | | CDR-L2: STSILAS | 108 |
| | | CDR-L3: QQRTSYPLT | 109 |
| IL-4R | U.S. Pat. No. 2021/0040223 | CDR-H1: NYWIH | 110 |
| | | CDR-H2: RIDPNSGGTKYNEKFKS | 111 |
| | | CDR-H3: FGSTYGFAY | 112 |
| | | CDR-L1: KSSQSLLNSRTRKNYLA | 113 |
| | | CDR-L2: WASTRES | 114 |
| | | CDR-L3: KQSYNLYT | 115 |
| IL-4R | U.S. Pat. No. 2022/0204615 | CDR-H1: GYTFTTYGMS | 116 |
| | | CDR-H2: WINIYSGIPTYADDFKG | 117 |
| | | CDR-H3: FDGPDY | 118 |
| | | CDR-L1: KSSQSLLNSVNQKNYLA | 119 |
| | | CDR-L2: FASARVS | 120 |
| | | CDR-L3: QQYFSTPLT | 121 |
| IL-4R | U.S. Pat. No. 2022/0204615 | CDR-H1: GFNIKNTYMH | 122 |
| | | CDR-H2: RIAPANVDTKYAPKFQG | 123 |
| | | CDR-H3: IYYDYDGDIDV | 124 |
| | | CDR-L1: HASQNINVWLS | 125 |
| | | CDR-L2: KASHLHT | 126 |
| | | CDR-L3: QQGQSWPLT | 127 |
| IL-4R | U.S. Pat. No. 2022/0204615 | CDR-H1: GYTFTRYNMH | 128 |
| | | CDR-H2: TIYPGYGDTSYNQKFKG | 129 |
| | | CDR-H3: EFADDYPIPPFDY | 130 |
| | | CDR-L1: RSSQNIVHSNGNTYLE | 131 |
| | | CDR-L2: KVSNRFS | 132 |
| | | CDR-L3: FQGSHVPYT | 133 |
| IL-4R | U.S. Pat. No. 2022/0204615 | CDR-H1: GFSLTSYGVH | 134 |
| | | CDR-H2: WINIYSGIPTYADDFKG | 117 |
| | | CDR-H3: FDGPDY | 118 |
| | | CDR-L1: KSSQSLLNSVNQKNYLA | 119 |
| | | CDR-L2: FASTRVS | 135 |
| | | CDR-L3: QQYFSTPLT | 121 |
| IL-4R | U.S. Pat. No. 2022/0204615 | CDR-H1: GYTFTTYGMS | 116 |
| | | CDR-H2: WINIYSGIPTYADDFKG | 117 |
| | | CDR-H3: FDGPDY | 118 |
| | | CDR-L1: KSSQSLLNSVNQKNYLA | 119 |
| | | CDR-L2: FASTRVS | 135 |
| | | CDR-L3: QQYFSTPLT | 121 |
| IL-4R | U.S. Pat. No. 2022/0204615 | CDR-H1: GFNIKNTYMH | 122 |
| | | CDR-H2: RIDPANVNTKYAPKFQG | 136 |
| | | CDR-H3: IFYDYDGDIDV | 137 |
| | | CDR-L1: HASQNINVWLS | 125 |
| | | CDR-L2: KASNLHT | 138 |
| | | CDR-L3: QQGQSYPLT | 139 |
| IL-4R | U.S. Pat. No. 2022/0204615 | CDR-H1: GYTFTTYGMS | 116 |
| | | CDR-H2: WINIYSGMPTYADDFKG | 140 |
| | | CDR-H3: FDGPDY | 118 |
| | | CDR-L1: KSSQSLLNSVNQKNYLA | 119 |
| | | CDR-L2: FASTRIS | 141 |
| | | CDR-L3: QQYFSTPLT | 121 |
| IL-22 | WO 2021/123092 | CDR-H1: NYYMH | 142 |
| | | CDR-H2: WINPYTGSAFYAQKFRG | 143 |
| | | CDR-H3: EPEKFDSDDSDV | 144 |
| | | CDR-L1: TGSSSNIGAGYGVH | 145 |
| | | CDR-L2: GDSNRPS | 146 |
| | | CDR-L3: QSYDNSLSGYV | 147 |
| OX40L | WO 2022/029447 | CDR-H1: GFTFSSY | 148 |
| | | CDR-H2: NTGGTV | 149 |
| | | CDR-H3: CARGYGVFDYW | 150 |
| | | CDR-L1: GGDNIGSKSVH | 151 |
| | | CDR-L2: YDSRRPT | 152 |
| | | CDR-L3: CQVWDNSDRASWVF | 153 |
| OX40L | WO 2022/029447 | CDR-H1: GGSVTSSH | 154 |
| | | CDR-H2: TGG | 155 |
| | | CDR-H3: CARGGGYSGTWKDYYVMDYW | 156 |
| | | CDR-L1: GGDNLGSKSVH | 157 |
| | | CDR-L2: FDTSRPT | 158 |
| | | CDR-L3: CQVWDSSAKASVF | 159 |
| OX40L | WO 2022/029447 | CDR-H1: GFTFSNF | 160 |
| | | CDR-H2: NSGGFN | 161 |
| | | CDR-H3: CARDWDTHLDTNWFYYW | 162 |
| | | CDR-L1: RGDNIGSKSVH | 163 |
| | | CDR-L2: YDSSRPT | 164 |
| | | CDR-L3: CQVWDISAKASVF | 165 |
| OX40L | WO 2022/029447 | CDR-H1: GFTFSYY | 166 |
| | | CDR-H2: NSGGFS | 167 |
| | | CDR-H3: CASESRWGDSYSGMTYW | 168 |
| | | CDR-L1: GGDNIGSKSVH | 151 |
| | | CDR-L2: YDNSRPT | 169 |
| | | CDR-L3: CQVWDSSAKASVF | 159 |
| OX40L | WO 2022/029447 | CDR-H1: GFTFSNY | 170 |
| | | CDR-H2: TSDGIV | 171 |
| | | CDR-H3: CASGLFLVVGGGTFW | 172 |
| | | CDR-L1: GGDNIGSKSVH | 151 |
| | | CDR-L2: SDSSRPT | 173 |
| | | CDR-L3: CQVWDSSAKASVF | 159 |
| OX40L | WO 2022/029447 | CDR-H1: GFTFSSY | 148 |
| | | CDR-H2: ASGGTV | 174 |
| | | CDR-H3: CTRWKGGTFGYGMDYW | 175 |
| | | CDR-L1: GGDNIGSKSVH | 151 |
| | | CDR-L2: YDNNRPA | 176 |
| | | CDR-L3: CQVWDSSAKASVF | 159 |
| OX40L | WO 2022/029447 | CDR-H1: GFTFSNY | 170 |
| | | CDR-H2: TNDGIV | 177 |
| | | CDR-H3: CASGLFLVVGGGTFW | 172 |
| | | CDR-L1: GGDNIGSKSVH | 151 |
| | | CDR-L2: YDSSRPT | 164 |
| | | CDR-L3: CQVWDSSAKASVF | 159 |
| OX40L | WO 2022/029447 | CDR-H1: GFTFSSN | 178 |
| | | CDR-H2: SGDGIY | 179 |
| | | CDR-H3: CATGIYPNAFGYW | 180 |
| | | CDR-L1: GGDNIGSKSVH | 151 |
| | | CDR-L2: YDSSRPT | 164 |
| | | CDR-L3: CQVWDSSANVF | 181 |
| OX40L | WO 2022/029447 | CDR-H1: GFTFSSY | 148 |
| | | CDR-H2: SSGGTF | 182 |
| | | CDR-H3: CASGLFLVVGGGNYW | 183 |
| | | CDR-L1: GGDNIGSKSVH | 151 |
| | | CDR-L2: SDSSRPT | 173 |
| | | CDR-L3: CQVWDSSASVF | 184 |
| OX40L | WO 2022/029447 | CDR-H1: GFTFSNY | 170 |
| | | CDR-H2: NSDGRV | 185 |
| | | CDR-H3: CARWRGGTFGYGMDYW | 186 |
| | | CDR-L1: GGDNIGSKSVH | 151 |
| | | CDR-L2: YDSSRPT | 164 |
| | | CDR-L3: CQVWDSSAKASVF | 159 |
| OX40L | WO 2022/029447 | CDR-H1: GFTFSNY | 170 |
| | | CDR-H2: NSDGRI | 187 |
| | | CDR-H3: CARWRGGTFGYGMDYW | 186 |
| | | CDR-L1: GGDNIGSKSVH | 151 |
| | | CDR-L2: YDSSRPT | 164 |
| | | CDR-L3: CQVWDSSAKASVF | 159 |
| OX40L | WO 2022/029447 | CDR-H1: GFTFHNY | 188 |
| | | CDR-H2: SSDGRF | 189 |
| | | CDR-H3: CANGLFLVLGGENYW | 190 |
| | | CDR-L1: GGDNIGSKSVH | 151 |
| | | CDR-L2: YDSSRPT | 164 |
| | | CDR-L3: CQVWDSSASVF | 184 |
| OX40L | WO 2022/029447 | CDR-H1: GFTFSSN | 178 |
| | | CDR-H2: SGDGSF | 191 |
| | | CDR-H3: CASGIYPNAFGYW | 192 |
| | | CDR-L1: GGDNIGSKSVH | 151 |
| | | CDR-L2: YDSSRPT | 164 |
| | | CDR-L3: CQVRDSSANVF | 193 |
| CD20 | U.S. Pat. No. 2022/0251230 | CDR-H1: DYGML | 194 |
| | | CDR-H2: YISSGSSTIYYADRVKG | 195 |
| | | CDR-H3: GTFAY | 196 |
| | | CDR-L1: RSSQSLIYNNGNTYLH | 197 |
| | | CDR-L2: KVSNRFS | 132 |
| | | CDR-L3: SQSTHVPFT | 198 |
| CD20 | U.S. Pat. No. 2022/0251230 | CDR-H1: DDYMH | 199 |
| | | CDR-H2: WIDPENGHTKYASKFQG | 200 |
| | | CDR-H3: LRHYYGSSYVSPHYY | 201 |
| | | CDR-L1: KASQNVGPNVA | 202 |
| | | CDR-L2: SASYRYS | 203 |
| | | CDR-L3: QQYNNYPYT | 204 |
| CD20 | U.S. Pat. No. 2022/0251230 | CDR-H1: DYYMN | 98 |
| | | CDR-H2: DINPNNGDTSYNQKFKG | 205 |
| | | CDR-H3: GGVLRYPYYYVMDY | 206 |
| | | CDR-L1: RSNKSLLHRNGNTYLY | 207 |
| | | CDR-L2: RMSNLAS | 208 |
| | | CDR-L3: MQHLEFPFT | 209 |
| CD20 | U.S. Pat. No. 2021/0163618 | CDR-H1: KGSGRTFTSYNMH | 210 |
| | | CDR-H2: WVRQMPGKGLEWMG | 211 |
| | | CDR-H3: AIYPLTGDTSYNQKSKL | 212 |
| | | CDR-L1: RASRSVPYIH | 213 |
| | | CDR-L2: YATSALAS | 214 |
| | | CDR-L3: QQWLSNPPT | 215 |
| NGF | U.S. Pat. No. 10,040,849 | CDR-H1: NNNVN | 216 |
| | | CDR-H2: GVWAGGATDYNSALKS | 217 |
| | | CDR-H3: DGGYSSSTLYAMDA | 218 |
| | | CDR-L1: RASEDIYNALA | 219 |
| | | CDR-L2: NTDTLHT | 220 |
| | | CDR-L3: QHYFHYPRT | 221 |
| NGF | U.S. Pat. No. 9,951,128 | CDR-H1: LIGYDIN | 222 |
| | | CDR-H2: MIWGDGTTDYNSALKS | 223 |
| | | CDR-H3: GGYYYGTSYYFDY | 224 |
| | | CDR-L1: RASQDISNHLN | 225 |
| | | CDR-L2: YISRFHS | 226 |
| | | CDR-L3: QQSKTLPYT | 227 |
| NGF | U.S. Pat. No. 2021/0253722 | CDR-H1: TNNIGILG | 228 |
| | | CDR-H2: GNG | 229 |
| | | CDR-H3: QSFDTTLGAHV | 230 |
| | | CDR-L1: GFTFSSHG | 231 |
| | | CDR-L2: INSGGSST | 232 |
| | | CDR-L3: AKESVGGVVEQLVGPHFDY | 233 |
| NGF | U.S. Pat. No. 2021/0253722 | CDR-H1: TMDIDIFG | 234 |
| | | CDR-H2: SDG | 235 |
| | | CDR-H3: QSGDSTLGALAI | 236 |
| | | CDR-L1: GFTFSTYG | 237 |
| | | CDR-L2: ISSGGSST | 238 |
| | | CDR-L3: AGSRYTYAYGGGYEFHF | 239 |
| TGF-ß | U.S. Pat. No. 2022/0119513 | CDR-H1: SSWMN | 240 |
| | | CDR-H2: QIYPGDGDTNYNGKFKG | 241 |
| | | CDR-H3: ARHYDGSTDY | 242 |
| | | CDR-L1: RASENIYSNLA | 243 |
| | | CDR-L2: AATNLAD | 244 |
| | | CDR-L3: QHFWGTPYT | 245 |
| TGF-ß | U.S. Pat. No. 2022/0119513 | CDR-H1: FSSYGMH | 246 |
| | | CDR-H2: VISYDGSIKYY | 247 |
| | | CDR-H3: TGEYSGYDTDPQYS | 248 |
| | | CDR-L1: RASQGIGDDLG | 249 |
| | | CDR-L2: GTSTLQS | 250 |
| | | CDR-L3: LQDSNYPLT | 251 |
| TGF-ß | U.S. Pat. No. 2022/0119513 | CDR-H1: GYIFITY | 252 |
| | | CDR-H2: FPASGS | 253 |
| | | CDR-H3: GDGNYALDAMDY | 254 |
| | | CDR-L1: RASESVDSYGNSFMH | 255 |
| | | CDR-L2: LASNLES | 256 |
| | | CDR-L3: QQNNEDPLT | 257 |
| NGF | U.S. Pat. No. 2022/0106391 | CDR-H1: GFTLTQYG | 258 |
| | | CDR-H2: VIWATGATD | 259 |
| | | CDR-H3: DGWWYATSWYFDV | 260 |
| | | CDR-L1: KASQDINHYLN | 261 |
| | | CDR-L2: YTSRLHS | 262 |
| | | CDR-L3: QQGDHFPRT | 263 |
| OSMR-ß | U.S. Pat. No. 2022/0177594 | CDR-H1: SYAMS | 92 |
| | | CDR-H2: YISSGGDYIYYADTVKG | 264 |
| | | CDR-H3: DPITGTFAY | 265 |
| | | CDR-L1: RASQDINNYLN | 266 |
| | | CDR-L2: YTSTLHS | 267 |
| | | CDR-L3: QQGNTLPWT | 268 |
| OSMR-ß | U.S. Pat. No. 2022/0177594 | CDR-H1: SYAMS | 92 |
| | | CDR-H2: YISSGGDYFYYADTVKG | 269 |
| | | CDR-H3: DPITGTFAY | 265 |
| | | CDR-L1: RASQDITNYLN | 270 |
| | | CDR-L2: YTSTLHS | 267 |
| | | CDR-L3: QQGHMLPWT | 271 |
| OSMR-ß | U.S. Pat. No. 2022/0177594 | CDR-H1: NYWMN | 272 |
| | | CDR-H2: QIYPGHVNTNYNGNFKD | 273 |
| | | CDR-H3: SADNSGFVLFAY | 274 |
| | | CDR-L1: RASKSVSTSGYSYLH | 275 |
| | | CDR-L2: LASNLES | 256 |
| | | CDR-L3: QHSRELPLT | 276 |
| OSMR-ß | U.S. Pat. No. 2022/0177594 | CDR-H1: DYYMA | 74 |
| | | CDR-H2: NINYDGSSTYYLDSLKS | 277 |
| | | CDR-H3: GLTWDFDV | 278 |
| | | CDR-L1: KASQDVDTAVA | 279 |
| | | CDR-L2: LASTRHT | 280 |
| | | CDR-L3: QQYSRFPLT | 281 |

In some embodiments, the antibody or the antibody fragment comprises one or more complementarity determining regions (CDRs) with amino acid sequences selected from Table 3 below. For example, the antibody or the antibody fragment may include a CDR-H1, a CDR-H2, a CDR-H3, a CDR-L1, a CDR-L2, and a CDR-L3 selected from Table 3. For example, the antibody or the antibody fragment may include all six CDRs for an antibody that is listed as binding to a particular target in Table 3. In some embodiments, the antibody or antibody fragment may be any antibody or antibody fragment disclosed in U.S. Patent Application Publication Nos. US 2020/0062840, US 2022/0119513, US 2022/0106391, US 2022/0177594, or US 2022/0127351; U.S. Pat. No. 9,328,164; and International Patent Application Publication No. WO 2020/056393.

TABLE 3
Exemplary CDR sequences for feline antibodies

| Target | Publication Number | Sequences | SED ID NO: |
|---|---|---|---|
| IL-31 | U.S. Pat. No. 2020/0062840 | CDR-H1: GDSITSGYW | 282 |
| | | CDR-H2: YISYSGITDYNPSLKS | 283 |
| | | CDR-H3: ARYGNYGYAMDY | 284 |
| | | CDR-L1: RASESVDTYGNSFMH | 285 |
| | | CDR-L2: RASNLES | 286 |
| | | CDR-L3: QQSYEDPWT | 287 |
| IL-31 | U.S. Pat. No. 2020/0062840 | CDR-H1: GDSITSGYW | 282 |
| | | CDR-H2: YISYSGITYYNPSLKS | 288 |
| | | CDR-H3: ARYGNYGYAMDY | 284 |
| | | CDR-L1: RASESVDTYGNSFIH | 289 |
| | | CDR-L2: RASNLES | 286 |
| | | CDR-L3: QQSYEDPWT | 287 |
| IL-31 | U.S. Pat. No. 2020/0062840 | CDR-H1: GDSITSGYW | 282 |
| | | CDR-H2: YISYSGITDYNPSLKS | 283 |
| | | CDR-H3: ARYGNYGYAMDY | 284 |
| | | CDR-L1: RASESVDTYGNSFMH | 285 |
| | | CDR-L2: RASNLES | 286 |
| | | CDR-L3: QQSYEDPWT | 287 |
| IL-4R | WO 2020/056393 | CDR-H1: GYTFTSYVMH | 290 |
| | | CDR-H2: YINPKNDGTFYNGKFKG | 291 |
| | | CDR-H3: FNYGIAY | 292 |
| | | CDR-L1: RASQEISGYLS | 293 |
| | | CDR-L2: AASTLDS | 294 |
| | | CDR-L3: VQYASYPWT | 295 |
| IL-4R | WO 2020/056393 | CDR-H1: GYTFTSYVMH | 290 |
| | | CDR-H2: YINPNNDGTFYNGKFKG | 296 |
| | | CDR-H3: FYYGFAY | 297 |
| | | CDR-L1: RASQEISGYLS | 293 |
| | | CDR-L2: AASTLDS | 294 |
| | | CDR-L3: LQYASYPWT | 298 |
| TGF-ß | U.S. Pat. No. 2022/0119513 | CDR-H1: SSWMN | 299 |
| | | CDR-H2: QIYPGDGDTNYNGKFKG | 300 |
| | | CDR-H3: ARHYDGSTDY | 301 |
| | | CDR-L1: RASENIYSNLA | 302 |
| | | CDR-L2: AATNLAD | 303 |
| | | CDR-L3: QHFWGTPYT | 304 |
| TGF-ß | U.S. Pat. No. 2022/0119513 | CDR-H1: FSSYGMH | 305 |
| | | CDR-H2: VISYDGSIKYY | 306 |
| | | CDR-H3: TGEYSGYDTDPQYS | 307 |
| | | CDR-L1: RASQGIGDDLG | 308 |
| | | CDR-L2: GTSTLQS | 309 |
| | | CDR-L3: LQDSNYPLT | 310 |
| TGF-ß | U.S. Pat. No. 2022/0119513 | CDR-H1: GYIFITY | 311 |
| | | CDR-H2: FPASGS | 312 |
| | | CDR-H3: GDGNYALDAMDY | 313 |
| | | CDR-L1: RASESVDSYGNSFMH | 314 |
| | | CDR-L2: LASNLES | 315 |
| | | CDR-L3: QQNNEDPLT | 316 |
| NGF | U.S. Pat. No. 2022/0106391 | CDR-H1: GFTLTQYG | 317 |
| | | CDR-H2: VIWATGATD | 318 |
| | | CDR-H3: DGWWYATSWYFDV | 319 |
| | | CDR-L1: KASQDINHYLN | 320 |
| | | CDR-L2: YTSRLHS | 321 |
| | | CDR-L3: QQGDHFPRT | 322 |
| OSMR-ß | U.S. Pat. No. 2022/0177594 | CDR-H1: DYGMH | 323 |
| | | CDR-H2: YISSGSRAVFFADTVKG | 324 |
| | | CDR-H3: DRYDGRGFAY | 325 |
| | | CDR-L1: RASQSISNNLH | 326 |
| | | CDR-L2: YASQSIS | 327 |
| | | CDR-L3: QQSNSWPLT | 328 |
| OSMR-ß | U.S. Pat. No. 2022/0177594 | CDR-H1: SYAMS | 329 |
| | | CDR-H2: YISSGGDYIYYADTVKG | 330 |
| | | CDR-H3: DPITGTFAY | 331 |
| | | CDR-L1: RASQDINNYLN | 332 |
| | | CDR-L2: YTSTLHS | 333 |
| | | CDR-L3: QQGNTLPWT | 334 |
| OSMR-ß | U.S. Pat. No. 2022/0177594 | CDR-H1: SYAMS | 329 |
| | | CDR-H2: YISSGGDYFYYADTVKG | 335 |
| | | CDR-H3: DPITGTFAY | 331 |
| | | CDR-L1: RASQDITNYLN | 336 |
| | | CDR-L2: YTSTLHS | 333 |
| | | CDR-L3: QQGHMLPWT | 337 |
| OSMR-ß | U.S. Pat. No. 2022/0177594 | CDR-H1: DYYMA | 338 |
| | | CDR-H2: NINYDGSSTYYLDSLKS | 339 |
| | | CDR-H3: GLTWDFDV | 340 |
| | | CDR-L1: KASQDVDTAVA | 341 |
| | | CDR-L2: LASTRHT | 342 |
| | | CDR-L3: QQYSRFPLT | 343 |
| NGF | U.S. Pat. No. 9,328,164 | CDR-H1: NNNVN | 344 |
| | | CDR-H2: GVWAGGATDYNSALKGR | 345 |
| | | CDR-H3: DGGYSSSTLYAMDA | 346 |
| | | CDR-L1: RASEDIYNALA | 347 |
| | | CDR-L2: NTDTLHT | 348 |
| | | CDR-L3: HYFHYPRT | 349 |
| IL-31 | U.S. Pat. No. 2022/0127351 | CDR-H1: SYTIH | 350 |
| | | CDR-H2: NINPTSGYTENNQRFKD | 351 |
| | | CDR-H3: WGFKYDGEWSFDV | 352 |
| | | CDR-L1: RASQGISIWLS | 353 |
| | | CDR-L2: KASNLHI | 354 |
| | | CDR-L3: LQSQTYPLT | 355 |
| IL-31 | U.S. Pat. No. 2022/0127351 | CDR-H1: YYDIN | 356 |
| | | CDR-H2: WIFPGDGGTKYNETFKG | 357 |
| | | CDR-H3: ARGGTSVIRDAMDY | 358 |
| | | CDR-L1: RASESVDNYGISFMH | 359 |
| | | CDR-L2: RASNLES | 286 |
| | | CDR-L3: QQSNKDPLT | 360 |

In some embodiments, the binding domain specifically binds to one or more therapeutic targets or antigens in a companion animal (e.g., a canine or feline), such as, but are not limited to, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bp1, LFA-1, Neuronal growth factor (NGF), NGFR, NGF-beta, OSMR, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1 BB CD137, ILA), VEFGR-1 (flt-1), VEGF, VEGFR, and VEGFR-3 (flt-4).

In some embodiments, the bispecific binding agent can comprise a protein, wherein the protein is a therapeutic protein, e.g., EPO, CTLA4, LFA3, VEGFR1/VEGFR3, IL-1R, IL-4R, GLP-1 receptor agonist, or Thrombopoietin binding peptide. In some embodiments, the therapeutic protein is ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bp1, LFA-1, Neuronal growth factor (NGF), NGFR, NGF-beta, OSMR, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1 BB CD137, ILA), VEFGR-1 (flt-1), VEGF, VEGFR, or VEGFR-3 (flt-4).

In some embodiments, the therapeutic protein is any protein described herein. In one embodiment, the bispecific antibody or the Fc construct further comprises a modified canine IgG CH2 domain, IgG CH3 domain, or IgG Fc region as described herein. In another embodiment, the bispecific antibody or the Fc construct further comprises a modified feline IgG CH2 domain, IgG CH3 domain, or IgG Fc region as described herein. The modified canine or feline IgG CH2 domain, IgG CH3 domain, or IgG Fc region can enhance the half-life the therapeutic proteins in vivo.

### Canine Fc Regions

Dogs have four IgG heavy chains referred to as A, B, C, and D. These heavy chains represent four different subclasses of dog IgG, which are referred to as IgGA, IgGB, IgGC and IgGD. The amino acid and DNA sequences for these heavy chains are available from Tang et al., 2001, Vet. Immunol. Immunopathol., 80: 259-270 and the GENBANK database. For example, the amino acid sequence of IgGA heavy chain has GENBANK accession number AAL35301.1, IgGB has GENBANK accession number AAL35302.1, IgGC has GENBANK accession number AAL35303.1, and IgGD has GENBANK accession number AAL35304.1. Canine antibodies also include two types of light chains: kappa and lambda. The DNA and amino acid sequence of these light chains can also be obtained from GENBANK database. For example, the dog kappa light chain amino acid sequence has accession number ABY57289.1 and the dog lambda light chain has accession number ABY55569.1.

### CH2 Region of a Canine Fc Region:

The CH2 region of a canine antibody comprises or consists of amino acids 237 to 340 (according to EU numbering) of a canine IgG antibody. It is to be understood that the CH2 region may include one to six (e.g., 1, 2, 3, 4, 5, or 6) additional amino acids or deletions at their N and/or C-terminus.

The amino acid sequence of the CH2 region of canine IgGA is provided below:

(SEQ ID NO: 1)
GPSVLI FPPKPKDILR ITRTPEVTCV VLDLGREDPE
VQISWFVDGK EVHTAKTQSR EQQFNGTYRV VSVLPIEHQD
WLTGKEFKCR VNHIDLPSPI ERTISKAR

The amino acid sequence of the CH2 domain of canine IgGB is provided below:

(SEQ ID NO: 2)
GPSVFIFPPK PKDTLLIART PEVTCVVVDL DPEDPEVQIS
WFVDGKQMQT AKTQPREEQF NGTYRVVSVL PIGHQDWLKG
KQFTCKVNNK ALPSPIERTI SKAR

The amino acid sequence of the CH2 domain of canine IgGC is provided below:

(SEQ ID NO: 3)
GPSVFIFPP KPKDILVTAR TPTVTCVVVD LDPENPEVQI
SWFVDSKQVQ TANTQPREEQ SNGTYRVVSV LPIGHQDWLS
GKQFKCKVNN KALPSPIEEI ISKTP

The amino acid sequence of the CH2 domain of canine IgGD is provided below:

(SEQ ID NO: 4)
GPSV FIFPPKPKDI LRITRTPEIT CVVLDLGRED PEVQISWFVD
GKEVHTAKTQ PREQQFNSTY RVVSVLPIEH QDWLTGKEFK
CRVNHIGLPS PIERTISKAR

### CH3 Region of a Canine Fc Region:

The CH3 region of a canine antibody comprises or consists of amino acids 345 to 447 (according to EU numbering) of a canine IgG antibody. It is to be understood that the CH3 region may include one to six (e.g., 1, 2, 3, 4, 5, or 6) additional amino acids or deletions at their N and/or C-terminus.

The amino acid sequence of the CH3 domain of canine IgGA is provided below:

(SEQ ID NO: 5)
KPSVYVLP PSPKELSSSD TVSITCLIKD FYPPDIDVEW
QSNGQQEPER KHRMTPPQLD EDGSYFLYSK LSVDKSRWQQ
GDPFTCAVMH ETLQNHYTDL SLSHSPGK

The amino acid sequence of the CH3 domain of canine IgGB is provided below:

(SEQ ID NO: 6)
QP SVYVLPPSRE ELSKNTVSLT CLIKDFFPPD IDVEWQSNGQ
QEPESKYRTT PPQLDEDGSY FLYSKLSVDK SRWQRGDTFI
CAVMHEALHN HYTQESLSHS PGK

The amino acid sequence of the CH3 domain of canine IgGC is provided below:

(SEQ ID NO: 7)
Q PNVYVLPPSR DEMSKNTVTL TCLVKDFFPP EIDVEWQSNG
QQEPESKYRM TPPQLDEDGS YFLYSKLSVD KSRWQRGDTF
ICAVMHEALH NHYTQISLSH SPGK

The amino acid sequence of the CH3 domain of canine IgGD is provided below:

(SEQ ID NO: 8)
QPSVYV LPPSPKELSS SDTVTLTCLI KDFFPPEIDV
EWQSNGQPEP ESKYHTTAPQ LDEDGSYFLY SKLSVDKSRW
QQGDTFTCAV MHEALQNHYT DLSLSHSPGK

### Sequences of Canine Fc Regions:

The Fc region of a canine IgG antibody comprises or consists of amino acids 231 to 447 (according to EU numbering) of the canine IgG antibody.

The amino acid sequence of the Fc domain of canine IgGA is provided below:

(SEQ ID NO: 9)
VPEPLGGPSVLI FPPKPKDILR ITRTPEVTCV VLDLGREDPE
VQISWFVDGK EVHTAKTQSR EQQFNGTYRV VSVLPIEHQD
WLTGKEFKCR VNHIDLPSPI ERTISKARGR AHKPSVYVLP
PSPKELSSSD TVSITCLIKD FYPPDIDVEW QSNGQQEPER
KHRMTPPQLD EDGSYFLYSK LSVDKSRWQQ GDPFTCAVMH
ETLQNHYTDL SLSHSPGK

The amino acid sequence of the Fc domain of canine IgGB is provided below:

(SEQ ID NO: 10)
APEMLGGPSVFIFPPK PKDTLLIART PEVTCVVVDL DPEDPEVQIS
WFVDGKQMQT AKTQPREEQF NGTYRVVSVL PIGHQDWLKG
KQFTCKVNNK ALPSPIERTI SKARGQAHQP SVYVLPPSRE
ELSKNTVSLT CLIKDFFPPD IDVEWQSNGQ QEPESKYRTT
PPQLDEDGSY FLYSKLSVDK SRWQRGDTFI CAVMHEALHN
HYTQESLSHS PGK

The amino acid sequence of the Fc domain of canine IgGC is provided below:

(SEQ ID NO: 11)
GCGLLGGPSVFIFPP KPKDILVTAR TPTVTCVVVD LDPENPEVQI
SWFVDSKQVQ TANTQPREEQ SNGTYRVVSV LPIGHQDWLS
GKQFKCKVNN KALPSPIEEI ISKTPGQAHQ PNVYVLPPSR
DEMSKNTVTL TCLVKDFFPP EIDVEWQSNG QQEPESKYRM
TPPQLDEDGS YFLYSKLSVD KSRWQRGDTF ICAVMHEALH
NHYTQISLSH SPGK

The amino acid sequence of the Fc domain of canine IgGD is provided below:

(SEQ ID NO: 12)
VPESLGGPSV FIFPPKPKDI LRITRTPEIT CVVLDLGRED
PEVQISWFVD GKEVHTAKTQ PREQQFNSTY RVVSVLPIEH
QDWLTGKEFK CRVNHIGLPS PIERTISKAR GQAHQPSVYV
LPPSPKELSS SDTVTLTCLI KDFFPPEIDV EWQSNGQPEP
ESKYHTTAPQ LDEDGSYFLY SKLSVDKSRW QQGDTFTCAV
MHEALQNHYT DLSLSHSPGK

### Feline Fc Regions

Cats typically have three IgG heavy chains referred to as IgG1a, IgG1b, and IgG2. These heavy chains represent three different subclasses of cat IgG. The amino acid and DNA sequences for these heavy chains are available from Tang et al., 2001, Vet. Immunol. Immunopathol., 80: 259-270 and the GENBANK database. For example, the amino acid sequence of feline IgG1a heavy chain has GENBANK accession number BAA32229.1, feline IgG1b heavy chain has GENBANK accession number BAA32230.1, and feline IgG2 heavy chain has GENBANK accession number KF811175.1. Feline antibodies also include two types of light chains: kappa and lambda. The DNA and amino acid sequence of these light chains can also be obtained from GENBANK database. For example, the cat kappa light chain amino acid sequence has accession number AF198257.1 and the cat lambda light chain has accession number E07339.1.

### CH2 Region of a Feline Fc Region:

The CH2 region of a feline antibody comprises or consists of amino acids 231 to 340 (according to EU numbering) of a feline IgG antibody. It is to be understood that the CH2 region may include one to six (e.g., 1, 2, 3, 4, 5, or 6) additional amino acids or deletions at their N and/or C-terminus.

The amino acid sequence of the CH2 region of feline IgG1a is provided below:

(SEQ ID NO: 13)
PPEMLGGPSIFIFPPKPKDTLSISRTPEVTCLVVDLGPDDSDVQI
TWFVDNTQVYTAKTSPREEQFNSTYRVVSVLPILHQDWLKGKEFK
CKVNSKSLPSPIERTISKAK

The amino acid sequence of the CH2 domain of feline I G1 b is provided below:

(SEQ ID NO: 14)
PPEMLGGPSIFIFPPKPKDTLSISRTPEVTCLVVDLGPDDSDVQI
TWFVDNTQVYTAKTSPREEQFNSTYRVVSVLPILHQDWLKGKEFK
CKVNSKSLPSPIERTISKDK

The amino acid sequence of the CH2 domain of feline IgG2 is provided below:

(SEQ ID NO: 15)
VPEIPGAPSVFIFPPKPKDTLSISRTPEVTCLVVDLGPDDSNVQI
TWFVDNTEMHTAKTRPREEQFNSTYRVVSVLPILHQDWLKGKEFK
CKVNSKSLPSAMERTISKAK

### CH3 Region of a Feline Fc Region:

The CH3 region of a feline antibody comprises or consists of amino acids 341 to 447 (according to EU numbering) of a feline IgG antibody. It is to be understood that the CH3 region may include one to six (e.g., 1, 2, 3, 4, 5, 6) additional amino acids or deletions at their N and/or C-terminus.

The amino acid sequence of the CH3 domain of feline IgG1a is provided below:

(SEQ ID NO: 16)
GQPHEPQVYVLPPAQEELSRNKVSVTCLIKSFHPPDIAVEWEITG
QPEPENNYRTTPPQLDSDGTYFVYSKLSVDRSHWQRGNTYTCSVS
HEALHSHHTQKSLTQSPGK

The amino acid sequence of the CH3 domain of feline IgG1 b is provided below:

(SEQ ID NO: 17)
GQPHEPQVYVLPPAQEELSRNKVSVTCLIEGFYPSDIAVEWEITG
QPEPENNYRTTPPQLDSDGTYFLYSRLSVDRSRWQRGNTYTCSVS
HEALHSHHTQKSLTQSPGK

The amino acid sequence of the CH3 domain of feline IgG2 is provided below:

(SEQ ID NO: 18)
GQPHEPQVYVLPPTQEELSENKVSVTCLIKGFHPPDIAVEWEITG
QPEPENNYQTTPPQLDSDGTYFLYSRLSVDRSHWQRGNTYTCSVS
HEALHSHHTQKSLTQSPGK

### Sequences of Feline Fc Regions:

The Fc region of a feline IgG antibody comprises or consists of amino acids 231 to 447 (according to EU numbering) of the feline IgG antibody.

The amino acid sequence of the Fc domain of feline IgG1a is provided below:

(SEQ ID NO: 19)
PPEMLGGPSIFIFPPKPKDTLSISRTPEVTCLVVDLGPDDSDVQI
TWFVDNTQVYTAKTSPREEQFNSTYRVVSVLPILHQDWLKGKEFK
CKVNSKSLPSPIERTISKAKGQPHEPQVYVLPPAQEELSRNKVSV
TCLIKSFHPPDIAVEWEITGQPEPENNYRTTPPQLDSDGTYFVYS
KLSVDRSHWQRGNTYTCSVSHEALHSHHTQKSLTQSPGK

The amino acid sequence of the Fc domain of feline IG1 b is provided below:

(SEQ ID NO: 20)
PPEMLGGPSIFIFPPKPKDTLSISRTPEVTCLVVDLGPDDSDVQI
TWFVDNTQVYTAKTSPREEQFNSTYRVVSVLPILHQDWLKGKEFK
CKVNSKSLPSPIERTISKDKGQPHEPQVYVLPPAQEELSRNKVSV
TCLIEGFYPSDIAVEWEITGQPEPENNYRTTPPQLDSDGTYFLYS
RLSVDRSRWQRGNTYTCSVSHEALHSHHTQKSLTQSPGK

The amino acid sequence of the Fc domain of feline IgG2 is provided below:

(SEQ ID NO: 21)
VPEIPGAPSVFIFPPKPKDTLSISRTPEVTCLVVDLGPDDSNVQI
TWFVDNTEMHTAKTRPREEQFNSTYRVVSVLPILHQDWLKGKEFK
CKVNSKSLPSAMERTISKAKGQPHEPQVYVLPPTQEELSENKVSV
TCLIKGFHPPDIAVEWEITGQPEPENNYQTTPPQLDSDGTYFLYS
RLSVDRSHWQRGNTYTCSVSHEALHSHHTQKSLTQSPGK

Other Substitutions that Can be Included in the Bispecific Binding Agents The development of a therapeutic polypeptide or protein (e.g., a bispecific binding agent such as a bispecific antibody or Fc construct) is a complex process that entails coordination of a complex set of activities to generate the desired polypeptide or protein. These include optimization of the specificity, affinity, functional activity, expression level in engineered cell lines, long-term stability, elimination or enhancement of effector functions and development of commercially viable manufacturing and purification methods. This disclosure encompasses substitutions at one or more additional amino acid positions of the canine or feline Fc region variant that facilitates any one or more of the above goals.

In some embodiments, the canine Fc region variant described herein comprises amino acid substitutions at one or more additional amino acid positions that increase or decrease effector function and/or improve product heterogeneity.

In some embodiments, the substitutions are introduced to reduce effector function of the canine Fc region. Such substitutions may be at one or more (e.g., 1, 2, 3, 4, 5, 6, or 7) of the following positions of the canine IgG (numbering according to EU numbering): 238, 265, 297, 298, 299, 327, and 329. The substitution(s) can be to any of the other 19 amino acids. In some embodiments, the substitution is conservative. In certain non-limiting embodiments, the substituted amino acid at position 238 is Ala; the substituted amino acid at position 265 is Ala; the substituted amino acid at position 297 is Ala or Gln; the substituted amino acid at position 298 is Pro; the substituted amino acid at position 299 is Ala; the substituted amino acid at position 327 is Gly; and the substituted amino acid at position 329 is Ala. In some embodiments, the variant Fc region is from a canine IgGB or IgGC antibody. In some embodiments, the variant Fc region is from a canine IgGB antibody.

In some embodiments, substitutions are introduced to a wild type canine IgG Fc region to enhance binding to Protein A so as to facilitate purification by protein A chromatography. Such substitutions may be at one or both (e.g., 1, 2, 3, 4, 5, 6, or 7) of the following positions of the canine IgG (numbering according to EU numbering): 252 and 254. The substitution(s) can be to any of the other 19 amino acids. In some embodiments, the substitution is conservative. In certain non-limiting embodiments, the substituted amino acid at position 252 is Met; and the substituted amino acid at position 254 is Ser.

In some embodiments, the feline Fc region variant described herein comprises amino acid substitutions at one or more additional amino acid positions that increase or decrease effector function and/or improve product heterogeneity.

In some embodiments, the substitutions are introduced to reduce effector function of the feline Fc region. Such substitutions will be familiar to persons skilled in the art and may be at one or more (e.g., 1, 2, 3, 4, 5, 6, or 7) positions of the feline IgG. Illustrative examples of such substitutions include those disclosed in WO 2019/035010 A1. In some embodiments, the substitution is conservative. In some embodiments, the substitution may be at amino acid position 297 of the feline IgG (numbering according to EU numbering). In some embodiments, the substituted amino acid at position 297 is Ala or Gln.

In some embodiments, substitutions are introduced to a wild type feline IgG Fc region to enhance binding to Protein A so as to facilitate purification by protein A chromatography. Such substitutions may be at one or more (e.g., 1, 2, 3, 4, 5, 6, or 7) positions of the feline IgG. Illustrative examples of such substitutions include those disclosed in WO 2019/035010 A1.

In some embodiments, the substitutions are made to alter binding affinity of canine or feline Fc region variants described herein to FcRn as compared to a wildtype canine or feline Fc (e.g., to increase or reduce binding affinity with FcRn). In some variations, the modification can be one, two, three, or four modifications that are selected from the group consisting of: 308F, 428L, 434M and 434S, where the numbering is according to the EU numbering. In some embodiments, the Fc variant includes one or more modifications selected from the group consisting of: 252Y/428L, 428L/434H, 428L/434F, 428L/434Y, 428L/434A, 428L/434M, and 428L/434S, where the numbering is according to the EU numbering. In some embodiments, the Fc variant includes one or more modification selected from the group consisting of: 428L/434S, 308F/428L/434S, where the numbering is according to the EU numbering. In some embodiments, the Fc variant includes one or more modifications selected from the group consisting of: 2591/434S, 308F/434S, 308F/428L/434S, 2591/308F/434S, 307Q/308F/434S, 2501/308F/434S, and 308F/319L/434S, where the numbering is according to the EU numbering. A detailed description of these modifications is described in e.g., U.S. Pat. No. 8,883,973B2, which is incorporated herein by reference in its entirety.

In some embodiments, the bispecific binding agent comprises a hinge region of a canine or feline antibody. In some embodiments, modifications can be made to the hinge region of the canine or feline antibody to increase half-life. In some embodiments, the modification is 228P according to EU numbering.

In some embodiments, the binding with FcRn is pH-dependent. H310 and H435 (EU numbering) can be critical for pH-dependent binding. Thus, in some embodiments, the amino acids at position 310 (EU numbering) is histidine. In some embodiments, the amino acids at position 435 (EU numbering) is histidine. In some embodiments, the amino acids at both positions are histidine.

In some embodiments, the Fc region has MALA mutations (M234A and L235A mutations in EU numbering), or MALA-PG mutations (M234A, L235A, P329G mutations in EU numbering). In some embodiments, the Fc region has a P234A, M234A, S234A, or 1234A mutation. In some embodiments, the amino acid residue at position 234 (EU numbering) is Ala. In some embodiments, the amino acid residue at position 234 (EU numbering) is Ala. In some embodiments, the amino acid residues at positions 234 and 235 (EU numbering) are Ala.

### Pharmaceutical Compositions

In one aspect, the invention features a pharmaceutical composition comprising (i) any one of the bispecific antibodies disclosed herein or any one of the Fc constructs disclosed herein, and (ii) a pharmaceutically acceptable carrier.

To prepare pharmaceutical or sterile compositions of a bispecific antibody or Fc construct described herein, the bispecific antibody or Fc construct can be admixed with a pharmaceutically acceptable carrier or excipient. (See, e.g., Remington's Pharmaceutical Sciences and U.S. Pharmacopeia: National Formulary, Mack Publishing Company, Easton, Pa. (1984)).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). In one embodiment, the bispecific antibody or Fc construct of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, a bispecific antibody or Fc construct exhibiting high therapeutic indices are desirable. The data obtained from these cell culture assays and animal studies can be used in formulating a range of dosage for use in canines or felines. The dosage of such compounds lies preferably within a range of circulating concentrations that include the ED₅₀ with little or no toxicity. The dosage may vary within this range depending upon the dosage form employed and the route of administration.

Any suitable mode of administration can be used. Exemplary suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the bispecific antibody or Fc construct can be administered by an invasive route such as by injection. In further embodiments, the bispecific antibody or Fc construct is administered intravenously, subcutaneously, intramuscularly, intraarterially, intratumorally, or by inhalation, aerosol delivery.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer the bispecific antibody or Fc construct in a local rather than systemic manner, for example, via injection of the antibody directly into an arthritic joint or pathogen-induced lesion characterized by immunopathology, often in a depot or sustained release formulation. Furthermore, one may administer the bispecific antibody or Fc construct in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including, without limitation, the age, weight, and physical condition of the canine or feline being treated, the serum or tissue turnover rate of the therapeutic antibody, the level of symptoms, the immunogenicity of the therapeutic bispecific antibody or Fc construct, and the accessibility of the target cells in the biological matrix. Preferably, the administration regimen delivers sufficient therapeutic bispecific antibody or Fc construct to effect improvement in the target disease state, while simultaneously minimizing undesired side effects. Accordingly, the amount of biologic delivered depends in part on the particular therapeutic bispecific antibody or Fc construct and the severity of the condition being treated. Guidance in selecting appropriate doses of therapeutic antibodies is available (see, e.g., Wawrzynczak Antibody Therapy, Bios Scientific Pub. Ltd, Oxfordshire, U K (1996); Milgrom et al. New Engl. J. Med. 341:1966-1973 (1999); Slamon et al. New Engl. J. Med. 344: 783-792 (2001); Beniaminovitz et al. New Engl. J. Med. 342: 613-619 (2000); Ghosh et al. New Engl. J. Med. 348: 24-32 (2003); Lipsky et al. New Engl. J. Med. 343: 1594-1602 (2000)).

Determination of the appropriate dose of the bispecific antibody or Fc construct is made by one skilled in the art, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

### Nucleic Acids, Vectors, Host Cells, and Methods of Production

The disclosure also encompasses nucleic acid or nucleic acids encoding the bispecific binding agent (e.g., bispecific antibody or Fc construct) described herein, a vector or vectors comprising the nucleic acid or nucleic acids, and host cells comprising the nucleic acid or nucleic acids or the vector or vectors.

In one aspect, the invention features a nucleic acid or nucleic acids encoding any one of the bispecific antibodies disclosed herein or any one of the Fc constructs disclosed herein.

In another aspect, the invention features an expression vector or expression vectors comprising a nucleic acid or nucleic acids encoding any one of the bispecific antibodies disclosed herein or any one of the Fc constructs disclosed herein.

In another aspect, the invention features a host cell comprising a nucleic acid or nucleic acids encoding any one of the bispecific antibodies disclosed herein or any one of the Fc constructs disclosed herein, or an expression vector or expression vectors comprising a nucleic acid or nucleic acids encoding any one of the bispecific antibodies disclosed herein or any one of the Fc constructs disclosed herein.

In another aspect, the invention features a method of making a bispecific antibody or an Fc construct, the method comprising:
(a) providing a nucleic acid or nucleic acids of encoding any one of the bispecific antibodies disclosed herein or any one of the Fc constructs disclosed herein;
(b) expressing the nucleic acid or nucleic acids in a host cell culture, thereby producing the bispecific antibody or the Fc construct; and, optionally,
(c) collecting the bispecific antibody or the Fc construct produced in (ii) from the host cell culture.

In some embodiments, the host cell culture comprises (i) one population of host cells expressing both the first companion animal Fc region variant and the second companion animal Fc region variant or (ii) two populations of host cells comprising a first population expressing the first companion animal Fc region variant and a second population expressing the second companion animal Fc region variant.

The bispecific antibody or Fc construct described herein may be produced in bacterial or eukaryotic cells. Some polypeptides constituents of the bispecific antibody or Fc construct, e.g., Fabs, can be produced in bacterial cells, e.g., E. coli cells. Polypeptides can also be produced in eukaryotic cells such as transformed cell lines (e.g., CHO, 293E, COS, 293T, Hela). In addition, polypeptides (e.g., scFvs) can be expressed in a yeast cell such as Pichia (see, e.g., Powers et al., J Immunol Methods. 251: 123-35 (2001)), Hanseula, or Saccharomyces. To produce the bispecific antibody or Fc construct of interest, a polynucleotide or polynucleotides encoding the bispecific antibody or Fc construct is/are constructed, introduced into an expression vector or expression vectors, and then expressed in suitable host cells. To improve expression, the nucleotide sequences of the genes can be recoded without changing (or minimally changing—e.g., removal of a C-terminal residue of the heavy or light chain) the amino acid sequence. The areas for potential recoding include those associated with translation initiation, codon usage, and possible unintended mRNA splicing. Polynucleotides encoding an Fc region variant described herein would be readily envisioned by the ordinarily skilled artisan.

Standard molecular biology techniques can be used to prepare the recombinant expression vector(s), transfect the host cells, select for transformants, culture the host cells, and recover the polypeptide (e.g., bispecific antibody or Fc construct).

If the bispecific binding agent (e.g., bispecific antibody or Fc construct) is to be expressed in bacterial cells (e.g., E. coli), the expression vector may have characteristics that permit amplification of the vector in the bacterial cells. Additionally, when E. coli such as JM109, DH5α, HB101, or XL1-Blue is used as a host, the vector may have a promoter, for example, a lacZ promoter (Ward et al., 341: 544-546 (1989), araB promoter (Better et al., Science, 240: 1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the peIB signal sequence (Lei et al., J. Bacteriol., 169: 4379 (1987)) may be used as the signal sequence for antibody secretion. For bacterial expression, calcium chloride methods or electroporation methods may be used to introduce the expression vector into the bacterial cell.

If the bispecific binding agent (e.g., bispecific antibody or Fc construct) is to be expressed in animal cells such as CHO, COS, and NIH3T3 cells, the expression vector may include a promoter for expression in these cells, for example, an SV40 promoter (Mulligan et al., Nature, 277: 108 (1979)) (e.g., early simian virus 40 promoter), MMLV-LTR promoter, EF1α promoter (Mizushima et al., Nucleic Acids Res., 18: 5322 (1990)), or CMV promoter (e.g., human cytomegalovirus immediate early promoter). In addition to the nucleic acid sequence encoding the Fc region variant, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665, and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In some embodiments, the bispecific antibody or Fc construct are produced in mammalian cells. Exemplary mammalian host cells for expressing polypeptide or polypeptides (e.g., bispecific antibody or Fc construct) include Chinese Hamster Ovary (CHO cells) (including dhfr-CHO cells, described in Urlaub and Chasin (1980), Proc. Natl. Acad. Sci. USA, 77: 4216-4220, used with a DHFR selectable marker, e.g., as described in Kaufman and Sharp (1982), Mol. Biol. 159: 601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of the antibody is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

### Methods of Treatment

The bispecific binding agent (e.g., bispecific antibody or Fc construct) disclosed herein can be used to treat or prevent any disease or disorder in a companion animal (e.g., a dog or a cat) in need thereof.

In one aspect, the invention features a method of treating or preventing a disease or disorder in a companion animal in need thereof, the method comprising administering an effective amount of a composition comprising any one of the bispecific antibodies disclosed herein, any one of the Fc constructs disclosed herein, or a pharmaceutical composition comprising same to the companion animal.

In one aspect, the invention features a method of treating or preventing a canine disease or disorder in a dog in need thereof, the method comprising administering an effective amount of a composition comprising any one of the bispecific antibodies comprising canine Fc region variants disclosed herein, any one of the Fc constructs comprising canine Fc region variants disclosed herein, or a pharmaceutical composition comprising same to the dog.

In some embodiments, the canine disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, arthritis, anemia, or obesity.

In another aspect, the invention features any one of the bispecific antibodies comprising canine Fc region variants disclosed herein, any one of the Fc constructs comprising canine Fc region variants disclosed herein, or a pharmaceutical composition comprising same for use in treatment or prevention of a canine disease or disorder in a dog in need thereof.

In some embodiments, the canine disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the canine disease or disorder is atopic dermatitis, allergic dermatitis, osteoarthritic pain, arthritis, anemia, or obesity.

In another aspect, the invention features a method of treating or preventing a feline disease or disorder in a cat in need thereof, the method comprising administering an effective amount of a composition comprising any one of the bispecific antibodies comprising feline Fc region variants disclosed herein, any one of the Fc constructs comprising feline Fc region variants disclosed herein, or a pharmaceutical composition comprising same to the cat.

In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the feline disease or disorder is atopic dermatitis, allergic dermatitis, osteoarthritic pain, arthritis, anemia, or obesity.

In another aspect, the invention features any one of the bispecific antibodies comprising feline Fc region variants disclosed herein, any one of the Fc constructs comprising feline Fc region variants disclosed herein, or a pharmaceutical composition comprising same for use in treatment or prevention of a feline disease or disorder in a cat in need thereof.

In some embodiments, the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer. In some embodiments, the feline disease or disorder is atopic dermatitis, allergic dermatitis, osteoarthritic pain, arthritis, anemia, or obesity.

Any suitable companion animal (e.g., canine or feline) disease or disorder may be treated. In some embodiments, the canine or feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

In other embodiments, the canine or feline disease or disorder is atopic dermatitis, allergic dermatitis, osteoarthritic pain, arthritis, anemia, or obesity.

In some embodiments, the disease, disorder, condition, or symptoms being treated or prevented is an allergic disease, a chronic pain, an acute pain, an inflammatory disease, an autoimmune disease, an endocrine disease, a gastrointestinal disease, a skeletal/musculoskeletal disease, a cardiovascular disease, a neurological disease, a renal disease, a metabolic disease, an immunological disease, a genetic/inherited disease, a fertility related disorder, an infectious disease or a cancer. In certain embodiments, the disease or disorder being treated or prevented is atopic dermatitis, allergic dermatitis, food allergy, osteoarthritic pain, perioperative pain, dental pain, cancer pain, arthritis, anemia, obesity, or diabetes.

Antibodies may not only be used to treat or prevent disease but also to modulate normal biological function, for example, to manage fertility or behavior.

In some embodiments, the bispecific antibody or Fc construct disclosed herein, or the pharmaceutical composition comprising the bispecific antibody or Fc construct disclosed herein, is administered parenterally, by subcutaneous administration, intravenous infusion, or intramuscular injection. In some embodiments, the bispecific antibody or Fc construct disclosed herein, or the pharmaceutical composition comprising the bispecific antibody or Fc construct disclosed herein, is administered as a bolus injection or by continuous infusion over a period of time. In some embodiments, the bispecific antibody or Fc construct disclosed herein, or the pharmaceutical composition comprising the bispecific antibody or Fc construct disclosed herein, is administered by an intramuscular, an intraperitoneal, an intracerebrospinal, a subcutaneous, an intra-arterial, an intrasynovial, an intrathecal, or an inhalation route.

In some embodiments, the bispecific antibody or Fc construct disclosed herein, or the pharmaceutical composition comprising the bispecific antibody or Fc construct disclosed herein, is administered in an amount in the range of 0.01 mg/kg body weight to 50 mg/kg body weight per dose. In some embodiments, the bispecific antibody or Fc construct disclosed herein, or the pharmaceutical composition comprising the bispecific antibody or Fc construct disclosed herein, is administered 0.01 to 55 mg/kg, 0.01 to 50 mg/kg, 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, monthly, every two months, every three months, every four months, every five months, or every six months, for example. One exemplary dosage of the antibody in canines would be in the range from 0.01 mg/kg to 15 mg/kg. Thus, one or more doses of 0.01 mg/kg, 0.02 mg/kg, 0.04 mg/kg, 0.1 mg/kg, 0.2 mg/kg, 0.4 mg/kg, 1.0 mg/kg, 2.0 mg/kg, 4.0 mg/kg, 10 mg/kg, or 15 mg/kg (or any combination thereof) may be administered to the animal. One exemplary dosage of the antibody in felines would be in the range from 0.01 mg/kg to 10 mg/kg. Thus, one or more doses of 0.01 mg/kg, 0.02 mg/kg, 0.04 mg/kg, 0.1 mg/kg, 0.2 mg/kg, 0.4 mg/kg, 1.0 mg/kg, 2.0 mg/kg, 4.0 mg/kg, or 10 mg/kg (or any combination thereof) may be administered to the animal. In some embodiments, the bispecific antibody or Fc construct disclosed herein, or the pharmaceutical composition comprising the bispecific antibody or Fc construct disclosed herein, is administered 2 mg/kg body weight per dose.

In some embodiments, the bispecific binding agent (e.g., bispecific antibody or Fc construct) disclosed herein, or the pharmaceutical composition comprising the bispecific binding agent (e.g., bispecific antibody or Fc construct) disclosed herein, is administered within one, two, three, four, five, or six months, or within one, two, or three weeks, of each other. In some embodiments, the bispecific antibody or Fc construct disclosed herein), or the pharmaceutical composition comprising the bispecific antibody or Fc construct disclosed herein, is administered every week. In some embodiments, the bispecific antibody or Fc construct disclosed herein, or the pharmaceutical composition comprising the bispecific antibody or Fc construct disclosed herein, is administered every two weeks. In some embodiments, the bispecific antibody or Fc construct disclosed herein, or the pharmaceutical composition comprising the bispecific antibody or Fc construct disclosed herein, is administered every three weeks. In some embodiments, the bispecific antibody or Fc construct disclosed herein, or the pharmaceutical composition comprising the bispecific antibody or Fc construct disclosed herein, is administered every month. In some embodiments, the bispecific antibody or Fc construct disclosed herein, or the pharmaceutical composition comprising the bispecific antibody or Fc construct disclosed herein, is administered every two months. In some embodiments, the bispecific antibody or Fc construct disclosed herein, or the pharmaceutical composition comprising the bispecific antibody or Fc construct disclosed herein, is administered every three months. In some embodiments, the bispecific antibody or Fc construct disclosed herein, or the pharmaceutical composition comprising the bispecific antibody or Fc construct disclosed herein, is administered every four months. In some embodiments, the bispecific antibody or Fc construct disclosed herein, or the pharmaceutical composition comprising the bispecific antibody or Fc construct disclosed herein, is administered every five months. In some embodiments, the bispecific antibody or Fc construct disclosed herein, or the pharmaceutical composition comprising the bispecific antibody or Fc construct disclosed herein, is administered every six months. In some embodiments, the bispecific antibody or Fc construct disclosed herein, or the pharmaceutical composition comprising the bispecific antibody or Fc construct disclosed herein, is administered to a dog or a cat at one time or over a series of treatments. In some embodiments, the dose is administered once per week for at least two or three consecutive weeks, and in some embodiments, this cycle of treatment is repeated two or more times, optionally interspersed with one or more weeks of no treatment.

In some embodiments, the bispecific binding agent (e.g., bispecific antibody or Fc construct) disclosed herein, or the pharmaceutical composition comprising the bispecific antibody or Fc construct disclosed herein, is administered in combination, concurrently, sequentially, or in conjunction with one or more further therapeutic agents. In some embodiments, the bispecific antibody or Fc construct disclosed herein, or the pharmaceutical composition comprising the bispecific antibody or Fc construct disclosed herein, is administered in combination, with one or more further therapeutic agents. In some embodiments, the bispecific antibody or Fc construct disclosed herein, or the pharmaceutical composition comprising the bispecific antibody or Fc construct disclosed herein, is administered concurrently with one or more further therapeutic agents. In some embodiments, the bispecific antibody or Fc construct disclosed herein, or the pharmaceutical composition comprising the bispecific antibody or Fc construct disclosed herein, is administered sequentially with one or more further therapeutic agents. In some embodiments, the bispecific antibody or Fc construct disclosed herein, or the pharmaceutical composition comprising the bispecific antibody or Fc construct disclosed herein, is administered in conjunction with one or more further therapeutic agents. Any suitable further therapeutic agents may be used.

### Diagnosis

The bispecific binding agent (e.g., bispecific antibody or Fc construct) disclosed herein can also be used for various diagnostic purposes, for example, to determine whether a dog or a cat has any particular disease or disorder. In some embodiments, the bispecific antibody or Fc construct may comprise a binding domain. The binding domain can specifically bind to a protein, subunit, domain, motif, and/or epitope as described herein (e.g., a maker for cancer cells). In some embodiments the bispecific antibody or Fc construct further comprises a labeling group. In general, label groups fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g., horseradish peroxidase, s-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antibody via spacer arms of various lengths to reduce potential steric hindrance. Various methods for labelling proteins are known in the art and may be used in performing the present invention.

In some embodiments, the labeling group is a probe, a dye (e.g., a fluorescent dye), or a radioactive isotope (e.g., ³H, ¹⁴C, ²²Na, ³⁶Cl, ³⁵S, ³³P, or ¹²⁵I)

Specific labels can also include optical dyes, including, but not limited to, chromophores, phosphors and fluorophores, with the latter being specific in many embodiments. Fluorophores can be either “small molecule” fluores, or proteinaceous fluores.

The fluorescent label can be any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malachite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, which is incorporated by reference in its entirety.

Suitable proteinaceous fluorescent labels also include, but are not limited to, green fluorescent protein, including a Renilla, Ptilosarcus, or Aequorea species of GFP (Chalfie et al., 1994, Science 263: 802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H1J9; Stauber, 1998, Biotechniques 24: 462-471; Heim et al., 1996, Curr. Biol. 6: 178-182), enhanced yellow fluorescent protein (EYFP, Clontech Laboratories, Inc.), luciferase (Ichiki et al., 1993, J. Immunol. 150: 5408-5417), s galactosidase (Nolan et al., 1988, Proc. Natl. Acad. Sci. USA. 85: 2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references in this paragraph are expressly incorporated herein by reference in the entirety.

### Assays

### FcγRI and FcγRIII Binding:

Binding to FcγRI and FcγRIII is a measure of the ability of an antibody to mediate ADCC. In order to assess this property for an antibody an assay to measure binding of the antibody to FcγRI and FcγRIII can be conducted using methods known in the art.

### C1q Binding:

Binding to the first component of complement, C1q, is a measure of the ability of an antibody to mediate complement-dependent cytotoxicity (CDC). In order to assess this property for an antibody, an assay to measure binding of the antibody to C1q can be conducted using methods known in the art.

### Half-Life:

Methods of measuring half-life of an antibody are well known in the art. See, e.g., Booth et al., MAbs, 10(7): 1098-1110 (2018). As an example, the half-life of an antibody (e.g., a feline antibody) can be measured by injection of the antibody into an animal model (e.g., a dog model) and measuring levels of the antibody in the serum over a certain period of time. Exemplary animal models include non-human primate models and transgenic mouse models. The transgenic mouse models can be null for mouse FcRn alpha chain and express the canine FcRn alpha transgene (e.g., under the control of a constitutive promoter). The canine FcRn alpha chain can pair in vivo with the mouse R2-microglobulin protein forming a functional chimeric FcRn heterodimer. As an example, the half-life of a canine antibody can be measured by injection of the antibody into a dog model and measuring levels of the antibody in the serum over a certain period of time.

### EXAMPLES

### Example 1. Evaluation of Knob-In-Hole (KiH) Mutations in Canine IgGB Constant Domain

In the first attempt to evaluate whether knob-in-hole mutations can create a heterodimer for canine IgGs, canine IgGB was mutated on the “knob” side at T366W and S354C. For the “hole” side, mutations were made at T366S, L368A, Y407V, and Y349C (as described in human Fc regions in, e.g., U.S. Pat. No. 5,731,168 and Schaefer et al., 2011, Proc. Natl. Acad. Sci. USA, 108: 11187-11192). These mutations were in a constant domain that already contained half-life extension mutations (A426Y+T286L, as described in U.S. Pat. No. 11,434,276, which is incorporated by reference herein in its entirety) and the MALA mutation (M234A and L235A) to reduce effector function. For expression purposes, the Protein A binding site was knocked out of the “hole” chain by mutating H435R and Y436F to allow for preferential purification of the heterodimer (as described in U.S. Pat. No. 8,586,713, which is incorporated by reference herein in its entirety). Additionally, the C-terminal lysine was removed from the sequence (see Table 4).

TABLE 4
Sequences of canine IgGB constant
domain with knob-in-hole mutations

| Chain | Sequence |
|---|---|
| Canine IgGB | ASTTAPSVFPLAPSCGSTSGSTVALACLVS |
| Constant | GYFPEPVTVSWNSGSLTSGVHTFPSVLQSS |
| Knob | GLYSLSSMVTVPSSRWPSETFTCNVAHPAS |
| (SEQ ID NO. | KTKVDKPVPKRENGRVPRPPDCPKCPAPEA |
| 361) | AGGPSVFIFPPKPKDTLLIARTPEVTCVVV |
| | DLDPEDPEVQISWFVDGKQMQLAKTQPREE |
| | QFNGTYRVVSVLPIGHQDWLKGKQFTCKVN |
| | NKALPSPIERTISKARGQAHQPSVYVLPPC |
| | REELSKNTVSLWCLIKDFFPPDIDVEWQSN |
| | GQQEPESKYRTTPPQLDEDGSYFLYSKLSV |
| | DKSRWQRGDTFICYVMHEALHNHYTQESLS |
| | HSPG* |
| Canine IgGB | ASTTAPSVFPLAPSCGSTSGSTVALACLVS |
| Constant | GYFPEPVTVSWNSGSLTSGVHTFPSVLQSS |
| Hole | GLYSLSSMVTVPSSRWPSETFTCNVAHPAS |
| (SEQ ID NO. | KTKVDKPVPKRENGRVPRPPDCPKCPAPEA |
| 362) | AGGPSVFIFPPKPKDTLLIARTPEVTCVVV |
| | DLDPEDPEVQISWFVDGKQMQLAKTQPREE |
| | QFNGTYRVVSVLPIGHQDWLKGKQFTCKVN |
| | NKALPSPIERTISKARGQAHQPSVCVLPPS |
| | REELSKNTVSLSCAIKDFFPPDIDVEWQSN |
| | GQQEPESKYRTTPPQLDEDGSYFLVSKLSV |
| | DKSRWQRGDTFICYVMHEALHNRFTQESLS |
| | HSPG* |

The evaluation of canine knob-in-hole mutations was performed using two test target variable regions and light chains. Both antibodies were assessed with both knob and hole mutations. Bispecific antibodies (biAbs) were expressed in a 1:1:1:1 ratio in Chinese hamster ovary (CHO) cells and purified using Protein A resin. Resulting biAbs were analyzed for binding, aggregation, purity, and correct chain pairing.

Concentration was measured using A280 on a NANODROP™ OneC instrument (Thermo Fisher). Transient yields were in the normal range of in-house canine antibody expression.

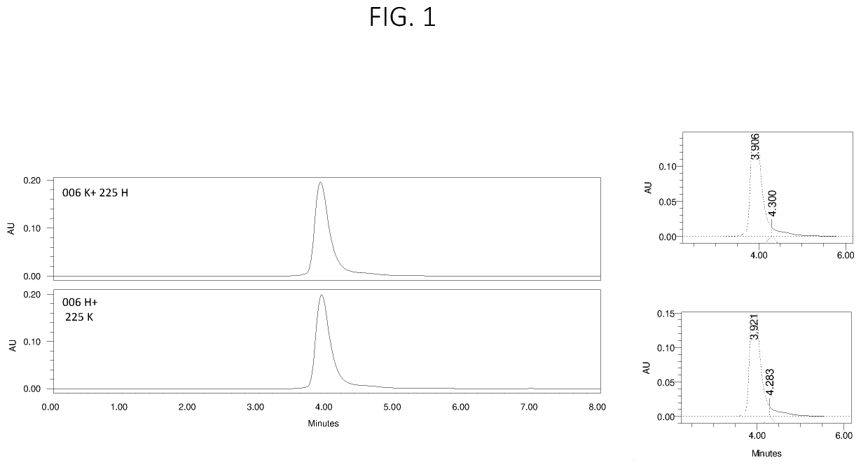

Size exclusion was performed on a Waters ALLIANCE™ e2796 Bioseparations Module using a Sepax ZENIX™ SEC-150 (7.8×200 mm, 3 μm) column to evaluate the presence and prevalence of high molecular weight species (HMWS) and low molecular weight species (LMWS). The mobile phase was 20 mM sodium phosphate, 0.3 M NaCl, pH 6.8, with an isocratic flow of 1.0 mL/min for 20 mins. Both orientations of knob-in-hole antibodies contained no HMWS and had a main peak of 92-93% purity. There was 7-8% of a LMWS which was a shoulder of the main peak as shown in the chromatograms in FIG. 1.

Chain pairing was assessed by intact mass using liquid chromatography mass spectrometry (LC-MS).

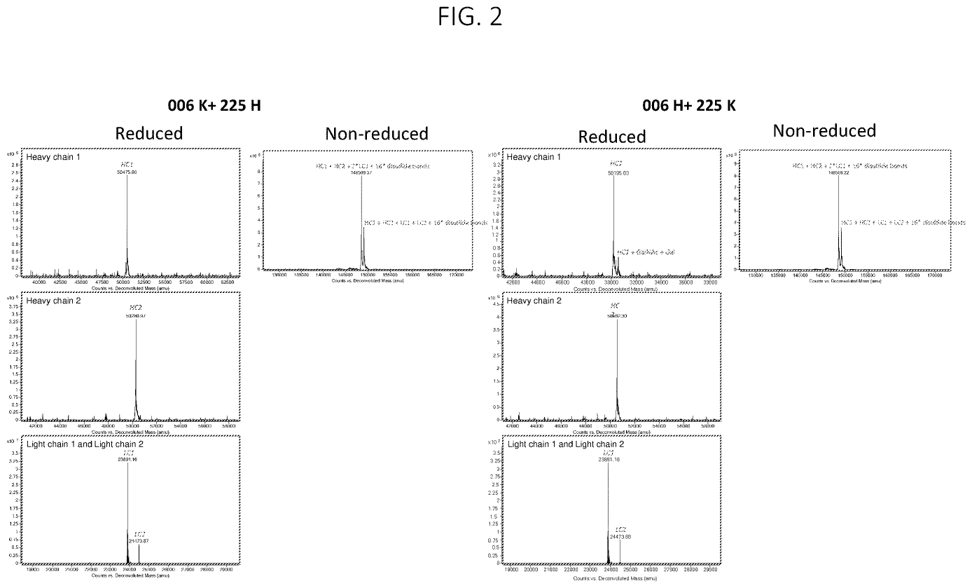

Samples were deglycosylated with PNGase F and run either reduced or non-reduced over a PLRP-S (2.1×50 mm, 8 μm) column on an Agilent 1290 Infinity II uPLC followed by an Agilent 6530 Electrospray Ionization Quadruple Time-of-Flight Mass Spectrometer (ESI-QToF). The assignments of measured masses were determined using BioConfirm 10.0 software and are summarized in Table 5, and the deconvoluted subunit mass spectra are shown in FIG. 2.

TABLE 5
Intact mass results of canine knob-in-hole constructs

| | | Theoretical | Observed | |
|---|---|---|---|---|
| | Name | Mass (Da) | Mass (Da) | Assignments |
| Reduced | 006 | 50473.91 | 50475.60 | HC1 (006 Knob) |
| | Knob + | (Assuming 1 | | |
| | 225 | Asn to Asp) | | |
| | Hole | 50281.99 | 50280.97 | HC2 (225 Hole) |
| | | (Assuming 1 | | |
| | | Asn to Asp) | | |
| | | 23890.72 | 23891.16 | LC1 (006) |
| | | 24473.35 | 24473.87 | LC2 (225) |
| Non- | 006 | 149087.71 | 148509.37 | HC1 + HC2 + 2*LC1 + |
| Reduced | Knob + | | 149091.09 | 16*disulfide bonds |
| | 225 | | | HC1 + HC2 + LC1 + |
| | Hole | | | LC2 + 16*disulfide |
| | | | | bonds |
| Reduced | 225 | 50195.60 | 50195.03 | HC1 (006 Hole) |
| | Knob + | (Assuming 1 | 50559.58 | HC1 + GalNAc + Gal |
| | 006 | Asn to Asp) | | |
| | Hole | 50560.30 | 50562.30 | HC2 (225 Knob) |
| | | (Assuming 1 | | |
| | | Asn to Asp) | | |
| | | 23890.72 | 23891.16 | LC1 (006) |
| | | 24473.35 | 24473.88 | LC2 (225) |
| Non- | 225 | 149087.71 | 148508.22 | HC1 + HC2 + 2*LC1 + |
| Reduced | Knob + | | 149093.26 | 16*disulfide bonds |
| | 006 | | | HC1 + HC2 + LC1 + |
| | Hole | | | LC2 + 16*disulfide |
| | | | | bonds |

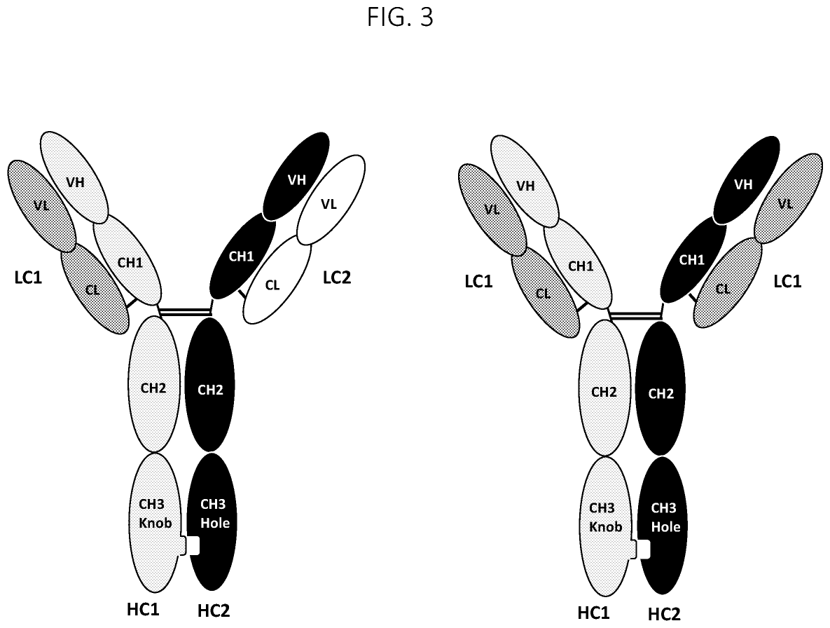

Amino acid sequence of each chain was confirmed by both non-reduced and reduced masses. Non-reduced intact mass analysis confirmed the assembly of the knob-in-hole constructs. However, the major species observed in both orientations was HCl+HC2+2*LC1+16*disulfide bonds instead of the expected HCl+HC2+LC1+LC2+16*disulfide bonds. The proportion of the deconvoluted peaks was 70% with the 2*LC1 versus 30% with LC1+LC2. As anticipated, the light chain mispairing was not prevented with the current mutations, but the knob-in-hole is present in both species (schematic of confirmed species in FIG. 3).

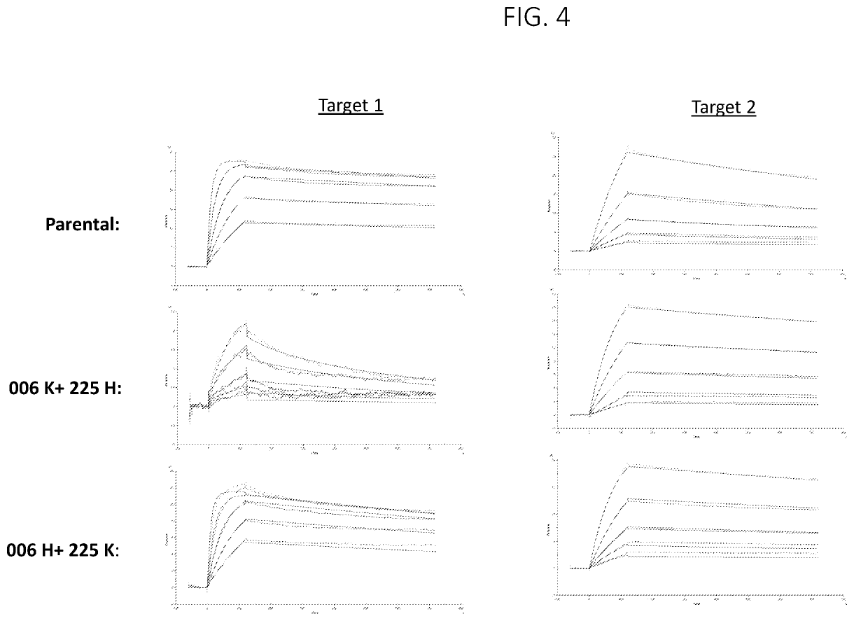

Affinity to the different targets of the bispecific was evaluated by surface plasmon resonance (SPR) on a BIACORE™ T200. Antibodies were captured using a Protein A Series S chip. Antigen binding was then assessed at multiple concentrations starting at 50 nM (Target 1) or 100 nM (Target 2) using PBSP+running buffer (Cytiva) with a flow rate of 30 μL/min. The length of the association time was 120s and the dissociation time was run for 600s. The chip surface was regenerated with 10 mM glycine. Reference-subtracted sensorgrams were fitted to a 1:1 binding model using BIACORE™ T200 Evaluation software. The data is shown in Table 6 below and the sensorgrams in FIG. 4. Affinity was retained for Target 2 in both orientations. However, for 006 Knob+225 Hole, there was noticeable loss in binding to Target 1.

TABLE 6
Affinity of knob-in-hole bispecific constructs to both antigens

| Construct | Antigen | ka (1/Ms) | kd (1/s) | KD (M) |
|---|---|---|---|---|
| 006 | Target 1 | 1.52E+06 | 2.05E−04 | 1.35E−10 |
| 006 knob/225 hole | Target 1 | 6.48E+05 | 2.91E−03 | 6.36E−09 |
| 225 knob/006 hole | Target 1 | 1.78E+06 | 4.66E−04 | 2.64E−10 |
| 225 | Target 2 | 5.97E+04 | 5.30E−04 | 8.88E−09 |
| 006 knob/225 hole | Target 2 | 1.19E+05 | 4.58E−04 | 3.95E−09 |
| 225 knob/006 hole | Target 2 | 8.39E+04 | 3.53E−04 | 6.91E−09 |

### Example 2. Evaluation of VHH-Fc with Knob-In-Hole Mutations in Canine IgGB Constant Domain

To continue the knob-in-hole evaluation with canine IgGs, VHH constructs were tested in place of the variable region and light chain. This could create a bispecific antibody that does not have a light chain mispairing concern. To generate the VHHs, llamas were immunized with either Target 2 or Target 3, peripheral blood mononuclear cells (PBMCs) from llamas with high titers against the immunizing antigen were isolated and the RNA was isolated. cDNA was generated from the RNA and phage display libraries with VHH domains were generated. VHH phage display libraries underwent selections and screens with either canine Target 2 or Target 3. ELISA-positive VHHs were sequenced and unique VHHs were synthesized with an 8× His tag at the C-terminus. A subset of VHH were expressed in E. coli and purified with nickel chromatography. Purified VHHs were then screened for affinity and top binders were used in the following examples. Target 2 VHH 02F09R3 was formatted with the full hinge (PKRENGRVPRPPDCPKCP, SEQ ID NO. 363; or VPKRENGRVPRPPDCPKCP, SEQ ID NO. 364) followed by the CH2 and CH3 of canine IgGB (Sequences in Table 7). In one case, the construct contained a linker between the VHH and hinge (GPGGQ, SEQ ID NO. 38) and in another without the addition of the linker. These both were expressed with no knob-in-hole mutations for evaluation of VHH-Fc as a standalone molecule.

TABLE 7
Sequences of VHH canine Fc

| Construct | Sequence |
|---|---|
| 02F09R3-Fc | QVQLVESGGGLVQAGGSLRLSCVASGSTFS |
| (with linker) | NLVMAWYRQAPGKQREWVARISLAGNTIYA |
| (SEQ ID NO. | DSVKGRFTISRDNAKNTVYLQMNSLKIEDT |
| 365) | AVYYCSRGGVDWGQGTQVTVSSGPGGQVPK |
| | RENGRVPRPPDCPKCPAPEAAGGPSVFIFP |
| | PKPKDTLLIARTPEVTCVVVDLDPEDPEVQ |
| | ISWFVDGKQMQTAKTQPREEQFNGTYRVVS |
| | VLPIGHQDWLKGKQFTCKVNNKALPSPIER |
| | TISKARGQAHQPSVYVLPPSREELSKNTVS |
| | LTCLIKDFFPPDIDVEWQSNGQQEPESKYR |
| | TTPPQLDEDGSYFLYSKLSVDKSRWQRGDT |
| | FICYVMHEALHNHYTQESLSHSP* |
| 02F09R3-Fc | QVQLVESGGGLVQAGGSLRLSCVASGSTFS |
| (no linker) | NLVMAWYRQAPGKQREWVARISLAGNTIYA |
| (SEQ ID NO. | DSVKGRFTISRDNAKNTVYLQMNSLKIEDT |
| 366) | AVYYCSRGGVDWGQGTQVTVSSVPKRENGR |
| | VPRPPDCPKCPAPEAAGGPSVFIFPPKPKD |
| | TLLIARTPEVTCVVVDLDPEDPEVQISWFV |
| | DGKQMQTAKTQPREEQFNGTYRVVSVLPIG |
| | HQDWLKGKQFTCKVNNKALPSPIERTISKA |
| | RGQAHQPSVYVLPPSREELSKNTVSLTCLI |
| | KDFFPPDIDVEWQSNGQQEPESKYRTTPPQ |
| | LDEDGSYFLYSKLSVDKSRWQRGDTFICYV |
| | MHEALHNHYTQESLSHSP* |
| 02F09R3-Knob | QVQLVESGGGLVQAGGSLRLSCVASGSTFS |
| (SEQ ID NO. | NLVMAWYRQAPGKQREWVARISLAGNTIYA |
| 367) | DSVKGRFTISRDNAKNTVYLQMNSLKIEDT |
| | AVYYCSRGGVDWGQGTQVTVSSGPGGQVPK |
| | RENGRVPRPPDCPKCPAPEAAGGPSVFIFP |
| | PKPKDTLLIARTPEVTCVVVDLDPEDPEVQ |
| | ISWFVDGKQMQLAKTQPREEQFNGTYRVVS |
| | VLPIGHQDWLKGKQFTCKVNNKALPSPIER |
| | TISKARGQAHQPSVYVLPPCREELSKNTVS |
| | LWCLIKDFFPPDIDVEWQSNGQQEPESKYR |
| | TTPPQLDEDGSYFLYSKLSVDKSRWQRGDT |
| | FICYVMHEALHNHYTQESLSHSPG* |
| 01E03R3-Hole | SVQLVESGGGLAQPGGSLRLSCVASGSTFS |
| (SEQ ID NO. | DNAAGWYRQAPEKQRELVAAISIHDRTIYS |
| 368) | DSVRGRFTISRDNVKNTVSLQMDNLGPEDT |
| | AVYYCNVGGTYYTFGAYAPGDYWGQGTLVT |
| | VSSGPGGQVPKRENGRVPRPPDCPKCPAPE |
| | AAGGPSVFIFPPKPKDTLLIARTPEVTCVV |
| | VDLDPEDPEVQISWFVDGKQMQLAKTQPRE |
| | EQFNGTYRVVSVLPIGHQDWLKGKQFTCKV |
| | NNKALPSPIERTISKARGQAHQPSVCVLPP |
| | SREELSKNTVSLSCAIKDFFPPDIDVEWQS |
| | NGQQEPESKYRTTPPQLDEDGSYFLVSKLS |
| | VDKSRWQRGDTFICYVMHEALHNRFTQESL |
| | SHSPG* |

In a separate construct of VHHs 02F09R3-Fc, the constant canine IgGB sequence was mutated on the “knob” side at T366W and S354C. For the “hole” side mutations, Target 3 VHH 01E03R3 was selected and mutated at T366S, L368A, Y407V, and Y349C. Mutations were in a constant domain that already contained half-life extension mutations (A426Y+T286L) and the MALA mutation (M234A and L235A) to reduce effector function. For expression purposes, the Protein A binding site was knocked out of the “hole” chain by mutating H435R and Y436F to allow for preferential purification of the heterodimer. Additionally, the C-terminal lysine was removed from the sequences.

VHH-Fcs were expressed in CHO cells and purified using Protein A resin. Resulting VHH-Fcs were analyzed for binding, aggregation, purity, and correct chain pairing.

Concentration was measured using A280 on a NANODROP™ OneC instrument (Thermo Fisher). Transient yields of the VHH-Fc (with or without the linker) were higher than that observed with the VHH-knob-in-hole constructs; the latter were in the normal range of in-house canine antibody expression.

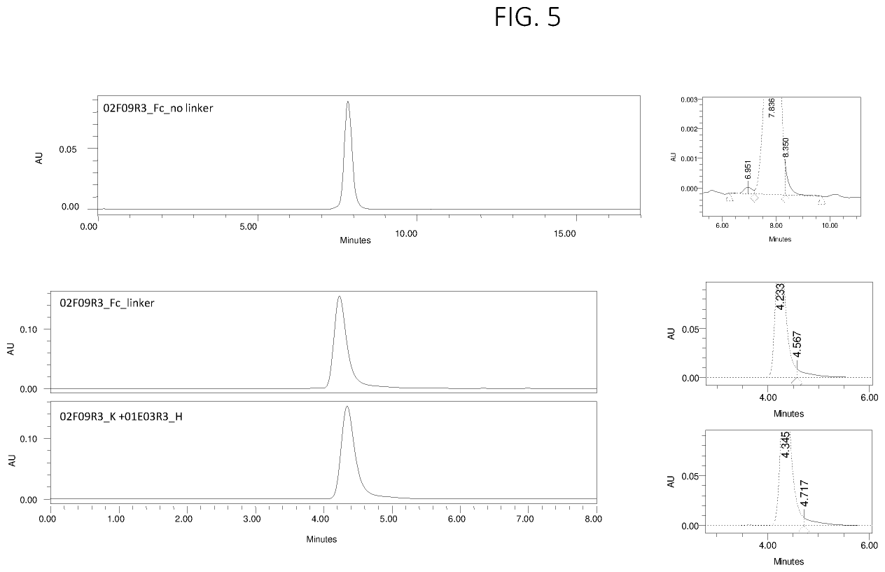

Size exclusion was performed on a Waters ALLIANCE™ e2796 using YMC-Pack-Diol-200 (300×8 mm I.D. S-5 μm, 20 nm) or a Sepax ZENIX™ SEC-150 (7.8×200 mm, 3 μm) column to evaluate the presence and prevalence of high molecular weight species (HMWS) and low molecular weight species (LMWS). The mobile phase was 20 mM sodium phosphate, 0.3 M NaCl, pH 6.8, with an isocratic flow of 1.0 mL/min for 20 min. Retention times for columns were noticeably different. Both orientations of knob-in-hole antibodies contained no HMWS and had a main peak of 92-93% purity. There was 7-8% of a LMWS which was a shoulder of the main peak as shown in the chromatograms in FIG. 1. One VHH-Fc constructs contained minor HMWS (0.2%) while all constructs had a main peak of 92-99% purity. There was 0.6-7.2% of a LMWS which was a shoulder of the main peak as shown in the chromatograms in FIG. 5.

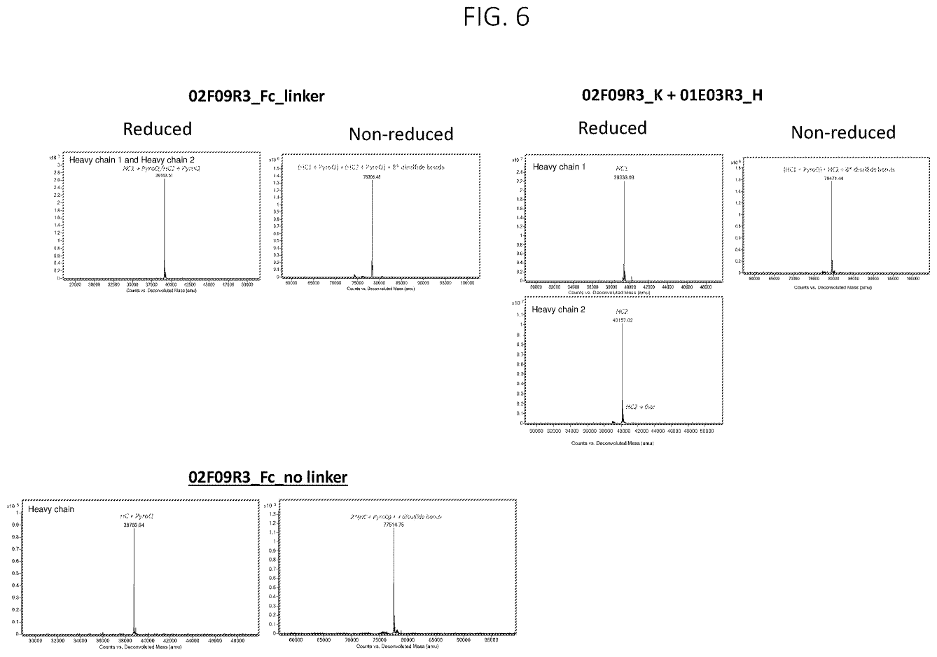

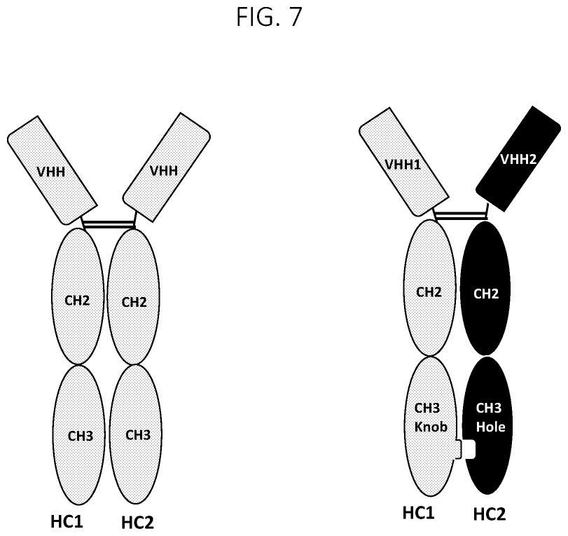

Chain pairing was assessed by intact mass using liquid chromatography mass spectrometry as described above. The assignments of measured masses were determined using BioConfirm 10.0 software and are summarized in Table 8, and the deconvoluted subunit mass spectra are shown in FIG. 6. Amino acid sequence of each chain was confirmed by both non-reduced and reduced masses. Non-reduced intact mass analysis confirmed the assembly of the VHH-Fc and the VHH knob-in-hole constructs (schematic of confirmed species in FIG. 7).

TABLE 8
Intact mass results of VHH-Fc and VHH knob-in-hole constructs

| | | Theoretical | Observed | |
|---|---|---|---|---|
| | Name | Mass (Da) | Mass (Da) | Assignments |
| Reduced | 02F09R3_Fc | 39178.20 | 39163.51 | HC1 + PyroQ*2 |
| | | (Assuming 1 | | |
| | | Asn to Asp) | | |
| Non- | 02F09R3_Fc | 78356.40 | 78308.48 | (HC1 + PyroQ) + (HC1 + |
| Reduced | | | | PyroQ) + 8*disulfide bonds |
| Reduced | 02F09R3_Fc_no | 38781.80 | 38766.64 | HC + PyroQ |
| | linker | (Assuming 1 | | |
| | | Asn to Asp) | | |
| Non- | 02F09R3_Fc_no | 77563.60 | 77514.75 | 2*(HC + PyroQ) + 4 (?) |
| Reduced | linker | (Assuming 1 | | disulfide bonds |
| | | Asn to Asp) | | |
| Reduced | 02F09R3_Fc_K | 39348.48 | 39333.93 | HC1 + PyroQ |
| | 01E03R3_Fc_H | (Assuming 1 | | (02F09R3_Fc_K) |
| | | Asn to Asp) | | |
| | | 40154.06 | 40157.02 | HC2 (01E03R3_Fc_H) |
| | | | 40319.37 | HC2 + Gal |
| Non- | 02F09R3_Fc_K | 79502.54 | 79471.44 | (HC1 + PyroQ) + HC2 + |
| Reduced | 01E03R3_Fc_H | | | 8*disulfide bonds |

### Example 3. Evaluation of mAb/VHH-Fc-Knob-In-Hole

This format of monovalent bispecific antibodies consists of one side as a half antibody and the other as a VHH-Fc. One side contains the knob mutations while the other maintains the hole mutations, thus combining Example 1 and Example 2 constructs (Sequences in Table 4 and Table 7). Light chain mispairing is not an issue since pairing can only occur on the half antibody side. Two orientations were assessed with: (1) 006 knob and 01E03R3-Hole; and (2) 02F09R3-Knob and 006 hole.

Bispecific mAbNHHs were expressed in a 1:1:1 ratio (HC1:HC2:LC1) in CHO cells and purified using Protein A resin. Resulting biAbs were analyzed for binding, aggregation, purity, and correct chain pairing.

Concentration was measured using A280 on a NANODROP™ OneC instrument (Thermo Fisher). Transient yields were in the normal range of in-house canine antibody expression.

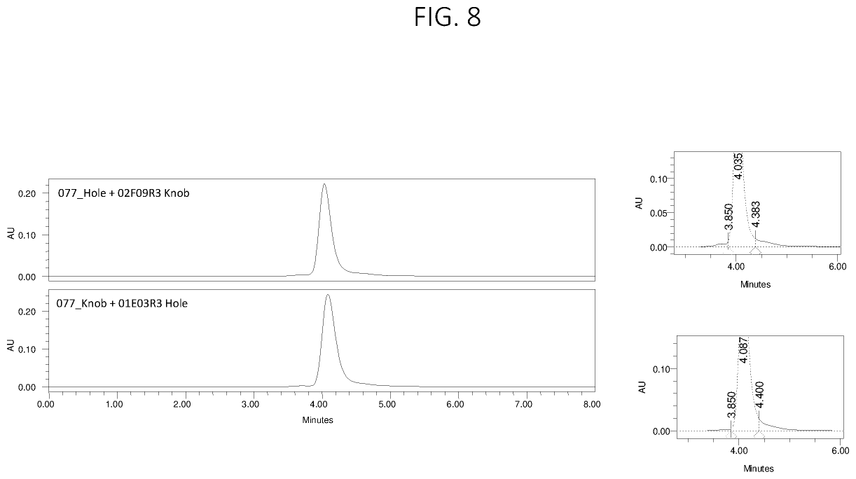

Size exclusion was performed as described above using the Sepax ZENIX™ SEC-150. Both orientations of knob-in-hole antibodies contained minor HMWS (1-2.5%) and had a main peak of 89-91% purity. There was 7-8% of a LMWS which was a shoulder of the main peak as shown in the chromatograms in FIG. 8. Retention time was longer than a normal IgG but shorter than the VHH-Fc constructs as expected with the molecular weight being between the two.

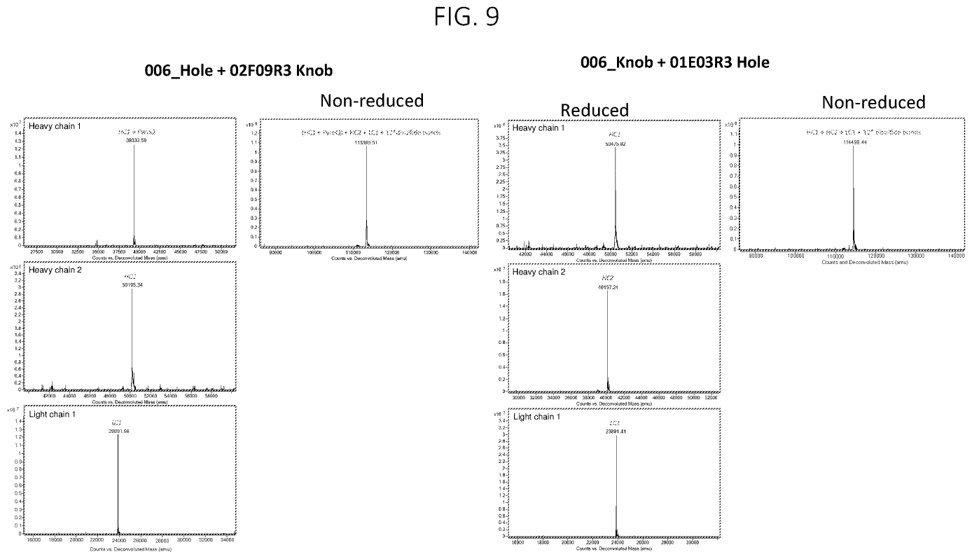

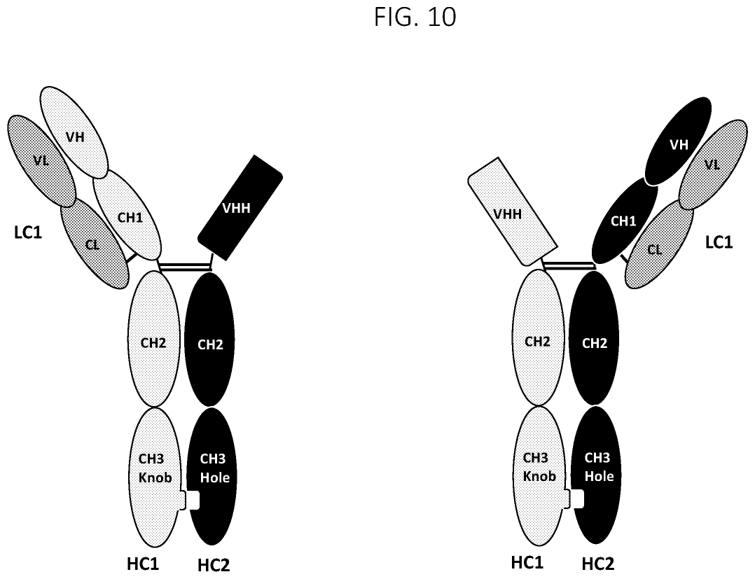

Chain pairing was assessed by intact mass using liquid chromatography mass spectrometry as described above. The assignments of measured masses were determined using BioConfirm 10.0 software and are summarized in Table 9, and the deconvoluted subunit mass spectra are shown in FIG. 9. Amino acid sequence of each chain was confirmed by both non-reduced and reduced masses. Non-reduced intact mass analysis confirmed the assembly of the knob-in-hole constructs (schematic of confirmed species in FIG. 10).

TABLE 9
Intact mass results of IgG/VHH knob-in-hole constructs

| | | Theoretical | Observed | |
|---|---|---|---|---|
| | Name | Mass (Da) | Mass (Da) | Assignments |
| Reduced | 006 knob + | 50473.91 | 50475.82 | HC1 (006 Knob) |
| | 01E03R3-Hole | (Assuming 1 | | |
| | | Asn to Asp) | | |
| | | 40155.07 | 40157.24 | HC2 (01E03R3-Hole) |
| | | (Assuming 1 | | |
| | | Asn to Asp) | | |
| | | 23890.72 | 23891.41 | LC1 (006) |
| Non- | 006 knob + | 114495.50 | 114498.44 | HC1 + HC2 + LC1 + |
| reduced | 01E03R3-Hole | | | 12*disulfide bonds |
| Reduced | 02F09R3-Knob | 39348.48 | 39333.59 | HC1 + PyroQ |
| | and 006 hole | (Assuming 1 | | (02F09R3-Knob) |
| | | Asn to Asp) | | |
| | | 50195.60 | 50195.34 | HC2 (006 Hole) |
| | | (Assuming 1 | | |
| | | Asn to Asp) | | |
| | | 23890.72 | 23891.66 | LC1 (006) |
| Non- | 02F09R3-Knob | 113410.60 | 113395.51 | (HC1 + PyroQ) + HC2 + LC1 + |
| reduced | and 006 hole | | | 12*disulfide bonds |

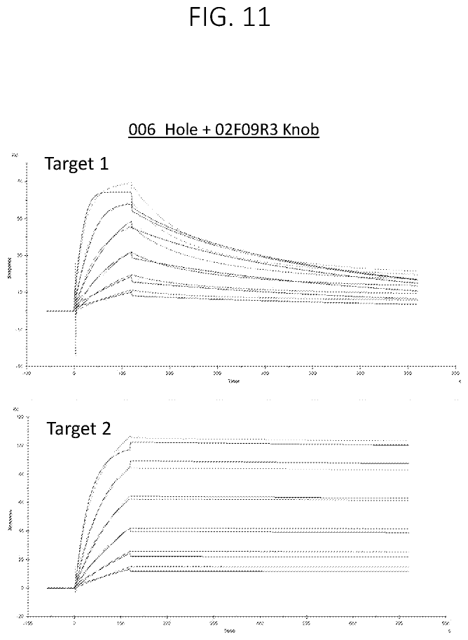

Affinity to the Target 1 and Target 2 of the bispecific was evaluated by SPR on a BIACORE™ T200. Antibodies were captured using an anti-canine coupled CM5 chip. Antigen binding was then assessed as described above. Reference-subtracted sensorgrams were fitted to a 1:1 binding model using BIACORE™ T200 Evaluation software. The data is shown in Table 10 below and the sensorgrams in FIG. 11. Affinity was retained for both targets.

TABLE 10
Affinity of VHH-Fc and VHH knob-in-hole bispecific constructs

| Construct | Antigen | ka (1/Ms) | kd (1/s) | KD (M) |
|---|---|---|---|---|
| 006 | Target 1 | 1.22E+06 | 2.55E−03 | 2.08E−09 |
| 02F09R3-Knob and | Target 1 | 1.06E+06 | 3.29E−03 | 3.09E−09 |
| 006 Hole | | | | |
| 02F09R3-Fc | Target 2 | 2.93E+05 | 6.24E−05 | 2.13E−10 |
| 02F09R3-Knob and | Target 2 | 3.19E+05 | 3.71E−05 | 1.16E−10 |
| 006 hole | | | | |

### Example 4. Evaluation of Full Monoclonal Canine IgGB Antibody with a C-Terminal Linker Followed by VHH

This format of a bivalent bispecific antibody consists of monoclonal antibodies with a linker at the C-terminus followed by VHH. No knob-in-in hole sequence was necessary since there is only one heavy chain construct. The construct only contains one light chain, for Target 1, so mispairing is not an issue. The sequence of this construct is in Table 11.

TABLE 11
Sequences of mAb-linker-VHH

| Construct | Sequence |
|---|---|
| 006_GGS_02F09 | -variable region- |
| R3 | ASTTAPSVFPLAPSCGSTSGSTVALACLVS |
| Heavy Chain | GYFPEPVTVSWNSGSLTSGVHTFPSVLQSS |
| (SEQ ID NO. | GLYSLSSMVTVPSSRWPSETFTCNVAHPAS |
| 369) | KTKVDKPVPKRENGRVPRPPDCPKCPAPEA |
| | AGGPSVFIFPPKPKDTLLIARTPEVTCVVV |
| | DLDPEDPEVQISWFVDGKQMQTAKTQPREE |
| | QFNGTYRVVSVLPIGHQDWLKGKQFTCKVN |
| | NKALPSPIERTISKARGQAHQPSVYVLPPS |
| | REELSKNTVSLTCLIKDFFPPDIDVEWQSN |
| | GQQEPESKYRTTPPQLDEDGSYFLYSKLSV |
| | DKSRWQRGDTFICYVMHEALHNHYTQESLS |
| | HSPGGSGGSQVQLVESGGGLVQAGGSLRLS |
| | CVASGSTFSNLVMAWYRQAPGKQREWVARI |
| | SLAGNTIYADSVKGRFTISRDNAKNTVYLQ |
| | MNSLKIEDTAVYYCSRGGVDWGQGTQVTVS |
| | SGPGGQ* |

The IgG/VHH construct was expressed in a 1:1 ratio (HC:LC) in CHO cells and purified using Protein A resin. Resulting biAb was analyzed for binding, aggregation, purity, and correct chain pairing.

Concentration was measured using A280 on a NANODROP™ OneC instrument (Thermo Fisher). Transient yields were in the normal range of in-house canine antibody expression.

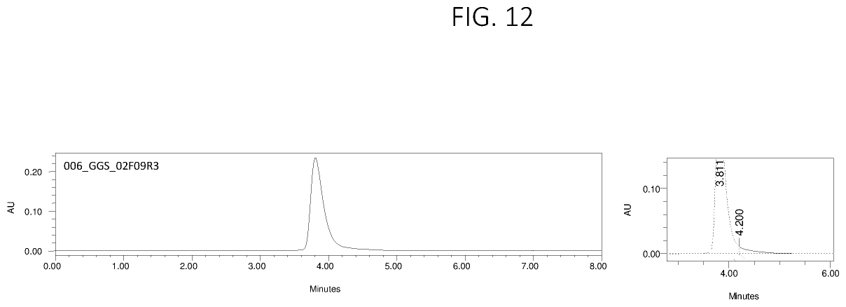

Size exclusion was performed as described above using the Sepax ZENIX™ SEC-150. Both orientations of knob-in-hole antibodies contained no HMWS and had a main peak of 94% purity. There was 6% of a LMWS which was a shoulder of the main peak as shown in the chromatograms in FIG. 12. Retention time was shorter than a normal IgG as expected since this construct is larger in size.

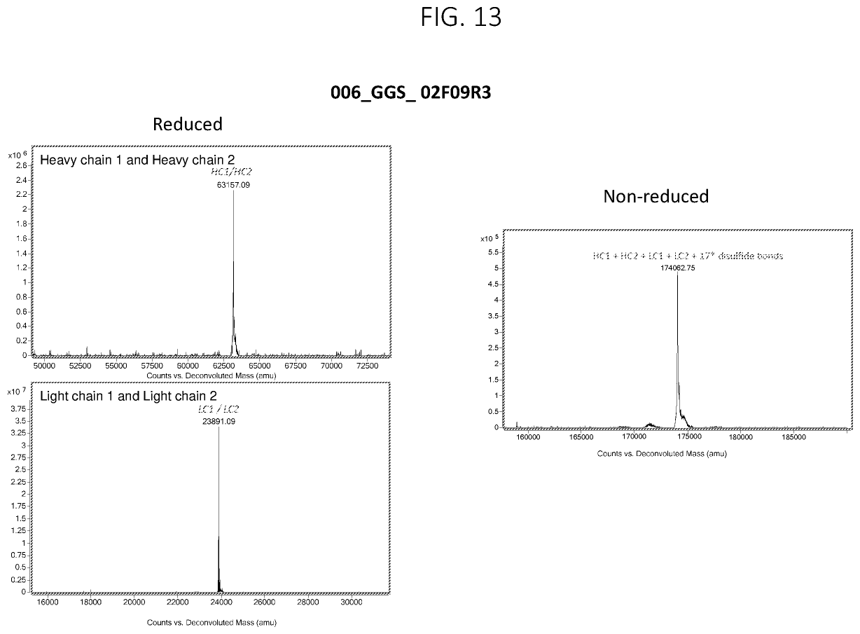

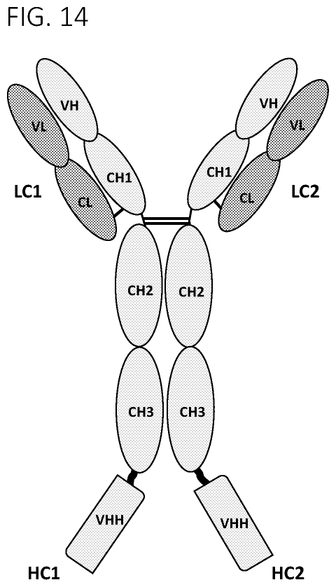

Chain pairing was assessed by intact mass using liquid chromatography mass spectrometry as described above. Assignments of measured masses were determined using BioConfirm 10.0 software and are summarized in Table 12, and the deconvoluted subunit mass spectra are shown in FIG. 13. Amino acid sequence of each chain was confirmed by both non-reduced and reduced masses. Non-reduced intact mass analysis confirmed the correct assembly of the constructs (schematic of confirmed species in FIG. 14).

TABLE 12
Intact mass results of IgG-linker-VHH constructs

| | | Theoretical | Observed | |
|---|---|---|---|---|
| | Name | Mass (Da) | Mass (Da) | Assignments |
| Reduced | 006_GGS_{—} | 63154.97 | 63157.09 | HC1 |
| | 02F09R3 | (Assuming 1 | | |
| | | Asn to Asp) | | |
| | | 23890.72 | 23891.09 | LC1 |
| Non- | 006_GGS_{—} | 174057.10 | 174062.75 | HC1*2 + L1*2 + |
| reduced | 02F09R3 | | | 17*disulfide bonds |

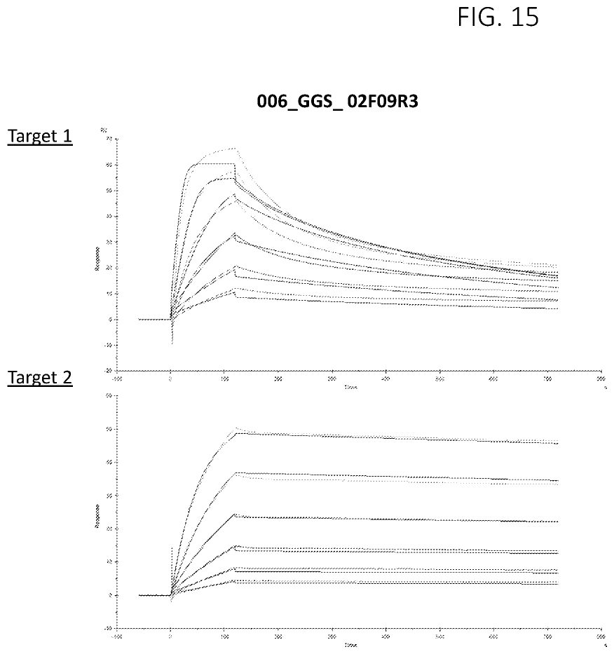

Affinity to the different targets of the bispecific was evaluated by SPR on a BIACORE™ T200. Antibodies were captured using an anti-canine coupled CM5 chip and antigen binding was assessed as described above. Reference-subtracted sensorgrams were fitted to a 1:1 binding model using BIACORE™ T200 Evaluation software. The data is shown in Table 13 below and the sensorgrams in FIG. 15. Affinity was retained for both targets.

TABLE 13
Affinity of VHH-Fc and VHH knob-in-hole bispecific

| Construct | Antigen | ka (1/Ms) | kd (1/s) | KD (M) |
|---|---|---|---|---|
| 006_GGS_02F09R3 | Target 1 | 1.73E+06 | 4.19E−03 | 2.42E−09 |
| 006_GGS_02F09R3 | Target 2 | 1.90E+05 | 1.07E−04 | 5.60E−10 |

### Example 5. Evaluation of Knob-In-Hole Mutations in Feline IgG1a Constant Domain

To evaluate whether knob-in-hole mutations can create a heterodimer for feline IgGs, feline IgG1a was mutated on the “knob” side at T366W. For the “hole” side, mutations were made at T366S, L368A, and Y407V (as described for human Fc regions in U.S. Pat. No. 5,731,168, which is incorporated herein by reference in its entirety). Cysteine mutations were not added for some sequences since A354 was non-conserved compared to the human counterpart S354 (Y349 is conserved in the feline sequence). However, in other entities, these were tested to determine if non-conserved A354 could be mutated to cysteine.

These mutations were in a constant domain that already contained half-life extension mutations (T286E+Q311V+S428Y; as described in U.S. Patent Application Publication No. US 2022/0259282, which is incorporated herein by reference in its entirety) and the MALA mutation (M234A and L235A) to reduce effector function. For expression purposes, the Protein A binding site was knocked-out of the “hole” chain by mutating H435R and Y436F to allow for preferential purification of the heterodimer (as described in U.S. Pat. No. 8,586,713, which is incorporated herein by reference in its entirety). Additionally, the C-terminal lysine was removed from the sequence (see Table 14).

TABLE 14
Sequences of feline knob-into-hole constructs

| Construct | Sequence |
|---|---|
| Constant | ASTTAPSVFPLAPSCGTTSGATVALACLVLGYFPE |
| Feline IgG1a | PVTVSWNSGALTSGVHTFPAVLQASGLYSLSSMVT |
| Knob | VPSSRWLSDTFTCNVAHPPSNTKVDKTVRKTDHPP |
| (SEQ ID NO. | GPKPCDCPKCPPPEAAGGPSIFIFPPKPKDTLSIS |
| 370) | RTPEVTCLVVDLGPDDSDVQITWFVDNTQVYEAKT |
| | SPREEQFNSTYRVVSVLPILHVDWLKGKEFKCKVN |
| | SKSLPSPIERTISKAKGQPHEPQVYVLPPAQEELS |
| | RNKVSVWCLIKSFHPPDIAVEWEITGQPEPENNYR |
| | TTPPQLDSDGTYFVYSKLSVDRSHWQRGNTYTCSV |
| | YHEALHSHHTQKSLTQSPG** |
| Constant | ASTTAPSVFPLAPSCGTTSGATVALACLVLGYFPE |
| Feline IgG1a | PVTVSWNSGALTSGVHTFPAVLQASGLYSLSSMVT |
| Hole | VPSSRWLSDTFTCNVAHPPSNTKVDKTVRKTDHPP |
| (SEQ ID NO. | GPKPCDCPKCPPPEAAGGPSIFIFPPKPKDTLSIS |
| 371) | RTPEVTCLVVDLGPDDSDVQITWFVDNTQVYEAKT |
| | SPREEQFNSTYRVVSVLPILHVDWLKGKEFKCKVN |
| | SKSLPSPIERTISKAKGQPHEPQVYVLPPAQEELS |
| | RNKVSVSCAIKSFHPPDIAVEWEITGQPEPENNYR |
| | TTPPQLDSDGTYFVVSKLSVDRSHWQRGNTYTCSV |
| | YHEALHSRFTQKSLTQSPG** |
| Constant | ASTTAPSVFPLAPSCGTTSGATVALACLVLGYFPE |
| Feline IgG1a | PVTVSWNSGALTSGVHTFPAVLQASGLYSLSSMVT |
| Knob+A354C | VPSSRWLSDTFTCNVAHPPSNTKVDKTVRKTDHPP |
| (SEQ ID NO. | GPKPCDCPKCPPPEAAGGPSIFIFPPKPKDTLSIS |
| 372) | RTPEVTCLVVDLGPDDSDVQITWFVDNTQVYEAKT |
| | SPREEQFNSTYRVVSVLPILHVDWLKGKEFKCKVN |
| | SKSLPSPIERTISKAKGQPHEPQVYVLPPCQEELS |
| | RNKVSVWCLIKSFHPPDIAVEWEITGQPEPENNYR |
| | TTPPQLDSDGTYFVYSKLSVDRSHWQRGNTYTCSV |
| | YHEALHSHHTQKSLTQSPG** |
| Constant | ASTTAPSVFPLAPSCGTTSGATVALACLVLGYFPE |
| Feline IgG1a | PVTVSWNSGALTSGVHTFPAVLQASGLYSLSSMVT |
| Hole+Y349C | VPSSRWLSDTFTCNVAHPPSNTKVDKTVRKTDHPP |
| (SEQ ID NO. | GPKPCDCPKCPPPEAAGGPSIFIFPPKPKDTLSIS |
| 373) | RTPEVTCLVVDLGPDDSDVQITWFVDNTQVYEAKT |
| | SPREEQFNSTYRVVSVLPILHVDWLKGKEFKCKVN |
| | SKSLPSPIERTISKAKGQPHEPQVCVLPPAQEELS |
| | RNKVSVSCAIKSFHPPDIAVEWEITGQPEPENNYR |
| | TTPPQLDSDGTYFVVSKLSVDRSHWQRGNTYTCSV |
| | YHEALHSRFTQKSLTQSPG** |

The evaluation of feline IgGs with knob-into-hole mutations was performed using two test targets variable regions and light chains. Both antibodies were assessed with both knob and hole mutations. Bispecific antibodies were expressed in a 1:1:1:1 ratio in CHO cells and purified using Protein A resin. Resulting biAbs were analyzed for binding, aggregation, purity, and correct chain pairing.

Concentration was measured using A280 on a NANODROP™ OneC instrument (Thermo Fisher). Transient yields were in the normal range of in-house feline antibody expression.

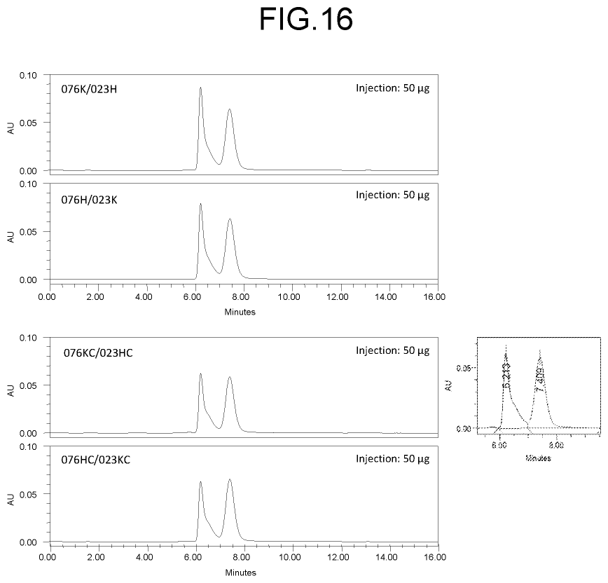

Size exclusion was performed on a Waters Alliance e2796 using YMC-Pack-Diol-200 (300×8 mm I.D., S-5 μm, 20 nm) column to evaluate the presence and prevalence of high molecular weight species (HMWS) and low molecular weight species (LMWS). The mobile phase was 20 mM sodium phosphate, 0.3 M NaCl, pH 6.8, with an isocratic flow of 1.0 mL/min for 18 mins. All constructs contained two major species, one with a retention time at 6.21 mins ranging from 42-48% of peak area and the other, comparable to monoclonal mAbs, at 7.4 mins ranging from 50-57% of the peak area. There was no LMWS observed in the chromatograms as shown in FIG. 16.

Chain pairing was assessed by intact mass using liquid chromatography mass spectrometry. Samples were either non-deglycosylated or deglycosylated with PNGase F and run either reduced or non-reduced over a PLRP-S (2.1×50 mm, 8 μm) column on an Agilent 1290 Infinity II uPLC followed by an Agilent 6530 ESI-QToF. The assignments of measured masses were determined using BioConfirm 10.0 software and are summarized in Table 15.

TABLE 15
Intact mass results of feline knob-in-hole constructs

| | | Theoretical | Observed | |
|---|---|---|---|---|
| Condition | Name | Mass (Da) | Mass (Da) | Assignments |
| Non-deglycosylated | 076 Knob + | 50349.7 | 51795.4* | HC1 (076 Knob) + G0F |
| and Reduced | 023 Hole | 49271.4 | 50488.4 | HC2 (023 Hole) + Man5 |
| | | | 50513.9 | HC2 (023 Hole) + G0F − |
| | | | | GlcNAc |
| | | | 50716.7* | HC2 (023 Hole) + G0F |
| | | 23840.5 | 23841.2* | LC1 (076) |
| | | 24077.8 | 24078.3 | LC2 (023) |
| Deglycosylated and | 076 Knob + | 147268.02 | 147271.7* | 2 × LC1 + HC1 + HC2 |
| Non-reduced | 023 Hole | | 147511.6 | 2 × LC1 + HC1 + HC2 + |
| | | | | 2 × Cysteinylation |
| | | 148583.55 | 148573.02 | 2 × HC1 + LC1 + LC2 |
| Non-deglycosylated | 023 Knob + | 50173.5 | 51618.4* | HC1 (076 Hole) + G0F |
| and Reduced | 076 Hole | 49447.55 | 50689.5 | HC2 (023 Knob) + G0F − |
| | | | | GlcNAc |
| | | | 50892.8* | HC2 (023 Knob) + G0F |
| | | 23840.5 | 23841.2* | LC1 (076) |
| | | 24077.8 | 24078.2 | LC2 (023) |
| Deglycosylated and | 023 Knob + | 147268.0 | 147272.0* | 2 × LC2 + HC1 + HC2 |
| Non-reduced | 076 Hole | | | |
| Non-deglycosylated | 076_Knob_A354C + | 49271.4 | 50716.3* | HC1 (076 Knob_A354C) + |
| and Reduced | 023 Hole_Y349C | | | G0F |
| | | 50381.7 | 51826.7* | HC2 (023 Hole_Y349C) + |
| | | | | G0F |
| | | 23840.5 | 23841.3* | LC1 (076) |
| | | 24077.8 | 24078.6 | LC2 (023) |
| Deglycosylated and | 076_Knob_A354C + | 147300.08 | 147419.4 | 2 × LC1 + HC1 + HC2 + |
| Non-reduced | 023 Hole_Y349C | | | Cysteinylation |
| | | | 147660.3* | 2 × LC1 + HC1 + HC2 + |
| | | | | 4 × Cysteinylation |
| Non-deglycosylated | 023_Knob_A354C + | 50173.5 | 51617.5* | HC1 (076 Hole_Y349C) + |
| and Reduced | 076 Hole_Y349C | | | G0F |
| | | 49479.6 | 50927.2* | HC2 (023 Knob_A354C) + |
| | | | | G0F |
| | | 23840.5 | 23841.2* | LC1 (076) |
| | | 24077.8 | 24078.6 | LC2 (023) |
| Deglycosylated and | 023_Knob_A354C + | 147300.1 | 147669.6 | 2 × LC2 + HC1 + HC2 + |
| Non-reduced | 076 Hole_Y349C | | | 4 × Cysteinylation |

*Major species

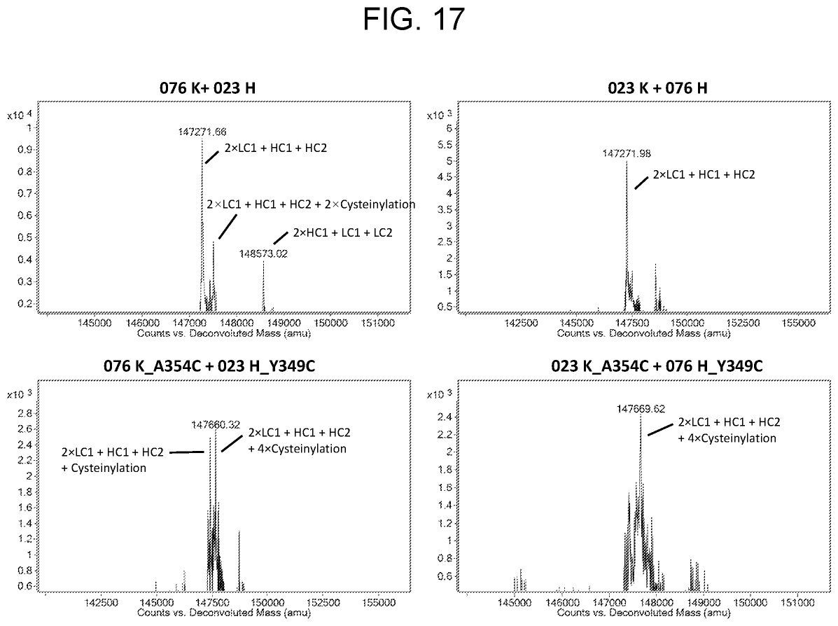

The amino acid sequence of each chain was confirmed by reduced intact mass analysis. However, only one light chain was seen to be a major species. The proportion of the deconvoluted peaks was higher for LC1 (~74-92%) compared to LC2 (~8-26%). Non-reduced intact mass analysis confirmed the assembly of the knob-in-hole constructs. However, there was knob-knob species observed with the 076 Knob+023 Hole construct. No hole-hole construct could be purified due to the Protein A knockout on that chain. Additionally, the major species observed in constructs without the additional cysteines was “HCl+HC2+2×LC1.” With the additional cysteines, the major construct was “HCl+HC2+2×LC1+4×Cysteinylation” (FIG. 17). The presence of this cysteinylation suggests that the disulfide bond was not forming properly and that the strategy of introducing the cysteine to increase stability into the non-conserved A354 location was not effective as expected. As anticipated, the light chain mispairing was not prevented with the current mutations, but the knob-in-hole is the major species in all constructs.

Finally, the affinity of the constructs to different targets of the bispecific antibody was evaluated by surface plasmon resonance (SPR) on a BIACORE™ T200. Antibodies were captured using an anti-feline coupled CM5 chip. Antigen binding was then assessed at multiple concentrations starting at 50 nM using PBSP+running buffer (Cytiva) with a flow rate of 30 μL/min. The length of the association time was 120 s and the dissociation time was run for 600 s. The chip surface was regenerated with 10 mM glycine.

Reference-subtracted sensorgrams were fitted to a 1:1 binding model using BIACORE™ T200 Evaluation software. The data is shown in Table 16 below. Affinity was retained for Target 1 in both orientations. However, there was a noticeable loss in binding to Target 2 for all constructs. This can be explained by the lack of the Target 2 light chain in the constructs as observed in the intact mass data. The knob-in-hole mutations that were made did not completely prevent light chain mispairing.

TABLE 16
Affinity of feline bispecific antibodies to target antigens

| Construct | Antigen | ka (1/Ms) | kd (1/s) | KD (M) | Rmax (RU) |
|---|---|---|---|---|---|
| 076 | Target 1 | 4.57E+05 | 3.05E−07 | <1e−12 | 29.9 |
| 076-Knob and 023-Hole | Target 1 | 4.72E+05 | 2.02E−09 | <1e−12 | 15.8 |
| 076-Hole and 023-Knob | Target 1 | 4.45E+05 | 2.73E−05 | 6.13E−11 | 14.2 |
| 076-Knob_A354C and | Target 1 | 4.53E+05 | 5.12E−08 | <1e−12 | 14.8 |
| 023-Hole_Y349C | | | | | |
| 076-Hole_Y349C and | Target 1 | 4.67E+05 | 3.64E−07 | <1e−12 | 15.4 |
| 023-Knob_A354C | | | | | |
| 023 | Target 2 | 3.65E+06 | 1.29E−07 | <1e−12 | 32.7 |
| 076-Knob and 023-Hole | Target 2 | No binding | | | |
| 076-Hole and 023-Knob | Target 2 | No binding | | | |
| 076-Knob_A354C and | Target 2 | 5.71E+06 | 2.09E−07 | <1e−12 | 6.7 |
| 023-Hole_Y349C | | | | | |
| 076-Hole_Y349C and | Target 2 | 6.02E+06 | 9.11E−08 | <1e−12 | 8.9 |
| 023-Knob_A354C | | | | | |

1. A bispecific antibody comprising:
(a) a first binding domain that binds to a first antigen, wherein the first binding domain is linked to a first companion animal Fc region variant; and
(b) a second binding domain that binds to a second antigen, wherein the second binding domain is linked to a second companion animal Fc region variant;
wherein at least one of the first binding domain and the second binding domain comprises a single-domain antibody.

2. The bispecific antibody of claim 1, wherein the first binding domain and the second binding domain each specifically binds to an antigen independently selected from the group consisting of NGF, TrKA, ADAMTS, IL-1, IL-2, IL-4, IL-4R, Angiotensin type 1 (AT1) receptor, Angiotensin type 2 (AT2) receptor, IL-5, IL-12, IL-13, IL-31, IL-31R, IL-33, CD3, CD20, CD47, CD52, and complement system complex.

3. The bispecific antibody of claim 1 or 2, wherein the first binding domain and/or the second binding domain comprises an antibody, an antibody fragment, or a ligand-binding portion of a receptor.

4. The bispecific antibody of claim 3, wherein the antibody fragment is selected from the group consisting of Fab, single chain variable fragment (scFv), Fv, Fab′, Fab′-SH, F(ab′)₂, and diabody.

5. The bispecific antibody of any one of claims 1-4, wherein the single-domain antibody is linked to the first companion animal Fc region variant or the second companion animal Fc region variant either directly or via a peptide linker.

6. The bispecific antibody of any one of claims 1-5, wherein the single-domain antibody is a VHH domain.

7. The bispecific antibody of claim 6, wherein:
(i) the VHH domain comprises a C-terminal residue and the first companion animal Fc region variant or the second companion animal Fc region variant comprises an N-terminal residue, and the C-terminal residue of the VHH domain is linked either directly or via a peptide linker to the N-terminal residue of the first companion animal Fc region variant or the second companion animal Fc region variant; or
(ii) the VHH domain comprises an N-terminal residue, and the first companion animal Fc region variant or the second companion animal Fc region variant comprises a C-terminal residue, and the N-terminal residue of the VHH domain is linked either directly or via a peptide linker to the C-terminal residue of the first companion animal Fc region variant or the second companion animal Fc region variant.

8. The bispecific antibody of any one of claims 5-7, wherein the peptide linker comprises an amino acid sequence selected from the group consisting of:
(a)
(SEQ ID NO: 38)
GPGGQ;
(b)
(SEQ ID NO: 363)
PKRENGRVPRPPDCPKCP;
(c)
(SEQ ID NO: 364)
VPKRENGRVPRPPDCPKCP;
(d)
(SEQ ID NO: 22)
FNECRCTDTPPCPVPEP;
(e)
(SEQ ID NO: 23)
PKRENGRVPRPPDCPKCPAPEM;
(f)
(SEQ ID NO: 24)
AKECECKCNCNNCPCPGCGL;
(g)
(SEQ ID NO: 25)
PKESTCKCISPCPVPES;
(h)
(SEQ ID NO: 26)
PKESTCKCIPPCPVPES;
(i)
(SEQ ID NO: 27)
KTDHPPGPKPCDCPKCP;
and
(j)
(SEQ ID NO: 29)
KTASTIESKTGEGPKCP.

9. The bispecific antibody of any one of claims 1-8, wherein the first companion animal Fc region variant and the second companion animal Fc region variant are canine Fc region variants.

10. The bispecific antibody of claim 9, wherein the first canine Fc region variant or the second canine Fc region variant comprises an amino acid sequence that is at least 95% identical to an amino acid sequence selected from the group consisting of SEQ ID NOs: 9 to 12.

11. The bispecific antibody of claim 9 or 10, wherein the first canine Fc region variant and the second canine Fc region variant comprise complementary dimerization selectivity modules that promote dimerization between the first canine Fc region variant and the second canine Fc region variant.

12. The bispecific antibody of claim 11, wherein the first canine Fc region variant and the second canine Fc region variant each comprises a protuberance or a cavity, wherein if the first canine Fc region comprises a protuberance, the second canine Fc region comprises a cavity, and wherein if the first canine Fc region comprises a cavity, the second canine Fc region comprises a protuberance.

13. The bispecific antibody of claim 11 or 12, wherein the first canine Fc region variant and the second canine Fc region variant comprise amino acid substitutions selected from the group consisting of:
(a) S354C and T366W in the first canine Fc region variant; and Y349C, T366S, L368A, and Y407V in the second canine Fc region variant;
(b) T366W in the first canine Fc region variant; and T366S, L368A, and Y407V in the second canine Fc region variant;
(c) R392D and K409D in the first canine Fc region variant; and E356K and D399K in the second canine Fc region variant;
(d) S364H and F405A in the first canine Fc region variant; and Y349T and T394F in the second canine Fc region variant;
(e) F405L in the first canine Fc region variant; and K409R in the second canine Fc region variant;
(f) T366L, R392L, and T394W in the first canine Fc region variant; and L351Y, F405A, and Y407V in the second canine Fc region variant;
(g) K360E and K409W in the first canine Fc region variant; and S347R, D399V, and F405T in the second canine Fc region variant;
(h) Y349C, K360E, and K409W in the first canine Fc region variant; and S347R, S354C, D399V, and F405T in the second canine Fc region variant;
(i) K370E and K409W in the first canine Fc region variant; and E357N, D399V, and F405T in the second canine Fc region variant;
(j) K360D, D399M, and Y407A in the first canine Fc region variant; and Q345R, S347R, T366V, and K409V in the second canine Fc region variant;
(k) Y349S, T366M, K370Y, and K409V in the first canine Fc region variant; and E356G, E357D, S364Q, and Y407A in the second canine Fc region variant;
(l) L351 D and L368E in the first canine Fc region variant; and L351K and T366K in the second canine Fc region variant;
(m) L368D and K370S in the first canine Fc region variant; and E356Q and S364K in the second canine Fc region variant; or
(n) T366Y in the first canine Fc region variant; and T366S, L368A, and Y407T in the second canine Fc region variant;
wherein the amino acid positions are based on EU numbering.

14. The bispecific antibody of any one of claims 9-13, wherein the first canine Fc region variant comprises a first charged region and the second canine Fc region variant comprises a second charged region, and wherein the first charged region forms a charge pair with the second charged region.

15. The bispecific antibody of claim 14, wherein the first charged region comprises a basic amino acid residue and the second charged region comprises an acidic amino acid residue.

16. The bispecific antibody of claim 15, wherein the first canine Fc region variant and the second canine Fc region variant further comprise CH1 domains comprising the following amino acid substitutions:
S183D in the first canine Fc region variant; and S183K in the second canine Fc region variant.

17. The bispecific antibody of claim 15 or 16, wherein the first canine Fc region variant and the second canine Fc region variant comprise CH3 domains comprising amino acid substitutions selected from the group consisting of:
(a) K409D in the first canine Fc region variant and D399K in the second canine Fc region variant;
(b) K390D and K409D in the first canine Fc region variant; and E356K and D399K in the second canine Fc region variant;
(c) K390D and K409D in the first canine Fc region variant; and E357K and D399K in the second canine Fc region variant; and
(d) K370D and K409D and in the first canine Fc region variant; and E357K and D399K in the second canine Fc region variant.

18. The bispecific antibody of any one of claims 15-17, wherein the first canine Fc region variant and the second canine Fc region variant further comprise CL domains comprising the following amino acid substitutions:
S176K in the first canine Fc region variant; and S176D in the second canine Fc region variant.

19. The bispecific antibody of any one of claims 9-18, wherein the first canine Fc region variant and/or the second canine Fc region variants further comprise at least one of the following amino acid substitutions:
(a) 252Y and, optionally, at least one amino acid substitution selected from the group consisting of 251 D or 251E; 285N or 285D; 286D; 307Q; 308P; 315D; 430A or 430K; 433K; 435Y; and 436H;
(b) 252M and, optionally, at least one amino acid substitution selected from the group consisting of 251 D or 251E; 256D or 256F; 285N or 285D; 286D; 307Q; 308P; 315D; 430A or 430K; 433K; 435Y; and 436H;
(c) 434R;
(d) 426Y and, optionally, at least one amino acid substitution selected from the group consisting of 286F, 286W, 286L, or 286Y; 312P; 434R; and 436H;
(e) 426H and, optionally, at least one amino acid substitution selected from the group consisting of 286F, 286W, 286L, or 286Y; 312P; 434R; and 436H;
(f) 426F and, optionally, at least one amino acid substitution selected from the group consisting of 286F, 286W, 286L, or 286Y; 312P; 434R; and 426H; and
(g) 434R and, optionally, at least one amino acid substitution selected from the group consisting of 286L; 286Y; 312P; and 436H;
wherein the amino acid positions are based on EU numbering.

20. The bispecific antibody of any one of claims 1-8, wherein the first companion animal Fc region variant and the second companion animal Fc region variant are feline Fc region variants.

21. The bispecific antibody of claim 20, wherein the first feline Fc region variant or the second feline Fc region variant comprises an amino acid sequence that is at least 95% identical to an amino acid sequence selected from the group consisting of SEQ ID NOs: 19 to 21.

22. The bispecific antibody of claim 20 or 21, wherein the first feline Fc region variant and the second feline Fc region variant comprise complementary dimerization selectivity modules that promote dimerization between the first feline Fc region variant and the second feline Fc region variant.

23. The bispecific antibody of claim 22, wherein the first feline Fc region variant and the second feline Fc region variant each comprises a protuberance or a cavity, wherein if the first feline Fc region comprises a protuberance, the second feline Fc region comprises a cavity, and wherein if the first feline Fc region comprises a cavity, the second feline Fc region comprises a protuberance.

24. The bispecific antibody of claim 22 or 23, wherein the first feline Fc region variant and the second feline Fc region variant comprise amino acid substitutions selected from the group consisting of:
(a) T366W in the first feline Fc region variant; and T366S, L368A, and Y407V in the second feline Fc region variant;
(b) T366W in the first feline Fc region variant; and T366S, L368A, and Y398T in the second feline Fc region variant;
(c) A354C and T366W in the first feline Fc region variant; and Y349C, T366S, L368A, and Y407V in the second feline Fc region variant;
(d) R392D and K409D in the first feline Fc region variant; and E356K and D399K in the second feline Fc region variant;
(e) S364H and F405A in the first feline Fc region variant; and Y349T and T394F in the second feline Fc region variant;
(f) F405L in the first feline Fc region variant; and K409R in the second feline Fc region variant;
(g) T366L, R392L, and T394W in the first feline Fc region variant; and L351Y, F405A, and Y407V in the second feline Fc region variant;
(h) R360E and K409W in the first feline Fc region variant; and Q347R, D399V, and F405T in the second feline Fc region variant;
(i) Y349C, R360E, and K409W in the first feline Fc region variant; and Q347R, A354C, D399V, F405T in the second feline Fc region variant;
(j) K370E and K409W in the first feline Fc region variant; and E357N, D399V, and F405T in the second feline Fc region variant;
(k) R360D, D399M, and Y407A in the first feline Fc region variant; and E345R, Q347R, T366V, and K409V in the second feline Fc region variant;
(l) Y349S, K370Y, T366M, and K409V in the first feline Fc region variant; and E356G, E357D, S364Q, and Y407A in the second feline Fc region variant;
(m) L351 D and L368E in the first feline Fc region variant; and L351K and T366K in the second feline Fc region variant;
(n) L368D and K370S in the first feline Fc region variant; and E356Q and S364K in the second feline Fc region variant; and
(o) T366Y in the first feline Fc region variant; and T366S, L368A, and Y407T in the second feline Fc region variant;
wherein the amino acid positions are based on EU numbering.

25. The bispecific antibody of any one of claims 20-24, wherein the first feline Fc region variant comprises a first charged region and the second feline Fc region variant comprises a second charged region, and wherein the first charged region forms a charge pair with the second charged region.

26. The bispecific antibody of claim 25, wherein the first charged region comprises a basic amino acid residue and the second charged region comprises an acidic amino acid residue.

27. The bispecific antibody of claim 26, wherein the first feline Fc region variant and the second feline Fc region variant further comprise CH1 domains comprising the following amino acid substitutions:
S183D in the first feline Fc region variant; and S183K in the second feline Fc region variant.

28. The bispecific antibody of claim 26 or 27, wherein the first feline Fc region variant and the second feline Fc region variant comprise CH3 domains comprising amino acid substitutions selected from the group consisting of:
(a) K409D in the first feline Fc region variant and D399K in the second feline Fc region variant; and
(b) K370D and K409D and in the first feline Fc region variant; and E357K and D399K in the second feline Fc region variant.

29. The bispecific antibody of any one of claims 26-28, wherein the first feline Fc region variant and the second feline Fc region variant further comprise CL domains comprising the following amino acid substitutions:
S176K in the first feline Fc region variant; and S176D in the second feline Fc region variant.

30. The bispecific antibody of any one of claims 20-29, wherein the first and/or second feline Fc region variants further comprise at least one of the following amino acid substitutions:
(a) at least one amino acid substitution selected from the group consisting of 286E; 311V; and 428Y;
(b) two or more amino acid substitutions selected from the group consisting of 286E; 311V; and 428Y; and
(c) 286E, 311V, and 428Y;
wherein the amino acid positions are based on EU numbering.

31. An Fc construct comprising:
(a) a first polypeptide comprising a first companion animal Fc region variant; and
(b) a second polypeptide comprising a second companion animal Fc region variant,
wherein the first companion animal Fc region variant and the second companion animal Fc region variant comprise complementary dimerization selectivity modules that promote dimerization between the first companion animal Fc region variant and the second companion animal Fc region variant, and
wherein the first polypeptide or the second polypeptide does not comprise an antibody.

32. The Fc construct of claim 31, further comprising a protein selected from the group consisting of EPO, CTLA4, LFA3, VEGFR1, VEGFR3, IL-1R, IL-4R, GLP-1 receptor agonist, and thrombopoietin binding peptide.

33. The Fc construct of claim 31 or 32, wherein the first companion animal Fc region variant and the second companion animal Fc region variant are canine Fc region variants.

34. The Fc construct of claim 33, wherein the first canine Fc region variant or the second canine Fc region variant comprises an amino acid sequence that is at least 95% identical to an amino acid sequence selected from the group consisting of SEQ ID NOs: 9 to 12.

35. The Fc construct of claim 34, wherein the first canine Fc region variant and the second canine Fc region variant each comprises a protuberance or a cavity, wherein if the first canine Fc region comprises a protuberance, the second canine Fc region comprises a cavity, and wherein if the first canine Fc region comprises a cavity, the second canine Fc region comprises a protuberance.

36. The Fc construct of claim 34 or 35, wherein the first canine Fc region variant and the second canine Fc region variant each comprises amino acid substitutions selected from the group consisting of:
(a) S354C and T366W in the first canine Fc region variant; and T366S, L368A, Y407V, and Y349C in the second canine Fc region variant;
(b) T366W in the first canine Fc region variant; and T366S, L368A, and Y407V in the second canine Fc region variant;
(c) R392D and K409D in the first canine Fc region variant; and E356K and D399K in the second canine Fc region variant;
(d) S364H and F405A in the first canine Fc region variant; and Y349T and T394F in the second canine Fc region variant;
(e) F405L in the first canine Fc region variant; and K409R in the second canine Fc region variant;
(f) T366L, R392L, and T394W in the first canine Fc region variant; and L351Y, F405A, and Y407V in the second canine Fc region variant;
(g) K360E and K409W in the first canine Fc region variant; and S347R, D399V, and F405T in the second canine Fc region variant;
(h) Y349C, K360E, and K409W in the first canine Fc region variant; and S347R, S354C, D399V, and F405T in the second canine Fc region variant;
(i) K370E and K409W in the first canine Fc region variant; and E357N, D399V, and F405T in the second canine Fc region variant;
(j) K360D, D399M, and Y407A in the first canine Fc region variant; and Q345R, S347R, T366V, and K409V in the second canine Fc region variant;
(k) Y349S, T366M, K370Y, and K409V in the first canine Fc region variant; and E356G, E357D, S364Q, and Y407A in the second canine Fc region variant;
(l) L351 D and L368E in the first canine Fc region variant; and L351K and T366K in the second canine Fc region variant;
(m) L368D and K370S in the first canine Fc region variant; and E356Q and S364K in the second canine Fc region variant; or
(n) T366Y in the first canine Fc region variant; and T366S, L368A, and Y407T in the second canine Fc region variant;
wherein the amino acid positions are based on EU numbering.

37. The Fc construct of any one of claims 33-36, wherein the first canine Fc region variant comprises a first charged region and the second canine Fc region variant comprises a second charged region, and wherein the first charged region forms a charge pair with the second charged region.

38. The Fc construct of claim 37, wherein the first charged region comprises a basic amino acid residue and the second charged region comprises an acidic amino acid residue.

39. The Fc construct of claim 38, wherein the first canine Fc region variant and the second canine Fc region variant further comprise CH1 domains comprising the following amino acid substitutions:
S183D in the first canine Fc region variant; and S183K in the second canine Fc region variant.

40. The Fc construct of claim 38 or 39, wherein the first canine Fc region variant and the second canine Fc region variant comprise CH3 domains comprising amino acid substitutions selected from the group consisting of:
(a) K409D in the first canine Fc region variant and D399K in the second canine Fc region variant;
(b) K390D and K409D in the first canine Fc region variant; and E356K and D399K in the second canine Fc region variant;
(c) K390D and K409D in the first canine Fc region variant; and E357K and D399K in the second canine Fc region variant; and
(d) K370D and K409D and in the first canine Fc region variant; and E357K and D399K in the second canine Fc region variant.

41. The Fc construct of any one of claims 38-40, wherein the first canine Fc region variant and the second canine Fc region variant further comprise CL domains comprising the following amino acid substitutions:
S176K in the first canine Fc region variant; and S176D in the second canine Fc region variant.

42. The Fc construct of any one of claims 33-41, wherein the first canine Fc region variant and the second canine Fc region variant further comprise at least one of the following amino acid substitutions:
(a) 252Y and, optionally, at least one amino acid substitution selected from the group consisting of 251 D or 251E; 285N or 285D; 286D; 307Q; 308P; 315D; 430A or 430K; 433K; 435Y; and 436H;
(b) 252M and, optionally, at least one amino acid substitution selected from the group consisting of 251 D or 251E; 256D or 256F; 285N or 285D; 286D; 307Q; 308P; 315D; 430A or 430K; 433K; 435Y; and 436H;
(c) 434R;
(d) 426Y and, optionally, at least one amino acid substitution selected from the group consisting of 286F, 286W, 286L, or 286Y; 312P; 434R; and 436H;
(e) 426H and, optionally, at least one amino acid substitution selected from the group consisting of 286F, 286W, 286L, or 286Y; 312P; 434R; and 436H;
(f) 426F and, optionally, at least one amino acid substitution selected from the group consisting of 286F, 286W, 286L, or 286Y; 312P; 434R; and 426H; and
(g) 434R and, optionally, at least one amino acid substitution selected from the group consisting of 286L; 286Y; 312P; and 436H;
wherein the amino acid positions are based on EU numbering.

43. The Fc construct of claim 31 or 32, wherein the first companion animal Fc region variant and the second companion animal Fc region variant are feline Fc region variants.

44. The Fc construct of claim 43, wherein the first feline Fc region variant or the second feline Fc region variant comprises an amino acid sequence that is at least 95% identical to an amino acid sequence selected from the group consisting of SEQ ID NOs: 19 to 21.

45. The Fc construct of claim 44, wherein the first feline Fc region variant and the second feline Fc region variant each comprises a protuberance or a cavity, wherein if the first feline Fc region comprises a protuberance, the second feline Fc region comprises a cavity, and wherein if the first feline Fc region comprises a cavity, the second feline Fc region comprises a protuberance.

46. The Fc construct of claim 44 or 45, wherein the first feline Fc region variant and the second feline Fc region variant comprise amino acid substitutions selected from the group consisting of:
(a) T366W in the first feline Fc region variant; and T366S, L368A, and Y407V in the second feline Fc region variant;
(b) T366W in the first feline Fc region variant; and T366S, L368A, and Y398T in the second feline Fc region variant;
(c) A354C and T366W in the first feline Fc region variant; and Y349C, T366S, L368A, and Y407V in the second feline Fc region variant;
(d) R392D and K409D in the first feline Fc region variant; and E356K and D399K in the second feline Fc region variant;
(e) S364H and F405A in the first feline Fc region variant; and Y349T and T394F in the second feline Fc region variant;
(f) F405L in the first feline Fc region variant; and K409R in the second feline Fc region variant;
(g) T366L, R392L, and T394W in the first feline Fc region variant; and L351Y, F405A, and Y407V in the second feline Fc region variant;
(h) R360E and K409W in the first feline Fc region variant; and Q347R, D399V, and F405T in the second feline Fc region variant;
(i) Y349C, R360E, and K409W in the first feline Fc region variant; and Q347R, A354C, D399V, F405T in the second feline Fc region variant;
(j) K370E and K409W in the first feline Fc region variant; and E357N, D399V, and F405T in the second feline Fc region variant;
(k) R360D, D399M, and Y407A in the first feline Fc region variant; and E345R, Q347R, T366V, and K409V in the second feline Fc region variant;
(l) Y349S, K370Y, T366M, and K409V in the first feline Fc region variant; and E356G, E357D, S364Q, and Y407A in the second feline Fc region variant;
(m) L351 D and L368E in the first feline Fc region variant; and L351K and T366K in the second feline Fc region variant;
(n) L368D and K370S in the first feline Fc region variant; and E356Q and S364K in the second feline Fc region variant; and
(o) T366Y in the first feline Fc region variant; and T366S, L368A, and Y407T in the second feline Fc region variant;
wherein the amino acid positions are based on EU numbering.

47. The Fc construct of any one of claims 43-46, wherein the first feline Fc region variant comprises a first charged region and the second feline Fc region variant comprises a second charged region, and wherein the first charged region forms a charge pair with the second charged region.

48. The Fc construct of claim 47, wherein the first charged region comprises a basic amino acid residue and the second charged region comprises an acidic amino acid residue.

49. The Fc construct of claim 48, wherein the first feline Fc region variant and the second feline Fc region variant further comprise CH1 domains comprising the following amino acid substitutions:
S183D in the first feline Fc region variant; and S183K in the second feline Fc region variant.

50. The Fc construct of claim 48 or 49, wherein the first feline Fc region variant and the second feline Fc region variant comprise CH3 domains comprising amino acid substitutions selected from the group consisting of:
(a) K409D in the first feline Fc region variant and D399K in the second feline Fc region variant; and
(b) K370D and K409D and in the first feline Fc region variant; and E357K and D399K in the second feline Fc region variant.

51. The Fc construct of any one of claims 48-50, wherein the first feline Fc region variant and the second feline Fc region variant further comprise CL domains comprising the following amino acid substitutions:
S176K in the first feline Fc region variant; and S176D in the second feline Fc region variant.

52. The Fc construct of any one of claims 43-51, wherein the first feline Fc region variant and the second feline Fc region variant further comprise at least one of the following amino acid substitutions:
(a) at least one amino acid substitution selected from the group consisting of 286E; 311V; and 428Y;
(d) two or more amino acid substitutions selected from the group consisting of 286E; 311V; and 428Y; and
(c) 286E, 311V, and 428Y;
wherein the amino acid positions are based on EU numbering.

53. A pharmaceutical composition comprising (i) the bispecific antibody of any one of claims 1-30 or the Fc construct of any one of claims 31-52, and (ii) a pharmaceutically acceptable excipient.

54. A nucleic acid or nucleic acids encoding the bispecific antibody of any one of claims 1-30 or the Fc construct of any one of claims 31-52.

55. An expression vector or expression vectors comprising the nucleic acid or nucleic acids of claim 54.

56. A host cell comprising the nucleic acid or nucleic acids of claim 54 or the expression vector or expression vectors of claim 55.

57. A method of making a bispecific antibody or an Fc construct, the method comprising:
(a) providing a nucleic acid or nucleic acids of claim 54;
(b) expressing the nucleic acid or nucleic acids in a host cell culture, thereby producing the bispecific antibody or the Fc construct; and, optionally,
(c) collecting the bispecific antibody or the Fc construct produced in (ii) from the host cell culture.

58. The method of claim 57, wherein the host cell culture comprises (i) one population of host cells expressing both the first companion animal Fc region variant and the second companion animal Fc region variant or (ii) two populations of host cells comprising a first population expressing the first companion animal Fc region variant and a second population expressing the second companion animal Fc region variant.

59. A method of treating or preventing a canine disease or disorder in a dog in need thereof, the method comprising administering an effective amount of a composition comprising the bispecific antibody of any one of claims 9-19, the Fc construct of any one of claims 33-42, or the pharmaceutical composition of claim 53 to the dog.

60. The method of claim 59, wherein the canine disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

61. The method of claim 59, wherein the canine disease or disorder is atopic dermatitis, allergic dermatitis, osteoarthritic pain, arthritis, anemia, or obesity.

62. The bispecific antibody of any one of claims 9-19, the Fc construct of any one of claims 33-42, or the pharmaceutical composition of claim 53 for use in treatment or prevention of a canine disease or disorder in a dog in need thereof.

63. The bispecific antibody or the Fc construct for use or the pharmaceutical composition for use of claim 62, wherein the canine disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

64. The bispecific antibody or the Fc construct for use or the pharmaceutical composition for use of claim 62, wherein the canine disease or disorder is atopic dermatitis, allergic dermatitis, osteoarthritic pain, arthritis, anemia, or obesity.

65. A method of treating or preventing a feline disease or disorder in a cat in need thereof, the method comprising administering an effective amount of a composition comprising the bispecific antibody of any one of claims 20-30, the Fc construct of any one of claims 43-52, or the pharmaceutical composition of claim 53 to the cat.

66. The method of claim 65, wherein the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

67. The method of claim 65, wherein the feline disease or disorder is atopic dermatitis, allergic dermatitis, osteoarthritic pain, arthritis, anemia, or obesity.

68. The bispecific antibody of any one of claims 20-30, the Fc construct of any one of claims 43-52, or the pharmaceutical composition of claim 53 for use in treatment or prevention of a feline disease or disorder in a cat in need thereof.

69. The bispecific antibody or the Fc construct for use or the pharmaceutical composition for use of claim 68, wherein the feline disease or disorder is an allergic disease, a chronic pain, an acute pain, an inflammatory disease, an autoimmune disease, an endocrine disease, a gastrointestinal disease, a cardiovascular disease, a renal disease, a fertility related disorder, an infectious disease, or a cancer.

70. The bispecific antibody or the Fc construct for use or the pharmaceutical composition for use of claim 68, wherein the feline disease or disorder is atopic dermatitis, allergic dermatitis, osteoarthritic pain, arthritis, anemia, or obesity.
